(19) United States
(12) Patent Application Publication
(10) Pub. No.: US 2024/0395397 A1
(43) Pub. Date: Nov. 28, 2024

(54) INSTRUCTIONAL POINT OF CARE DEVICE FOR SAMPLE HANDLING

(71) Applicant: CALIFORNIA INSTITUTE OF TECHNOLOGY, Pasadena, CA (US)

(72) Inventors: Rustem F ISMAGILOV, Altadena, CA (US); Minkyo LEE, Pasadena, CA (US); Si Hyung JIN, Pasadena, CA (US); Matthew M COOPER, Pasadena, CA (US); Roey LAZAROVITS, Pasadena, CA (US)

(21) Appl. No.: 18/667,828

(22) Filed: May 17, 2024

Related U.S. Application Data
(60) Provisional application No. 63/468,920, filed on May 25, 2023.

(51) Int. Cl. G16H 40/20

(52) U.S. Cl. CPC G16H 40/20

(57) ABSTRACT

An interactive modular device for instructing and assisting users on sample handling processes using features to guide a user through the processes in real time. The modules of the device include a processing module (e.g., pooling module), a sample-container module (e.g., tube module), a container-closure module (e.g., cap module), a sample-transfer module (e.g., pipette module), a scanning module (e.g., single barcode module), a multi-scanning module (e.g., multi-barcode module), a waste container module, and a display module. The features include mechanisms to determine presence and absence of items used for sample handling and visual, audible, and tactile indicators in real time.

### CROSS REFERENCE TO RELATED APPLICATIONS

The present application claims priority to U.S. Prov. App. No. 63/468,920 filed on May 25, 2023, the contents of which are incorporated by reference in their entirety.

### BACKGROUND

### 1. Field

The present disclosure relates to interactive devices, and more particularly, to methods and devices for helping users perform sample-handling procedures successfully while minimizing handling errors and risks.

### 2. Description of Related Art

The present disclosure describes a novel instructional point-of-care device for sample handling that is a device that teaches an untrained user how to perform a hands-on sample handling protocol. Among various sample handling procedures, the present disclosure describes the application of the instructional point-of-care device to sample pooling.

There is a growing demand for community-based screening (e.g., for communicable diseases, including respiratory diseases and STDs), creating the need to develop affordable, high-throughput diagnostic testing. Many communities—in the U.S. and globally—lack the resources and trained personnel to provide these needed healthcare services.

Individual molecular diagnostic tests are characterized as being too expensive for those in a community where expensive high-throughput automated medical devices do not exist due to limited budgets. Further, the automated devices needed to run these molecular tests are limited to Clinical Laboratory Improvement Amendments (CLIA) certified laboratories. FIG. 1A illustrates the high-throughput processing that may be performed by a skilled worker using an automated system such as the Roche Cobas 6800 system. In recent years, many medical device companies have developed more affordable, low-complexity CLIA-waived diagnostic devices. However, the CLIA-waived devices have low throughput; only a single test of an individual sample can be run at a time. FIG. 1B illustrates the low-throughput processing that may be performed by a relatively unskilled worker using a CLIA-waived system such as the Cepheid GeneXpert Xpress system. Low-throughput testing slows large-scale screening efforts and increases the total cost of testing. These problems can lead to delayed diagnoses and further spread of highly communicable diseases.

One strategy for improving throughput while reducing costs is sample pooling processing, wherein multiple individuals' samples (liquid or swabs) are collected into a single test tube. Pooling can increase testing throughput and reduce the total cost of testing to 25-50%. However, most hands-on diagnostic sample processing (including pooling protocols) is complicated and requires rigorous training under biosafety regulations and Center for Disease Control (CDC) or Food and Drug Administration (FDA) guidelines. Thus, pooled sample processing should be performed only by trained healthcare workers to avoid high risks of cross-contamination, biosafety issues, human errors, and mistakes. FIG. 1C illustrates sample pooling processing where skilled workers are used to process pooled samples and relatively unskilled workers perform individual sample testing using a CLIA-waived system such as the Cepheid GeneXpert Xpress system.

Sample pooling testing is a strategy to dramatically increase assay throughput while minimizing the logistics required for running multiple tests. Pooled testing is a time- and cost-saving approach commonly used to screen a large number of individuals for infectious diseases. The pooled test works by collecting a series of individual samples into one common pool. If the pooled test result is negative, all samples within the pool are diagnosed as negative. If a pooled test is positive, each sample in that pool will be tested individually to determine which samples are positive. From the pooling, fewer tests will be run overall, indicating that fewer testing supplies are used, and more tests can be run simultaneously. Therefore, the sample pooling testing strategy is most efficient in areas with low prevalence since most results are expected to be negative. FIG. 2 illustrates the use of sample pooling to reduce the number of tests and to perform community screening, where a negative result for a pooled test will indicate that no further tests for individuals in the pooled sample will be required.

Communities in which healthcare services are lacking also tend to be those in which there is a lack of higher education and professional training programs. Thus, few (or no) skilled people in these communities have the training to perform the necessary diagnostic workflows. This lack of trained healthcare workers compounds the poor distribution of healthcare, leading to inefficient centralized diagnostic testing rather than local, point-of-care testing.

There exists a need in the art for an instructional point-of-care (POC) sample pooling device that can simultaneously provide professional healthcare training to inexperienced people who need jobs and provide communities with a low-cost and high-throughput community screening resource that will improve healthcare at the point of care. Such a medical device can significantly impact both community healthcare services and the social economy. Sample pooling reduces costs and increases screening throughput, and the use of local, untrained workers makes this resource accessible to local communities. As a professional training tool, such a device will support training workers in healthcare diagnostics and create high-quality jobs accessible to inexperienced people.

### SUMMARY

Described herein are according to embodiments of the present invention that provide for methods, systems, devices and kits for interactive processing of samples.

One embodiment is an instructional medical device comprising a modular assembly, wherein the modules of the modular assembly comprise: one or more sample-transfer modules (e.g., pipette module), wherein each sample-transfer module is configured to hold at least one sample-transfer element tray (e.g., a pipette tray) and one or more sample-transfer elements (e.g., pipettes); a scanning module (e.g., single barcode module) configured to read one or more identifiers (e.g., barcodes) from an item; a processing module (e.g., pooling module) configured to hold one or more post-processing sample containers (e.g., a pooling tube), at least one post-processing sample container closure tray (e.g., a pooling cap tray), and one or more post-processing sample container closures (e.g., a pooling tube cap); one or more sample-container modules (e.g., tube module), wherein each sample-container module is configured to hold at least one sample container tray (e.g., a sample tube tray) and one or more sample containers (e.g., sample tubes); one or more container-closure modules (e.g., cap module), wherein each container-closure module is configured to hold at least one sample container closure tray (e.g., a cap tray) and one or more sample container closures (e.g., sample tube caps); one or more multi-scanning modules (e.g., multi-barcode module), wherein each multi-scanning module is configured to read barcodes from the sample container tray (e.g., sample tube tray) and the one or more sample containers; a waste container module (e.g., waste-bin module) configured to contain one or more used elements (e.g., used pipettes, used container trays) disposed of during the sample handling processes; and a display module configured to display information related to the instructional medical device, and wherein the instructional medical device further comprises an electronics system communicating to components within the modules of the modular assembly.

Another embodiment is a method for sample handling using a sample handling device having object sensing elements, the method comprising: detecting disposal of one or more materials being used, including cleaning towels or wipes for cleaning before the sample handling process, with at least one object sensing element, at least one motion sensing element, or at least one weight sensing element; providing status to the user regarding the disposal of material into a disposal container with at least one perceptible indication element of the sample handling device; detecting presence of a sample container tray holding one or more sample containers, a sample-transfer element tray (e.g., a pipette tray) holding one or more sample-transfer elements, a sample container closure tray (e.g., a cap tray) having one or more sample container closure detection wells, a post-processing sample container closure tray (e.g., a pooling cap tray), and one or more any materials being used during the process in the sample handling device with at least one object sensing element; providing status to a user regarding the presence of the sample container tray, sample container closure tray, sample-transfer element tray, and the post-processing sample container closure tray with at least one perceptible indication element of the sample handling device; detecting presence of a post-processing sample container in a post-processing sample container holder of the sample handling device and a post-processing sample container closure in the post-processing sample container closure tray in the sample handling device with at least one object sensing element; providing status to the user regarding the presence of the post-processing sample container and the post-processing sample container closure tray with a least one perceptible indication element of the sample handling device; detecting presence of a sample container closure in a sample container closure detection well with at least one object sensing element; providing status to the user regarding the presence of the sample container closure with at least one least one perceptible indication element of the sample handling device; detecting removal of a sample-transfer element from the sample-transfer element tray with at least one object sensing element; providing status to the user regarding the removal of the sample-transfer element with at least one perceptible indication element of the sample handling device; detecting disposal of one or more materials being used, including a plastic wrapping bag of sample-transfer elements, with at least one object sensing element, at least one motion sensing element, or at least one weight sensing element; providing status to the user regarding the disposal of material into a disposal container with at least one perceptible indication element of the sample handling device; detecting dispensation of material from a sample tube into the post-processing sample container using the sample-transfer element with at least one object sensing element or at least one weight sensing element; providing status to the user regarding the dispensation of material into the post-processing sample container with at least one perceptible indication element of the sample handling device; detecting disposal of one or more materials being used, including one or more used sample-transfer elements, with at least one object sensing element, at least one motion sensing element, or at least one weight sensing element; providing status to the user regarding the disposal of material into a disposal container with at least one perceptible indication element of the sample handling device; detecting removal of the post-processing sample container closure from the post-processing sample container closure tray with at least one object sensing element; providing status to the user regarding the removal of the post-processing sample container closure with at least one perceptible indication element of the sample handling device; detecting removal of the post-processing sample container from the post-processing sample container holder with at least one object sensing element; providing status to the user regarding the removal of the post-processing sample container with at least one perceptible indication element of the sample handling device; detecting removal of the sample container tray holding one or more sample containers from the sample handling device, with at least one object sensing element; providing status to the user regarding the removal of the sample container tray with at least one perceptible indication element of the sample handling device; detecting removal of the sample container closure tray from the sample handling device, with at least one object sensing element=; providing status to the user regarding the removal of the sample container closure with at least one perceptible indication element of the sample handling device; detecting disposal of one or more materials being used, including the used sample container closure, with at least one object sensing element, at least one motion sensing element, or at least one weight sensing element; providing status to the user regarding the disposal of material into a disposal container with at least one perceptible indication element of the sample handling device; detecting removal of the sample-transfer element tray from the sample handling device, with at least one object sensing element; providing status to the user regarding the removal of the sample-transfer element tray with at least one perceptible indication element of the sample handling device; detecting disposal of one or more materials being used, including—the used sample-transfer element tray, with at least one object sensing element, at least one motion sensing element, or at least one weight sensing element; providing status to the user regarding the disposal of material into a disposal container with at least one perceptible indication element of the sample handling device; detecting removal of the post-processing sample container closure tray from the sample handling device, with at least one object sensing element; providing status to the user regarding the removal of the post-processing sample container closure tray with at least one perceptible indication element of the sample handling device; detecting disposal of one or more materials being used, including the used post-processing sample container closure tray, with at least one object sensing element, at least one motion sensing element, or at least one weight sensing element; providing status to the user regarding the disposal of material into a disposal container with at least one perceptible indication element of the sample handling device; detecting disposal of one or more materials being used, including cleaning towels or wipes for cleaning after the sample handling process, with at least one object sensing element, at least one motion sensing element, or at least one weight sensing element; providing status to the user regarding the disposal of material into a disposal container with at least one perceptible indication element of the sample handling device; and detecting disposal of one or more materials being used, including used gloves, with at least one object sensing element, at least one motion sensing element, or at least one weight sensing element; providing status to the user regarding the disposal of material into a disposal container with at least one perceptible indication element of the sample handling device.

Another embodiment comprises a system for sample handling comprising: a main system coupled to a peripheral system, wherein the main system comprises: a main processor; a light emitting diode system coupled to the main processor, the light emitting diode system comprising a plurality of light emitting control units coupled to a plurality of light emitting diodes; a plurality of object detection sensors, motion detection sensors, and weighing scales coupled to the main processor; a digital storage interface unit coupled to the main processor; and a barcode scanner system coupled to the main processor, the barcode scanner system comprising at least one barcode scanner control unit and a plurality of barcode scanners, and wherein the peripheral system comprises: a peripheral processor; a display coupled to the peripheral processor; a storage element coupled to the peripheral processor, wherein the storage element stores graphics for use by the display; a sound generating element coupled to the peripheral processor; and, a timer coupled to the peripheral processor.

Another embodiment comprises an instructional system for supporting sample handling processes, helping a user learn how to do the processes, helping a user improve handling skills, or training a user on handling skill acquisition and adaptation to the processes without the instructional system, the system comprising a main system and a peripheral system, wherein the main system comprises: means for controlling illumination devices to guide the user on a current workflow status; means for operating barcode scanners to read barcodes from items handled during the sample handling processes; means for receiving signals from object detection sensors, motion detection sensors, and weighing scales to detect behaviors of the user; means for reading and writing data on external digital storage; and, means for communicating to the peripheral system, and wherein the peripheral system comprises; means for measuring time intervals for steps of the sample handling processes; means for generating alarm sounds; and, means for displaying graphics.

Another embodiment comprises a modular system for handling samples, wherein the system comprises one or more modules, and wherein the modular system comprises: one or more object detection sensors, wherein each object detection sensors comprises: an infrared emitter directed towards an object in a first direction and an infrared receiver directed towards an object in a second direction, wherein the first direction is offset from second direction and wherein the infrared receiver receives maximum infrared light when an object is present and minimum infrared light when the object is absent; one or more motion detection sensors, wherein each motion detection sensor comprises at least one passive infrared sensor that detects differential changes of infrared light due to a user's action in a detecting area; and one or more weighing scales, wherein each weighing scale detects the weight of an object, including a post-processing sample container or a material disposed of during a sample handling process.

Another embodiment comprises a kit for use in handling samples, the kit comprising one or more of the following compartments: a sample-transfer element compartment housing a sample-transfer module configured to hold one or more sample-transfer elements; a scanning compartment housing a scanning module configured to read one or more identifiers from an item; a processing compartment housing a processing module configured to hold one or more post-processing sample containers and one or more post-processing sample container closures; a sample-container compartment housing a sample-container module configured to hold one or more sample containers; a container-closure compartment housing a container-closure module configured to hold one or more sample container closures; a multi-scanning compartment housing a multi-scanning module configured to read one or more identifiers from the sample container tray and the one or more sample containers; and a display compartment housing a display module configured to display information, and wherein each compartment is configured to provide electrical communication to at least one other compartment.

Another embodiment comprises an instructional point-of-care device for helping a user learn how to do the processes, helping a user improve handling skills, or training a user on handling skill acquisition and adaptation to the processes without the instructional device, the device comprising: means for detecting presence and absence of sample containers, sample container closures, sample-transfer elements (e.g., a pipette), a post-processing sample container, or a post-processing sample container closure; means for detecting errors by the user in handling sample containers, sample container closures, sample-transfer elements (e.g., a pipette), a post-processing sample container, or a post-processing sample container closure; means for guiding the user in a process for correcting the detected errors; means for measuring a weight of a processed sample; means for guiding the user in a process for sample handling; and means for encouraging the user in transferring the fixed volume (or weight) of sample from each sample container.

### BRIEF DESCRIPTION OF THE SEVERAL VIEWS OF THE DRAWINGS

FIG. 1A illustrates high-throughput diagnostic processing using an automated system.

FIG. 1B illustrates low-throughput processing using a CLIA-waived system.

FIG. 1C illustrates an example of sample-pooling processing.

FIG. 2 illustrates the use of sample pooling to reduce the number of tests and to perform community screening.

FIG. 3 shows an instructional point-of-care device.

FIG. 4 shows the instructional point-of-care device of FIG. 3 with sample collection tubes, trays and pipettes in place.

FIG. 5 depicts an angle view of a tube module.

FIG. 6 shows a picture of the top of the tube module.

FIG. 7 shows a picture of one of the slots of the tube module and a tube detection sensor located within the slot.

FIG. 8 shows a picture of the internal structure of the tube module.

FIG. 9 shows a horizontal cross section of the tube module.

FIG. 10 shows a horizontal cross section of the tube module when a sample tube is not present.

FIG. 11 shows a vertical cross-section of the tube module when a tube tray is loaded.

FIG. 12 shows a vertical cross-section of the tube module when a tube tray is not loaded.

FIG. 13 shows a top view of a cap module and an adjoining multi-barcode module.

FIG. 14 shows a cap detection IR receiver within one cap detection well.

FIG. 15 shows a cap detection IR emitter within a cap detection well.

FIG. 16 shows a horizontal cross-section view of a cap module.

FIG. 17 shows a cap tray detection IR receiver in a detection well.

FIG. 18 shows a vertical cross-section view of the cap module.

FIG. 19 shows an internal view of six barcode scanners located within the multi-barcode module.

FIG. 20 shows sample tube barcodes on sample tubes positioned in the tube module and a pooling tube barcode on a tube positioned in a pooling module.

FIG. 21 shows a top cross-section view of the tube module and the multi-barcode module when sample collection tubes and tube tray are present inside tube module.

FIG. 22 shows an angle view of a pipette module with a pipette tray and transfer pipettes.

FIG. 23 shows a top view of the pipette module without the pipette tray installed.

FIG. 24 shows the internal structure of the pipette module without the pipette tray.

FIG. 25 shows the internal structure of the pipette module with the pipette tray installed with three pipettes.

FIG. 26 shows a cross-section of the pipette module with an individually wrapped pipette in the pipette tray.

FIG. 27 shows a cross-section of the pipette module without a pipette in the pipette tray.

FIG. 28 shows a vertical cross-section view of the pipette module when the pipette tray is loaded.

FIG. 29 shows a vertical cross-section view of the pipette module when the pipette tray is absent.

FIG. 30 shows a top view of a pooling module showing a pooling cap detection well and the pooling tube holder.

FIG. 31A shows a top view of the internal components of the pooling module.

FIG. 31B shows a close-up view of the internal components of the pooling module including a load cell and an electronic board.

FIG. 32A shows a view of the internal components of the pooling module under a small pooling tube module lid.

FIG. 32B shows an IR sensor used to detect a pooling cap held within a pooling cap tray.

FIG. 32C shows an IR sensor used to detect the pooling cap tray.

FIGS. 33A, 33B, and 33C show various views of the pooling cap tray.

FIG. 34 shows a side view of the small pooling tube module lid.

FIG. 35 shows a cross-section view of a part of the pooling module showing a 50 g load cell.

FIG. 36A shows an external view of a single-barcode module.

FIG. 36B shows an internal top view of the single-barcode module.

FIG. 37 shows a top view of the single-barcode module.

FIG. 38 illustrates the presentation of two types of 2D barcodes for selection of an operational mode.

FIG. 39A illustrates steps 1-9 of a training mode workflow.

FIG. 39B illustrates steps 10-18 of the training mode workflow.

FIG. 39C illustrates steps 19-27 of the training mode workflow.

FIG. 39D illustrates steps 28-34 of the training mode workflow.

FIG. 40A illustrates steps 1-9 of an expert mode workflow.

FIG. 40B illustrates steps 10-17 of the expert mode workflow.

FIG. 41 illustrates the use of symbolic and numerical markers.

FIG. 42 shows a process for locating a sample tray corresponding to a pooling tube with a reported positive test.

FIG. 43 shows a certification process.

FIG. 44 illustrates utilization of an instructional point-of-care device for learning through entertainment content.

FIG. 45 shows a system block diagram for the architecture of an instructional point-of-care device.

FIG. 46 shows a flow chart for an operational algorithm of a sample pooling workflow in a training mode.

FIG. 47 shows a flow chart for a Putting_on_gloves( ) function.

FIG. 48 shows a flow chart for a Disinfection( ) function.

FIG. 49 shows a first portion of a flow chart for a Tube_tray_detection( ) function.

FIG. 50 shows a second portion of the flow chart for the Tube_tray_detection( ) function.

FIG. 51 shows a flow chart for a Cap_tray_detection( ) function.

FIG. 52 show a first portion of a flow chart for a Pipette_tray_detection( ) function.

FIG. 53 shows a second portion of the flow chart for the Pipette_tray_detection( ) function.

FIG. 54 shows a flow chart for a Pooling_cap_tray_detection( ) function.

FIG. 55 shows a flow chart for a Pooling_tube_barcode_scan( ) function.

FIG. 56 shows the flow chart for the Pooling_tube_open( ) function.

FIG. 57 shows a flow chart for a Pooling_tube_detection( ) function.

FIG. 58 shows a flow chart for a Pooling( ) function.

FIG. 59 shows a first portion of a flow chart for an Opening_sample_tube task.

FIG. 60 shows a second portion of the flow chart for the Opening_sample_tube task.

FIG. 61 shows a third portion of the flow chart for the Opening_sample_tube task.

FIG. 62 shows a flow chart for a Picking_up_pipette function.

FIG. 63 shows a first portion of a flow chart for the Collecting_sample function.

FIG. 64 shows a second portion of the flow chart for the Collecting_sample function.

FIG. 65 shows a flow chart for a Closing_sample_tube function.

FIG. 66 shows a flow chart for a Pooling_tube_close( ) function.

FIG. 67 shows a flow chart for a Pooling_tube_removal( ) function.

FIG. 68 shows a flow chart for a Tube_tray_removal( ) function.

FIG. 69 shows a flow chart for a Cap_tray_removal( ) function.

FIG. 70 shows a flow chart for a Pipette_tray_removal( ) function.

FIG. 71 shows a flow chart for a Pooling_cap_tray_removal( ) function.

FIG. 72 shows a flow chart for a Taking_off_gloves( ) function.

FIG. 73 shows real-time detection functional blocks for 44 different behaviors.

FIG. 74 shows an example of Step 8 of a pooling workflow when the pool size is 3.

FIG. 75 shows an example of Step 15 of a pooling workflow during a pooling cycle for a first tube when the pool size is 3.

FIG. 76 shows an example of Step 27 of a pooling workflow when the pool size is 3.

FIG. 77 shows a block diagram of event handling tasks.

FIG. 78 shows a state machine for the control of each event handling task.

FIG. 79 shows the design of an instructional point-of-care device with a left-to-right workflow.

FIG. 80 shows the design of an instructional point-of-care device where a tube module and a pooling module are close to each other.

FIG. 81 shows the design of an instructional point-of-care device where a pipette module and a pooling module are far from each other.

FIG. 82 shows the design of an instructional point-of-care device where a tube module and a cap module are close to each other.

FIG. 83 shows the design of an instructional point-of-care device where a pooling cap detection well is located outside a pathway of pooling.

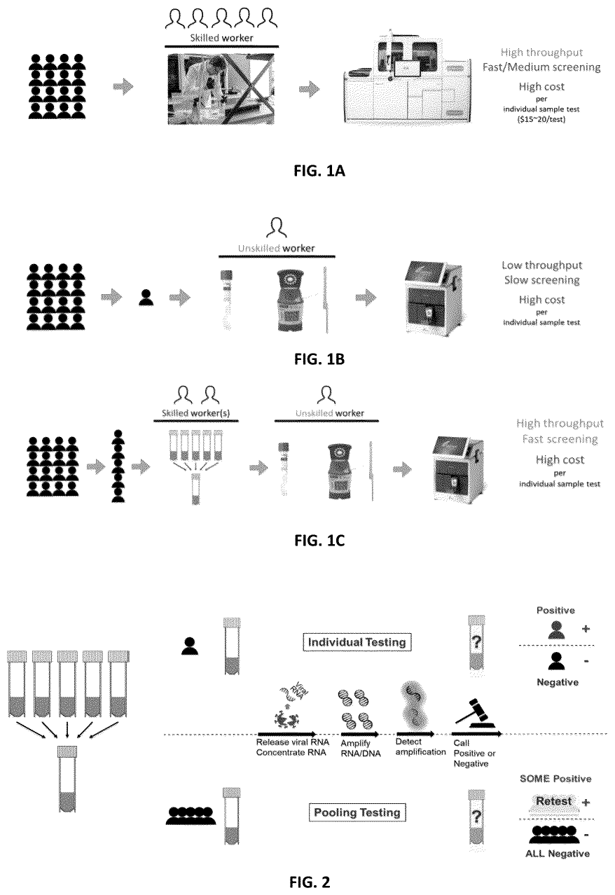

### DETAILED DESCRIPTION

The system and method of the present disclosure comprise a novel instructional point-of-care device for sample pooling, where the device teaches an untrained user how to perform a hands-on sample pooling protocol. The device detects the presence and absence of the sample tubes, sample tubes' caps, the sample-transfer elements (e.g., transfer pipettes), and other elements (e.g., wastes generated during the process) and catches human errors during the process. The device is interactive; it uses language-free graphics, colored lights, sounds, and other features to guide the user through the protocol step by step the correct way. Furthermore, the device measures the weight of every pooled liquid sample and calculates its volume to ensure balanced and accurate ratios among individual samples during pooling. The device helps workers, including untrained users and trained users, perform the complicated pooling procedures successfully with minimal risk of contamination and human error. The device also helps a worker, including untrained users and trained users, improve worker's handling skills, which include skills for accurate and reliable volume-transfer of samples. Lastly, the device provides a worker with training that can help the worker acquire good handling skills through the interaction with the device and perform the process accurately and reliably with minimal human errors by leveraging the gained handling skills independently without assistance from the device.

Embodiments of the methods, systems, devices and kits are described in additional detail below. The descriptions below present descriptions of elements of the embodiments using examples for some of the elements. For example, Aa pipette module comprises an example of a sample-transfer module element. A pipette tray comprises an example of a sample-transfer element tray element. A pipette comprises an example of a sample-transfer element. A single barcode module comprises an example of a scanning module element. An example of an identifier element comprises a barcode. A pooling module comprises an example of a processing module element. A pooling tube comprises an example of a post-processing sample container element. A pooling cap tray comprises an example of a post-processing sample container closure tray element. A tube module comprises an example of a sample-container module element. A sample tube tray comprises an example of a sample container tray element. A cap tray comprises an example of a sample container closure tray element. A sample tube cap comprises an example of a sample container closure element. A multi-barcode module comprises an example of a multi-scanning module element. A waste-bin module comprises an example of a waste container module element. Those skilled in the art understand that other modules, devices, or elements other than the examples described below may be within the spirit and scope of the present disclosure.

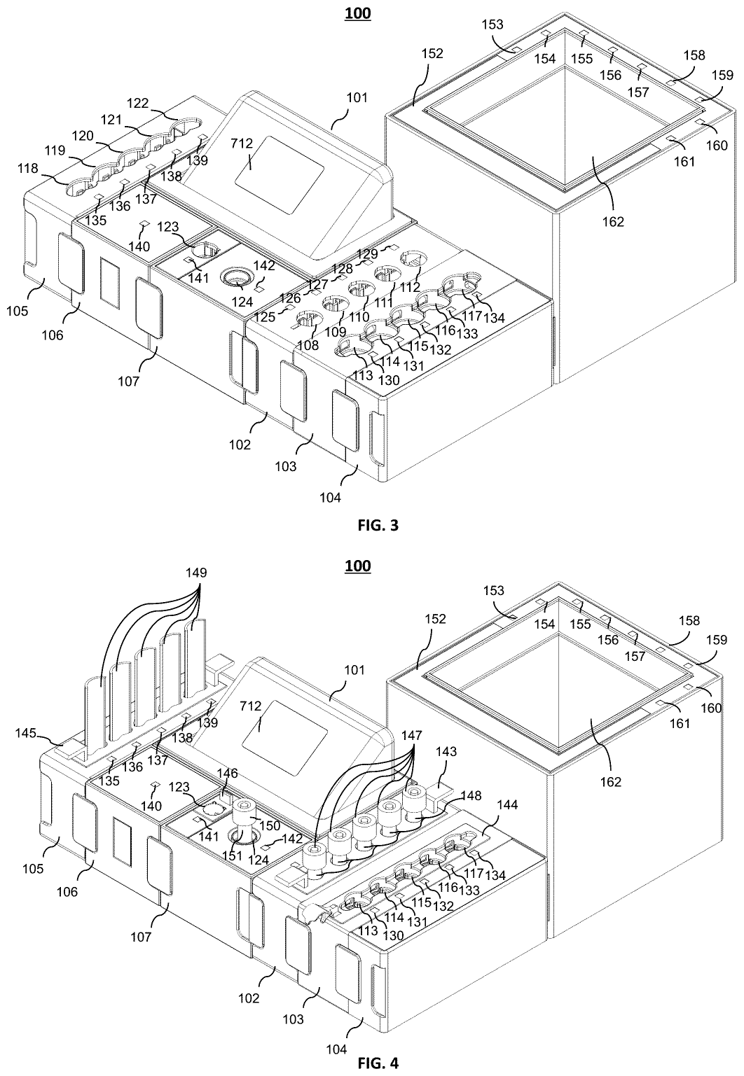

FIG. 3 shows an instructional point-of-care device 100 without sample collection tubes, tray and pipettes being shown. The device 100 has a modular configuration comprising the following modules: a pipette module 105, a single barcode module 106, a pooling module 107, a tube module 102, a cap module 103, a multi-barcode module 104, a waste-bin module 152, and a display module 101. The pipette module 105 has five pipette receiving slots 118, 119, 120, 121, 122 and five pipette receiving slot status light emitting diodes (LED) 135, 136, 137, 138, 139, where each LED corresponds to single pipette receiving slot 118, 119, 120, 121, 122. The single barcode module 106 has a barcode status LED 140. The pooling module 107 has pooling cap detection well 123, a pooling cap detection status LED 141, a pooling tube holder 124 and a pooling tube status LED 142. The tube module 102 has five sample collection tube receiving slots 108, 109, 110, 110, 111, 112 and five corresponding receiving slot status LEDs 125, 126, 127, 128, 129. The cap module 103 has five sample cap detection wells 113, 114, 115, 116, 117 and five corresponding cap detection status LEDs 130, 131, 132, 133, 134. The waste-bin module 152 has a disposal container 162 inside the waste-bin module 152 and nine disposal action status LED 153, 154, 155, 156, 157, 158, 159, 160, 161. The display module has a thin-film-transistor liquid-crystal display (TFT LCD) 712.

FIG. 4 shows the device 100 depicted in FIG. 3 with sample collection tubes, trays and pipettes in place. As shown in FIG. 4, five pipettes 149 held within a pipette tray 145 are positioned within the pipette module 105. A pooling cap tray 146 is positioned within the pooling cap detection well 123 of the pooling module. A pooling tube 151 with a pooling tube cap 150 is positioned within the pooling tube holder 124 of the pooling module. Five sample tubes 148 held within a sample tube tray 143 and having sample tube caps 147 are positioned within the tube receiving slots of the tube module 102. A cap tray 144 is positioned within the detection wells 113, 114, 115, 116, 117 of the cap module 103.

The instructional point of care device 100 provides for both instructional use of the device 100 to train unskilled workers on the proper procedures for pooling samples and use by more skilled workers to ensure that samples are quickly and correctly pooled for testing. For an unskilled worker, the device 100 provides a training mode to teach the unskilled worker how to perform the sample handling process properly without mistakes. For the skilled worker, the device 100 helps the trained user to perform the sample handling process without mistakes and accelerate the healthcare flow in diagnostics. As such, the individual modules 101, 102, 103, 104, 105, 106, 107 contain detection circuitry and display mechanisms to guide the user through the sample pooling process.

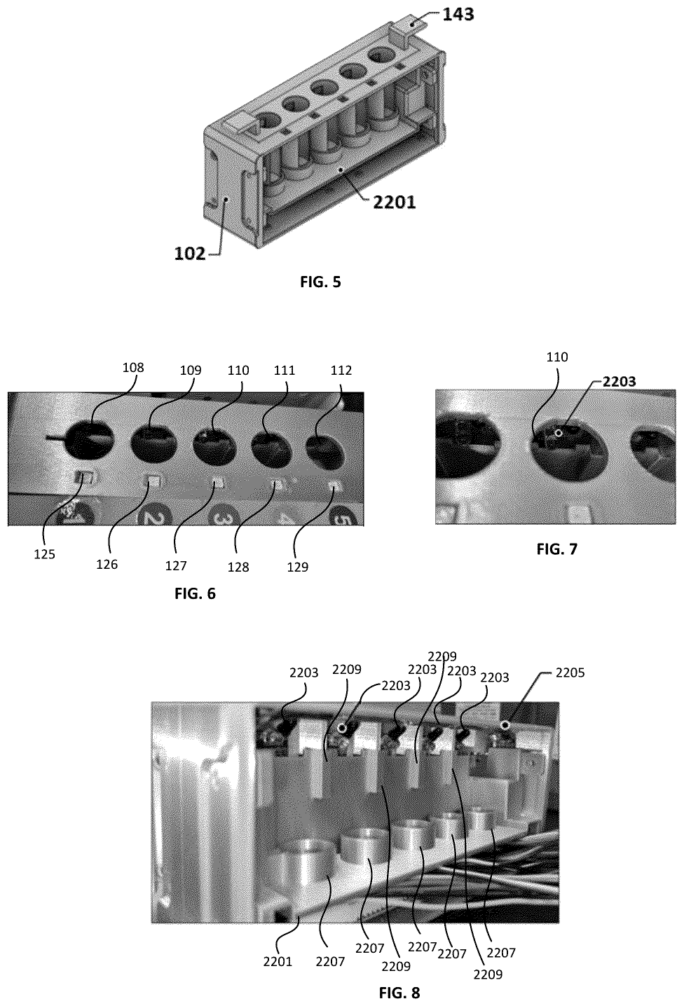

The tube mode 102 is configured to receive and hold sample tubes 148 from individuals to be pooled for testing. FIG. 5 depicts an angle view of the tube module 102 with the tube tray 143 installed and an inner plastic cover 2201. The tube module also contains five tube detection infrared (IR) sensors 2203 and one tube tray detection IR sensor 2205 that are not shown in FIG. 5. Each emitter of the IR sensors faces downward and each receiver of the IR sensors faces upward. These sensors are configured to only detect close objects, typically within 10 mm. The receiver IR sensor faces the opposite direction to prevent the receiver from detecting unwanted reflected light from surrounding objects.

FIG. 6 shows a picture of the top of the tube module 102 showing the five sample collection tube receiving slots 108, 109, 110, 110, 111, 112 and five corresponding receiving slot status LEDs 125, 126, 127, 128, 129. FIG. 7 shows a picture of one of the slots 110 and the tube detection sensor 2203 located within the slot 110. FIG. 8 shows a picture of the internal structure of the tube module 102. The five tube detection IR sensors 2203 corresponding to each tube receiving slot are shown along with the tray detection IR sensor 2205 located near the back of the module 102. The inner plastic cover 2201 is also shown. This plastic cover 2201 provides a circular structure 2207 at each slot location to receive a bottom of the sample tube and vertical structures 2209 to aid in guiding the sample tube into the circular structures 2207.

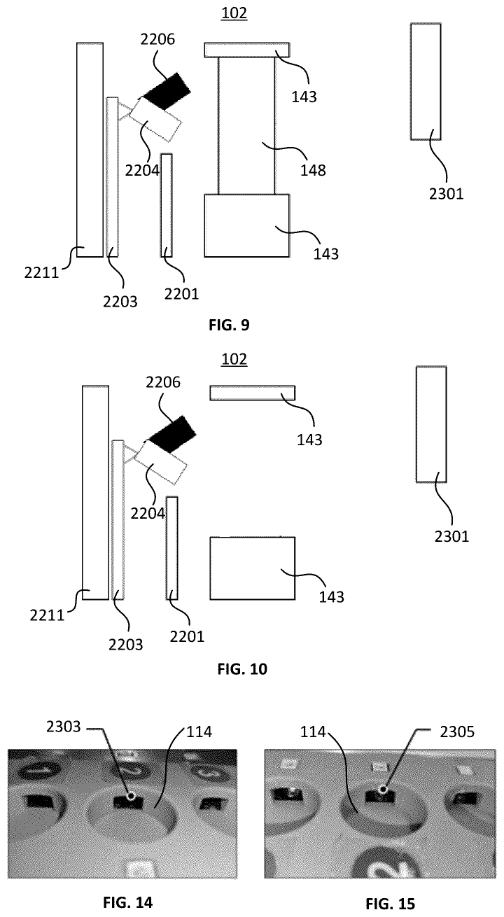

FIG. 9 shows a horizontal cross section of the tube module 102 with the IR sensors 2203 configured to detect sample collection tubes 148 in the tube tray 143 when positioned in the tube module 102. FIG. 9 also shows the outer wall 2301 of the adjoining cap module 103. An IR sensor 2203 with an emitter 2204 and a receiver 2206 covered by the inner plastic cover 2201 is fixed to the back wall 2211 of the outer tube module box 102. The IR emitter diode 2204 faces downwards and receiver 2206 faces upwards. When a sample collection tube 148 is present in a slot of the tube tray 143, the infrared light emitted from the emitter 2204 will be reflected by the sample tube 148, and some of the reflected light will travel to the IR receiver 2206 and be detected.

FIG. 10 shows a horizontal cross section of the tube module 102 when a sample tube 148 is not present. When the sample collection tube 148 is absent from the tube tray 143, the infrared light emitted from the emitter 2204 will be reflected by the tube tray 143 or the adjoining wall 2301 of the cap module 103. However, the reflected light will be detected with very weak intensity by the IR receiver facing upwards 2206.

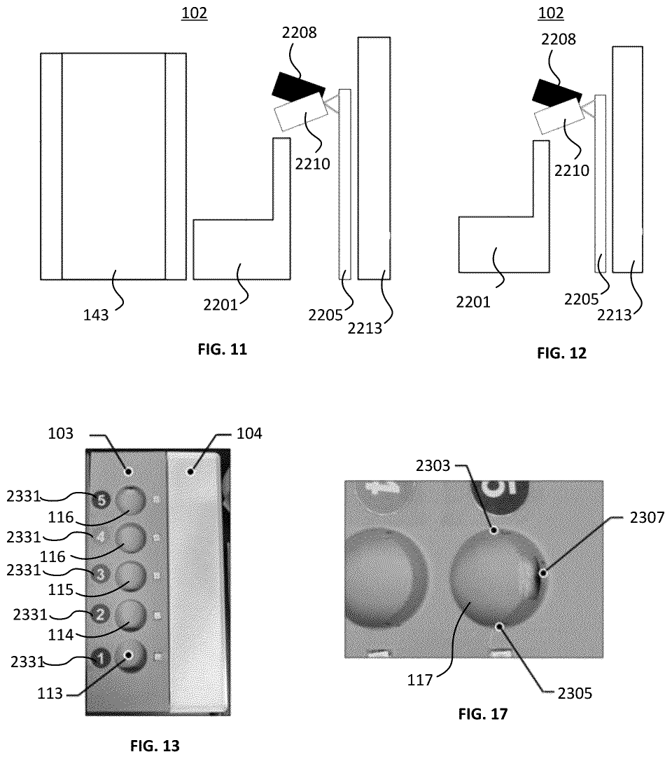

FIG. 11 shows a vertical cross-section of the tube module 102 when a tube tray 143 is loaded. The tray detection IR sensor 2205 covered by the inner plastic cover 2201 is fixed to a sidewall 2213 of the tube module 102. When tube tray 143 is present inside tube module 102, the infrared light emitted from the emitter 2208 will be reflected by tube tray 143, and some of the reflected light will travel to the IR receiver 2210 and be detected.

FIG. 12 shows a vertical cross-section of the tube module 102 when a tube tray 143 is not loaded. When the tube tray 143 is missing, the infrared light emitted from the emitter 2210 will be reflected by the inner plastic cover 2201, the adjoining wall of the cap module 102 (not shown), or other structures. However, the reflected light will be detected with very weak intensity by the IR receiver facing upwards 2208.

The cap mode 103 is configured to receive and hold the caps 147 of the sample tubes 148 from individuals to be pooled for testing. FIG. 13 shows a top view of the cap module 103 and the adjoining multi-barcode module 104. As shown in FIG. 13, the cap module has five cap detection wells 113, 114, 115, 116, 117 and the five corresponding cap detection status LEDs 130, 131, 132, 133, 134. FIG. 14 also shows that the cap detection wells are prominently labeled with numbers 2331 to aid the user in identifying the caps 147 placed in the wells.

Similar to the tube module 102, the cap module uses IR sensors to detect the presence of caps 147 within the cap detection wells 113, 114, 115, 116, 117. FIG. 14 shows a cap detection IR receiver 2303 within one cap detection well 114. FIG. 15 shows a cap detection IR emitter 2305 within the same cap detection well 114. As shown in FIG. 14 and FIG. 15, emitters 2305 and receivers 2303 of IR sensors (for tube caps 1147) face each other to detect only tube caps 147 inside the detection wells 113, 114, 115, 116, 117.

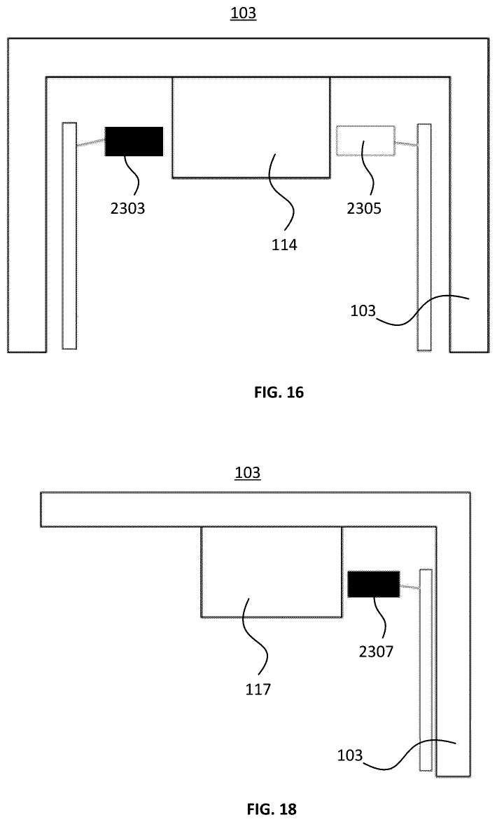

FIG. 16 shows a horizontal cross-section view of the cap module 103. As shown in FIG. 16, one IR emitter 2305 and receiver 2303 are fixed to the left wall and right wall of the cap module box 103, respectively. When a tube cap is absent from the detection well 114, IR light emitted from the emitter 2305 will travel to the receiver 2303 and be detected. When a tube cap is present in the well 114, the IR receiver 2303 will not be able to detect IR light emitted from the emitter 2305.

An IR receiver is used to detect the presence of the cap tray 144 at the cap detection wells 113, 114, 115, 116, 117. FIG. 17 shows the cap tray detection IR receiver 2307 in the detection well 117. As shown in FIG. 17, the cap detection well 117 also has a cap detection IR receiver 2303 and cap detection IR emitter 2305. FIG. 18 shows a vertical cross-section view of the cap module 103. One cap tray detection IR receiver 2307 is fixed to the sidewall of the cap module box 103. When the cap tray 144 is absent from the detection well 117, IR light emitted from the emitter 2305 will travel to the receiver 2307 and be detected. When the cap tray 144 is present in the well 117, IR light emitted from the emitter 2305 will be blocked by the wall of the cap tray 144, and the IR receiver 2307 will not be able to detect the emitted IR light.

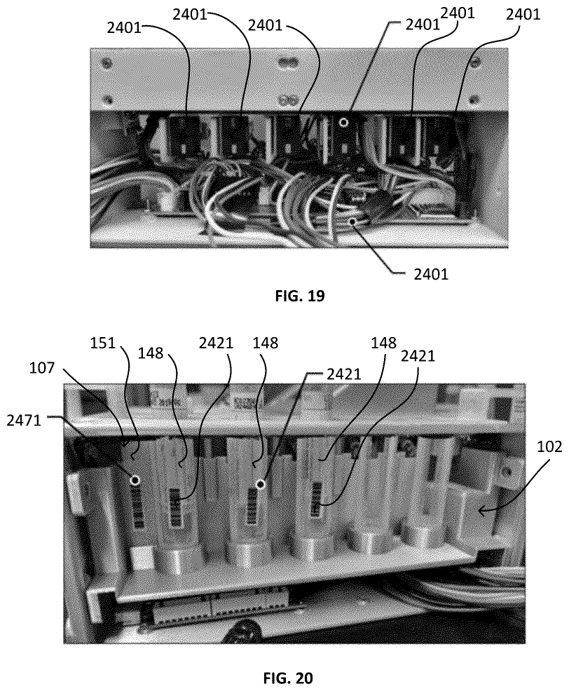

FIG. 19 shows an internal view of the six barcode scanners 2401 located within the multi-barcode module 104. The barcode scanners 2401 are used to scan the barcodes of tubes positioned within the pooling module 107 and the tube module 102. FIG. 19 also shows the electronic boards used to interface to the barcode scanners 2401. FIG. 20 shows sample tube barcodes 2421 on sample tubes 148 positioned in the tube module 102 and a pooling tube barcode 2471 on a tube 151 positioned in the pooling module 107.

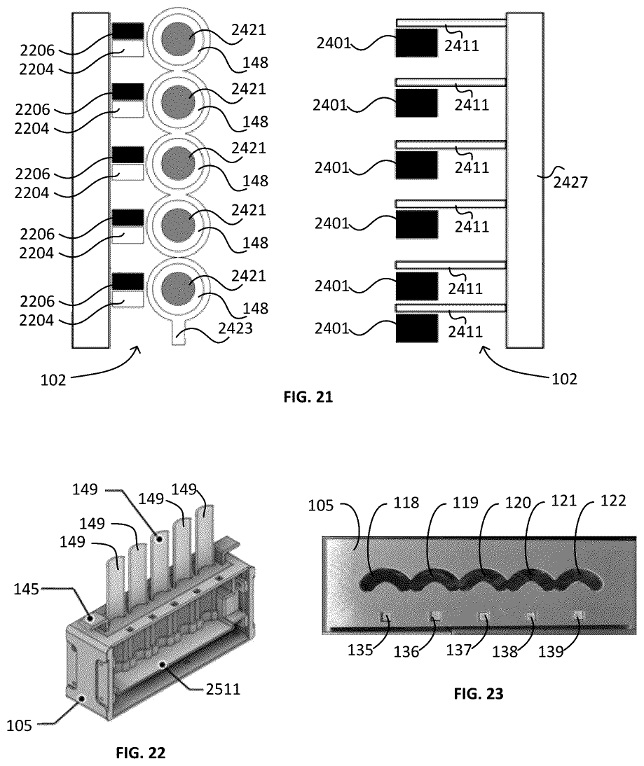

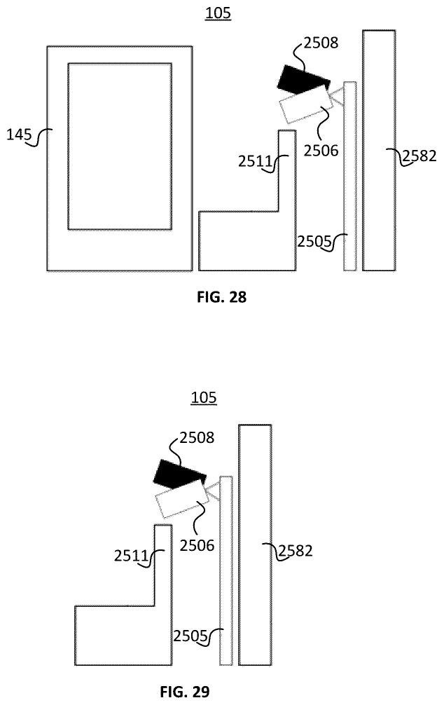

FIG. 21 shows a top cross-section view of the tube module 102 and the multi-barcode module 104 when sample collection tubes 148 and tube tray 143 are present inside tube module 102. The six barcode scanners 2401 are fixed to small plastic structures 2411 extruded from multi-barcode module chassis 2427. The scanners 2401 scan the barcodes when a microcontroller (not shown in FIG. 21) sends a command signal to each scanner 2401; otherwise, the scanners 2401 are in an idle state. The scanners 2401 can scan 1D and 2D codes, and each barcode contains unique information not to be overwritten by other barcodes. The distance between the scanners 2401 and sample tubes 148 is −60 mm. Thus, when the sample collection tubes 148 with the tube tray 143 are present inside the tube module 142, the barcode scanners 2401 can scan the tube tray barcode 2423 and the barcodes of sample tubes 2421. FIG. 28 also shows the sample tube emitters 2204 and receivers 2206 located within the tube module 102.

The pipette module 105 is configured to receive and hold individual pipettes 149 held within the pipette tray 145. FIG. 22 shows an angle view of the pipette module 105 with the pipette tray 145 and transfer pipettes 149. As shown in FIG. 22, the pipette module 107 has an inner pipette module plastic cover 2511 that receives the bottom portions of the transfer pipettes 149 when held in the pipette tray 145. FIG. 23 shows a top view of the pipette module 105 without the pipette tray 145 installed. FIG. 23 shows the individual pipette slots 118, 119, 120, 121, 122 and corresponding pipette slot status LEDs 135, 136, 137, 138, 139.

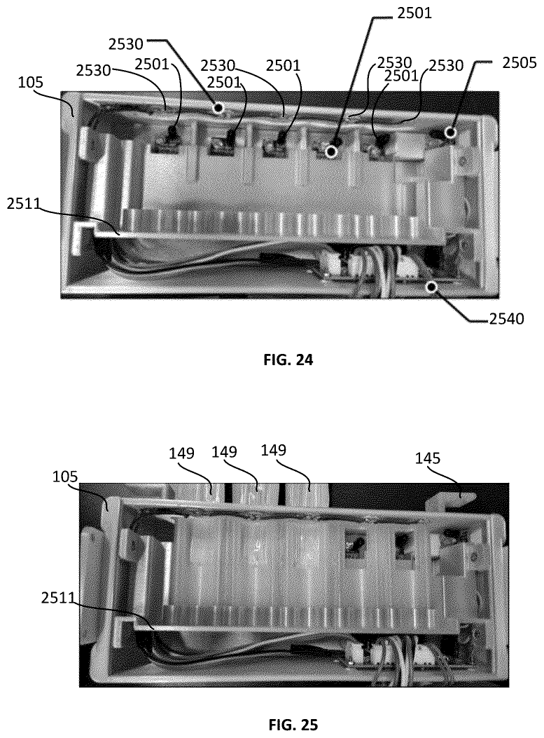

FIG. 24 shows the internal structure of the pipette module 105 without the pipette tray 145. The pipette module has five pipette-sensing IR sensors 2501 for detecting the presence of individual pipettes 149 and a pipette tray IR sensor 2505 for detecting the presence of the pipette tray 146. FIG. 24 also shows the LED sockets 2530 for the pipette slot status LEDs 135, 136, 137, 138, 139 and the pipette module electronic boards 2540 that interface to the sensors 2501, 2505 and LED sockets 2530. FIG. 25 shows the internal structure of the pipette module 105 with the pipette tray 145 installed with three pipettes 149.

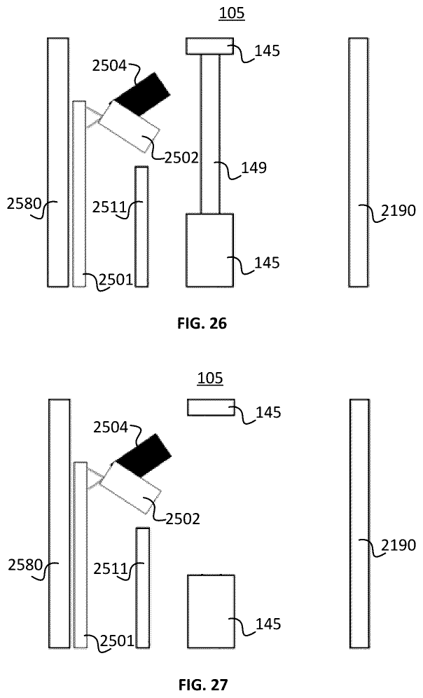

Similar to the tube module 102, each pipette-sensing IR sensor 2501 of the pipette module 105 has an emitter 2502 that faces downward and a receiver 2504 that faces upwards. These sensors 2501 only detect close objects within 10 mm. The IR sensor receiver 2504 faces the opposite direction to prevent the receiver 2504 from detecting unwanted reflected light from surrounding objects. FIG. 26 shows a cross-section of the pipette module 105 with an individually wrapped pipette 149 in the pipette tray 145. As shown in FIG. 26, the IR sensor 2501 has an emitter 2502 and a receiver 2504 covered by the inner pipette module plastic cover 2511. The IR sensors 2511 are fixed to the back wall 2580 of the pipette module 105. The IR emitter diode 2502 and receiver 2504 face downward and upwards, respectively. When an individually wrapped pipette 149 is present in a slot of the Pipette tray 145, the infrared light emitted from the emitter 2502 will be reflected by the sealed pipette 149, and some of the reflected light will travel to the IR receiver 2504.

FIG. 27 shows a cross-section of the pipette module 105 without a pipette 149 in the pipette tray 145. When an individually wrapped pipette 149 is absent from the pipette tray 145, the infrared light emitted from the emitter 2502 will be reflected by the pipette tray 145 or the walls 2190 of the display module 101 or single-barcode module 106. However, reflected light will be detected with very weak intensity by the IR receiver facing upwards 2504.

FIG. 28 shows a vertical cross-section view of the pipette module 105 when the pipette tray 145 is loaded. The pipette tray IR sensor 2505, covered by the pipette module plastic cover 2511, is fixed to a sidewall 2582 of the pipette module 105. When the pipette tray 145 is present inside the pipette module 105, the infrared light emitted from the emitter 2506 will be reflected by the pipette tray 145, and some of the reflected light will travel to the receiver 2508.

FIG. 29 shows a vertical cross-section view of the pipette module 105 when the pipette tray 145 is absent. When the pipette tray 145 is missing, the infrared light emitted from the emitter 145 will be reflected by the inner plastic cover 1511, the walls of the display module 101, or the single-barcode module 106, or other structures. However, reflected light will be detected with very weak intensity by the IR receiver 2508 facing upwards.

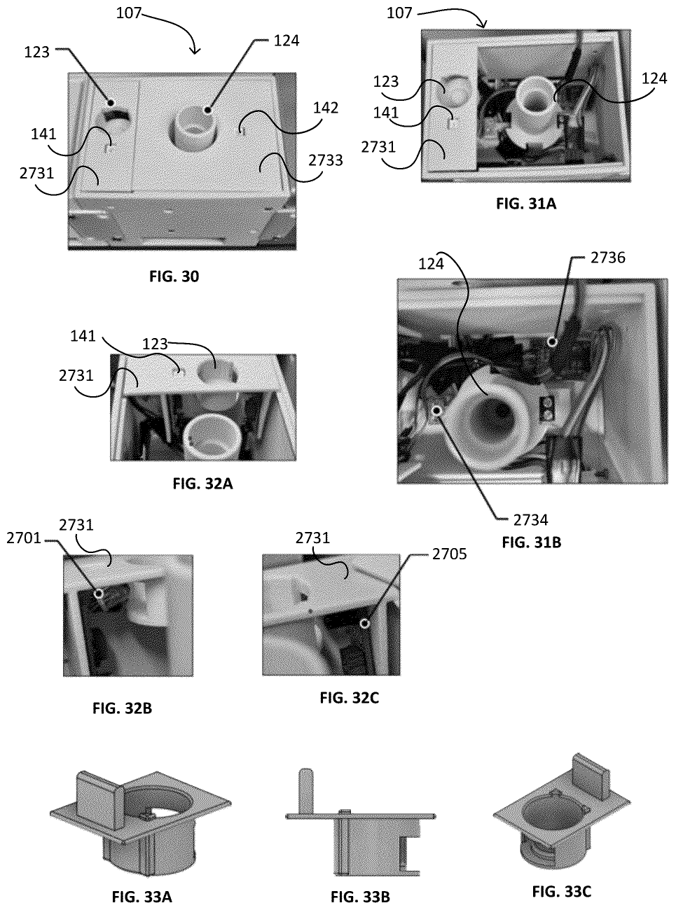

The pooling module 107 is configured to hold the pooling tube 151 containing the pooled samples and the pooling cap tray 146 holding the cap 150 for the pooling tube 151. FIG. 30 shows a top view of the pooling module 107 showing the pooling cap detection well 123 and the pooling tube holder 124. FIG. 30 also shows pooling cap detection status LED 141 positioned within a small pooling tube module lid 2731 and the pooling tube status LED 142 positioned within a large pooling tube module lid 2733.

FIG. 31A shows a top view of the internal components of the pooling module 107. FIG. 31B shows a close up view of the internal components including a load cell 2734 and an electronic board 2736 that interfaces to the load cell 2736, LEDs 141, 142 and sensors (described in additional detail below) of the pooling module 107. FIG. 32A shows a view of the internal components of the pooling module 107 under the small pooling tube module lid 2731. FIG. 32B shows the IR sensor 2701 used to detect the pooling cap 150 held within the pooling cap tray 146. FIG. 32C shows the IR sensor 2705 used to detect the pooling cap tray 146. FIGS. 33A, 33B, and 33C show various views of the pooling cap tray 146.

The pooling cap detector IR sensor 2701 has an emitter 2702 and receiver 2704 that face downwards and upwards, respectively, to detect only a close object. If the IR emitter 2702 and receiver 2704 of the IR sensor 2701 faced the same direction, the infrared light would be reflected by other surrounded objects easily, and the receiver 2704 would detect unwanted reflected light. Hence, having the emitter 2702 and receiver 2704 face different directions reduces the detection of unwanted light. The pooling cap tray detector IR sensor 2705 has only an IR receiver 2706. Since the IR receiver 2706 for the pooling cap tray 144 faces the IR emitter 2702 of the IR sensor for the pooling cap 150, the IR receiver 2706 for the pooling cap tray 144 can receive IR light when the pooling cap tray 144 is absent, and cannot receive the light when the tray 144 is present.

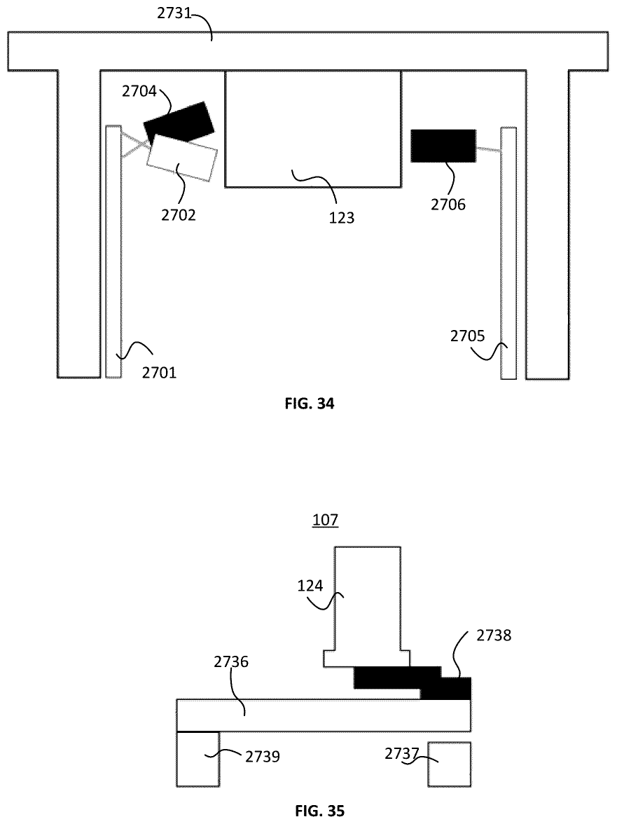

FIG. 34 shows a side view of the small pooling tube module lid 2731 with the pooling cap detection well 123, the pooling cap detection IR sensor 2701, and the pooling cap trap detection IR sensor 2705. The pooling cap detection IR sensor 2701 with emitter 2702 and receiver 2704 is fixed to the left leg of the plastic lid 2731, and the pooling cap tray detector IR sensor 2705 with one IR receiver 2706 is fixed to the right leg of the lid 2731. When the pooling cap 150 and pooling cap tray 143 are absent from the detection well 123, IR light emitted from the emitter 2702 on the left leg will travel to the receiver 2706 on the right leg and be detected. When the pooling cap tray 143 is loaded into the well 123, the IR receiver 2706 on the right leg will not be able to detect IR light emitted from the emitter 2702 on the left leg. In addition, the receiver 2704 on the left leg may receive very weak IR light reflected by surrounding objects due to the absence of pooling cap 150. When the pooling cap 150 is loaded into the well 123, IR light emitted from the emitter 2702 on the left leg will be reflected by the pooling cap 150, and the IR light will be detected by the IR receiver 2704 on the left leg.

A load cell is used to measure the weight of the liquid sample in the pooling tube 151 while the sample is being dispensed into the pooling tube 151. FIG. 35 shows a cross-section view of a part of the pooling module 107 showing a 50 g load cell 2736. The 50 g load cell 2736 with strain sensors is fixed to a plastic supporting structure 2739. A flexible plastic structure 2738 with the pooling tube holder 124 is attached to one end of the load cell 2736. Thus, when the pooling tube 151 is loaded in the holder 124, the flexible plastic structure 2738 and the load cell 2736 will be strained. The strain sensors will detect the movement. When the tube weight is tared, the device can measure the weight of the pooled liquid sample while the sample is collected into a pooling tube 151. A plastic supporting structure 2737 will prevent the over-strain of the load cell 2736 when a large amount of force is applied to the cell 2736.

The single-barcode module 106 is configured to read barcodes on pooling tubes 151, pooling tube caps 150 and other items held external to the module 106. FIG. 36A shows an external view of the single-barcode module 106 with the barcode module status LED 140 and barcode module scanner window 2610. FIG. 36B shows an internal top view of the single-barcode module 106 that shows the single-barcode module barcode scanner 2620 and the electronic board 2630 that interfaces to the barcode scanner 2620.

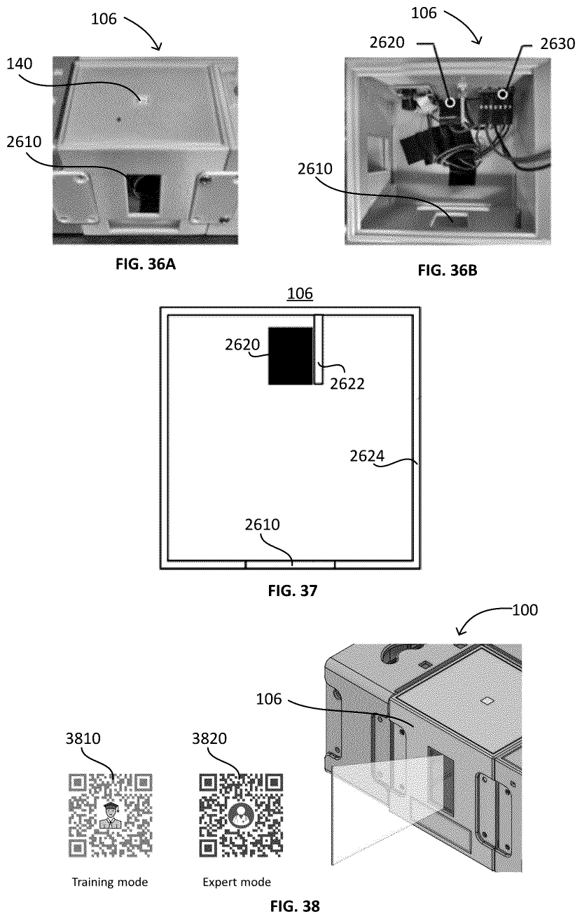

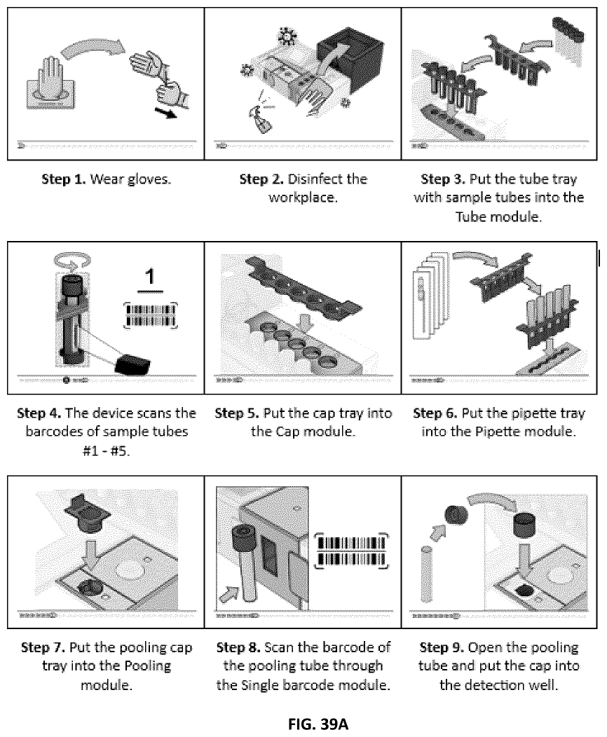

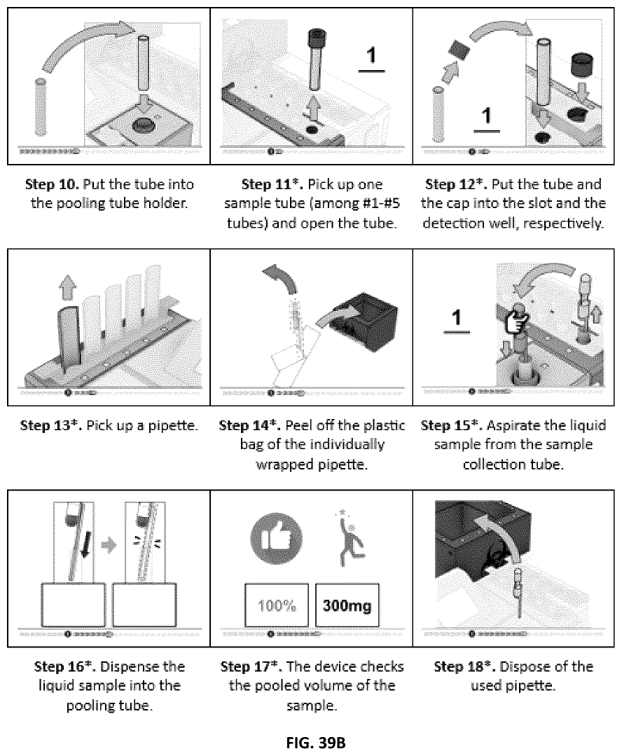

FIG. 37 shows a top view of the single-barcode module 106. The barcode scanner 2620 is fixed to a small plastic structure 2622 extruded from the outer wall 2624 of the single-barcode module 106. The barcode scanner 2620 scans the barcode of the pooling tube 151 when a microcontroller sends a command signal to the scanner 1620; otherwise, the scanner 2620 is in an idle state. The scanner 1620 can scan 1D and 2D codes, and the barcode of the pooling tube 151 contains particular information not to be overwritten by other barcodes. A preferred distance between the scanner 2620 and the window 2610 for scanning is 57 mm, meaning that the scanning distance is secured inside the module, and the scanner 2620 can detect the barcode easily when the pooling tube 151 is close to the window 2610.

The single-barcode module 106 may also be used to choose an operational mode of the instructional point-of-care device 100. As shown in FIG. 38, the single-barcode module 106 may be configured to read these barcodes from cards, paper, or devices such as smart phones. Before starting the sample handling process, the single-barcode module 106 of the device 100 may scan a 2D barcode to choose an operational mode from the training mode for those who are inexperienced in the sample handling procedure of diagnostic workflow and the expert mode for those who are skilled in the handling procedure of the diagnostic process. The instructional point-of-care device 100 may also provide other mechanisms for selection of an operational mode.

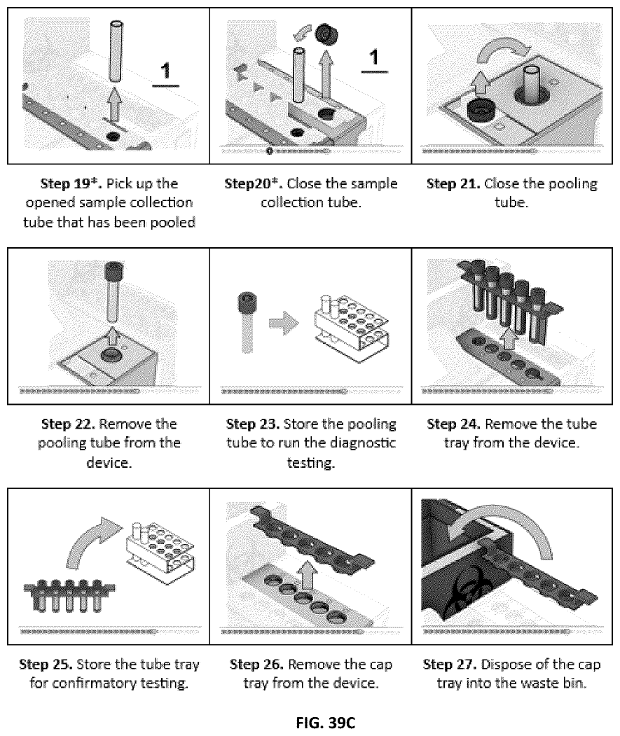

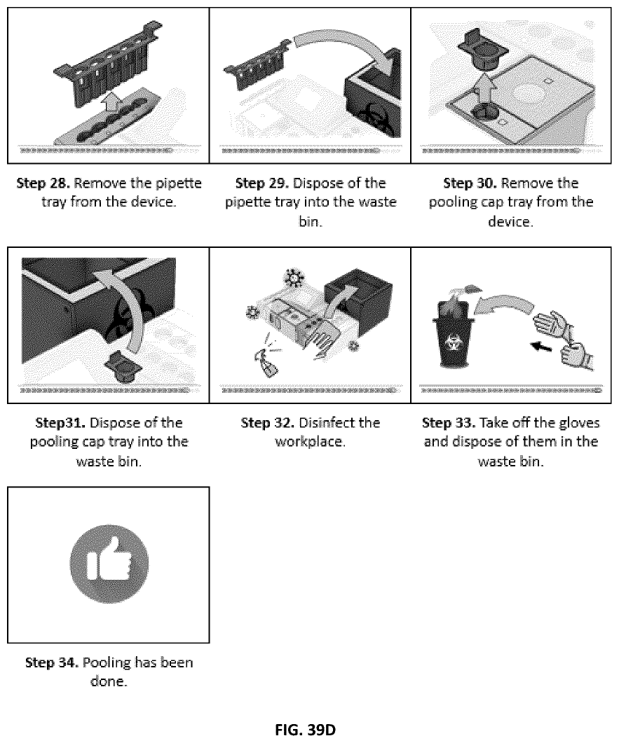

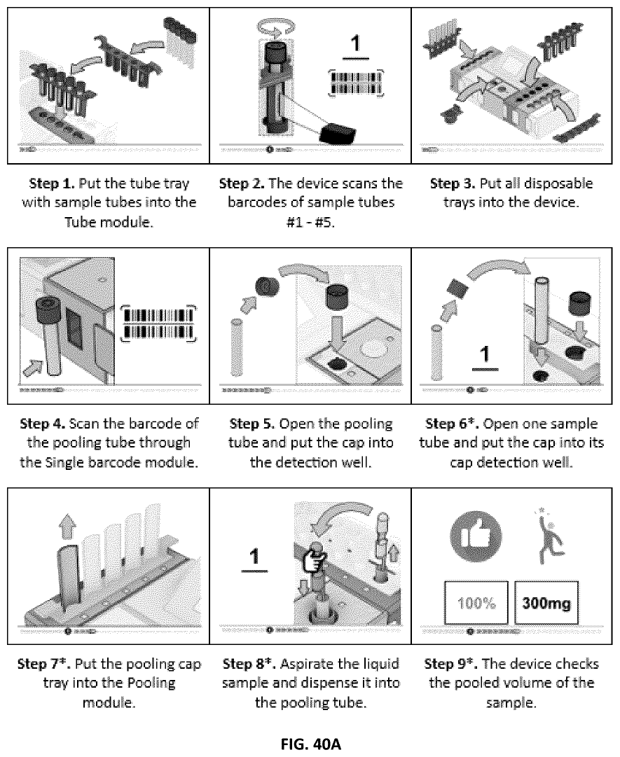

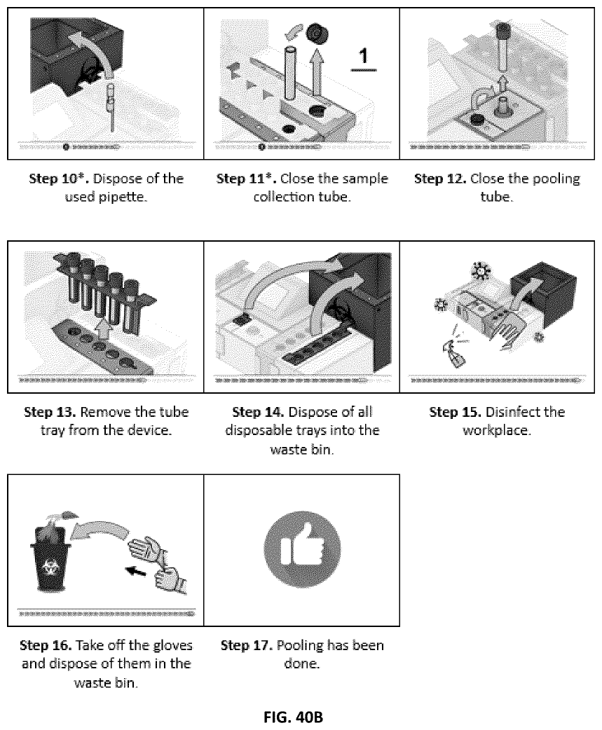

The purpose of the training mode is to teach an unskilled user how to perform the sample handling process properly without mistakes. The training mode may guide the user to do the sample handling process step by step with preventive measures in terms of biosafety, contamination of the workplace, and cross-contamination between samples to samples. For sample pooling, the workflow in the training mode may consist of a total of 34 steps. FIGS. 39A-39D illustrate the steps of the training mode workflow. FIG. 39A illustrates steps 1-9, FIG. 39B illustrates steps 10-18, FIG. 39C illustrates steps 19-27, and FIG. 39D illustrates steps 29-34. Note that steps designated with asterisks (*steps 11-20) will be repeated according to the pool size until all samples are pooled.

The purpose of the expert mode is to help a trained user perform the sample handling process properly without mistakes and accelerate the healthcare workflow in diagnostics. The expert mode may guide the user to do the sample handling process step by step with minimum preventive measures and help the user catch significant issues such as cross-contamination. For sample pooling, the workflow in expert mode may consist of a total of 17 steps. FIGS. 40A and 40B illustrate the steps of the expert mode workflow. FIG. 40A illustrates steps 1-9 and FIG. 40B illustrates steps 10-17. Note that steps designated with asterisks (*steps 6-11) will be repeated according to the pool size until all samples are pooled.

As discussed above, the instructional point-of-care device described above has a modular configuration. However, regardless of whether the device is modular or not, a user may customize the functions of the device according to the logistics of workflow. In other words, some parts or functions of the device could be activated or inactivated in order for better workflow and usability based on the user's preference for sample handling protocols. In this case, the user may maximize the efficiency of sample handling workflow and follow steps different than those illustrated in FIGS. 39A-39D or FIGS. 40A-40B.

During sample testing with the instructional point-of-care device 100, when the pooling tube 151 gets a positive result, its original individual sample tubes 148 should be re-tested to identify individuals' infections. The tube tray 143 delivered with the device may have a symbolic indicator at the side. The information for its symbol is included in the barcode of the tube tray 143, meaning that the device will save the information on internal and external storage units. Containers with barcodes and numeric markers may be delivered with the device 100. When the container is scanned at the end of the pooling workflow, then the coordinates for the storage of the tube tray 143 may be created.

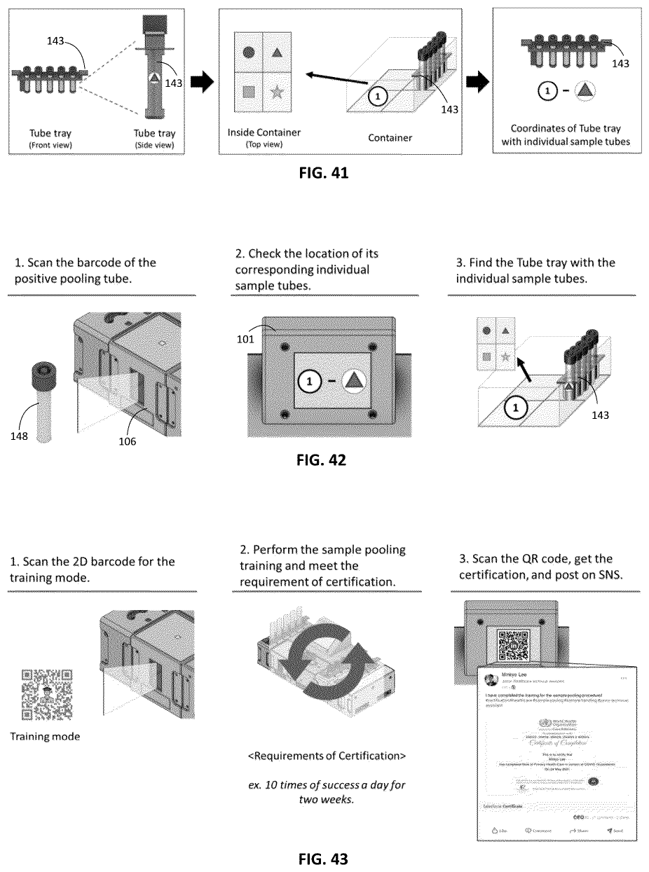

The tube tray 143 with a symbolic marker may be stored in a container that has symbolic markers inside and a numeric marker on its outside wall. The coordinates of the tube tray 143 would be created based on symbolic marker and container number. FIG. 41 illustrates the use of symbolic and numerical markers. In FIG. 41, the first drawing shows a tube tray 143 with a symbolic marker, the second drawing shows how tube trays 143 with differing symbolic markers may be stored in containers designated with numerical markers, and the third drawing shows how an individual tube tray 143 may be designated by its symbolic marker and container numerical marker.

From the combination of numerical indicators and symbolic markers, a user could more easily find individual sample tubes for confirmatory testing when a pooling tube is reported as positive. Before performing the sample handling process, the device may wait for the scanning of barcodes. In this step, the user could scan the barcode of the positive pooling tube. When the device reads the barcode of the pooling tube that has been recorded on external digital storage, the device may show the location of its individual sample tubes. Then, the user could find them easily. FIG. 42 shows a process for locating a sample tray corresponding to a pooling tube with a reported positive test. As shown in FIG. 42, the first step is to scan the barcode of the positive pooling tube with the single-barcode module 106. The second step is to view the display module 101 display, which would show the container number and the tray symbol of the tube tray 143 corresponding to the pooling tube with the positive test. The final step would be to locate the tube tray 143 within its storage container.

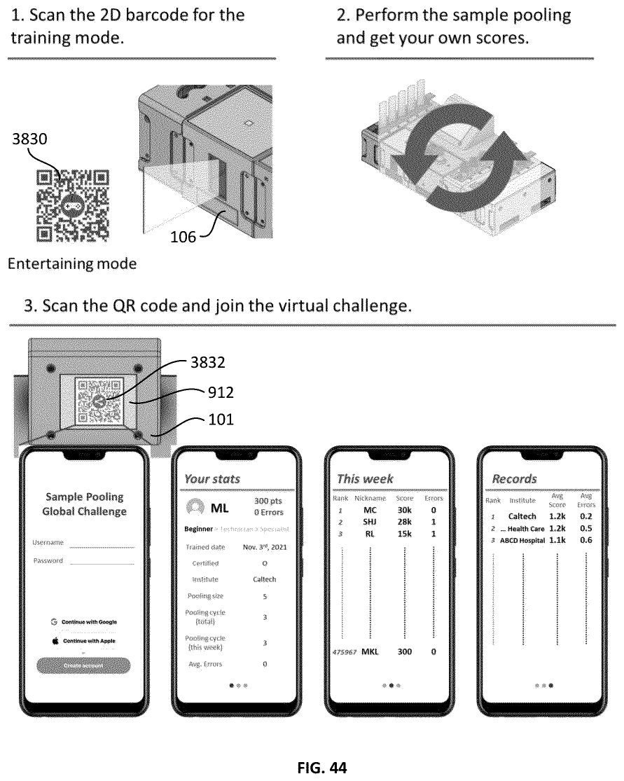

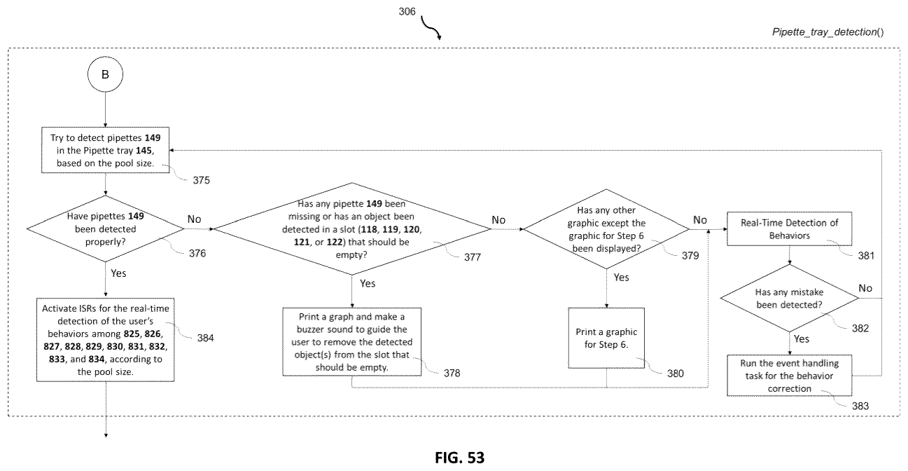

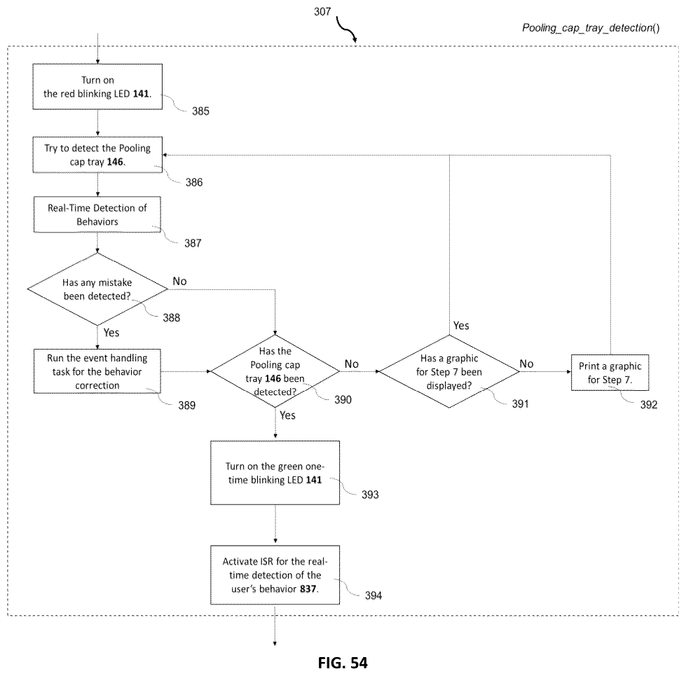

As described above, the instructional point-of-care device provides two different operational modes: training mode and expert mode. When a user scans the barcode for the training mode, the user may participate in the certificate program of training on the sample handling procedures. One possible certification process is shown in FIG. 43. Before performing the sample handling process, the device may wait for the scanning of barcodes. In the first step shown in FIG. 43, a user could scan the barcode for the training mode. In the second step shown in FIG. 53, in the training mode, the user may be able to repeat the dry run of sample pooling procedures without clinical samples. The performance of sample pooling would be recorded on internal and external digital storage. In the final step shown in FIG. 43, when the user meets the requirement for the certificate, the device may provide a barcode that can be scanned by the user for certification. The user may be able to get the certificate and post it on social media platforms (such as LinkedIn™) which may help the user's career.

The instructional point-of-care device may also be configured to allow a user to learn the sample handling process through entertainment content. The device may be able to engage the user to learn the sample handling procedures through the entertainment content of the software. When the user scans the barcode for the entertaining mode before starting the sample handling workflow, the device may provide the entertainment content; for example, the user may get scores of 20 when the user passes biosafety-related steps such as wearing gloves step, disinfection step. The user may also get scores of 300 when the user performs the aspiration and dispense steps successfully. The scores could be saved on internal and external digital storage. After finishing a single sample handling process, the device may provide a 2D barcode to let the user access a mobile software environment for social networks or entertaining platforms where users can share their records with each other on a global scale. In the first step shown in FIG. 44, a user scans a barcode 3830 at the single barcode module 106 for entertaining mode. In the second step shown in FIG. 44, sample pooling steps are performed. In the third step shown in FIG. 44, a QR code 3832 is displayed on the display module 101 display 712. This QR code may then be read and used for a mobile software environment for social networks or entertainment platforms.

When a user accesses the mobile platform, the user may be able to see the user's records for such details including, but not limited to, the following items: (1) an acquired score in the previous single sample handling cycle; (2) an average score for all single sample handling cycles; (3) a total acquired score during a week; (4) a total acquired score; (5) certification; (6) associated institute; (7) a total number of performed sample handling process; (8) a total number of errors that the user has made; (9) an average number of errors per a single sample handling process that the user has made; and (10) parameters of sample handling protocols: pool size, the target volume, etc. For entertainment, the device and mobile platform may provide a shared score or information and engage users through enjoyable challenges or missions for user engagement.

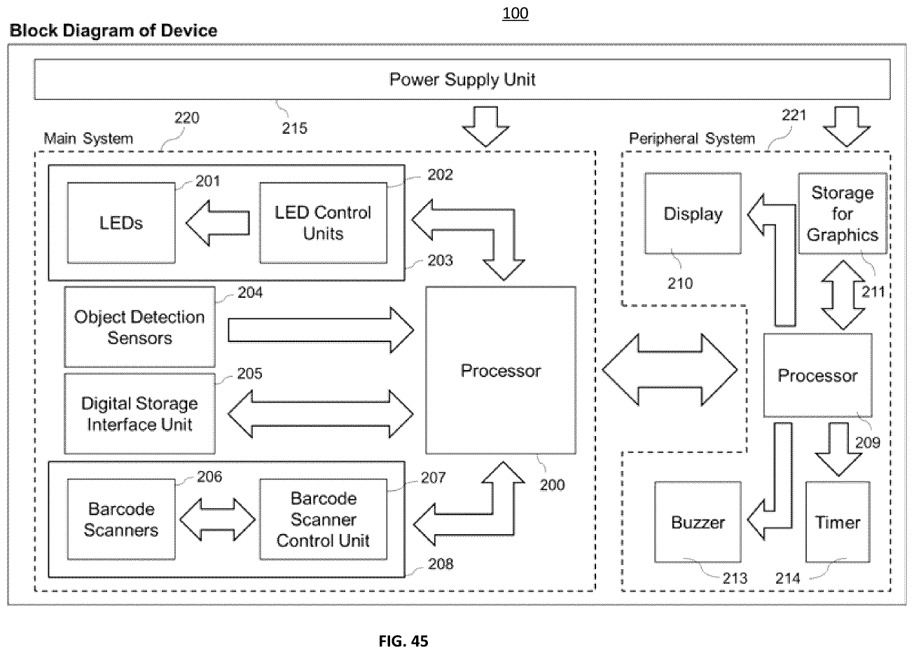

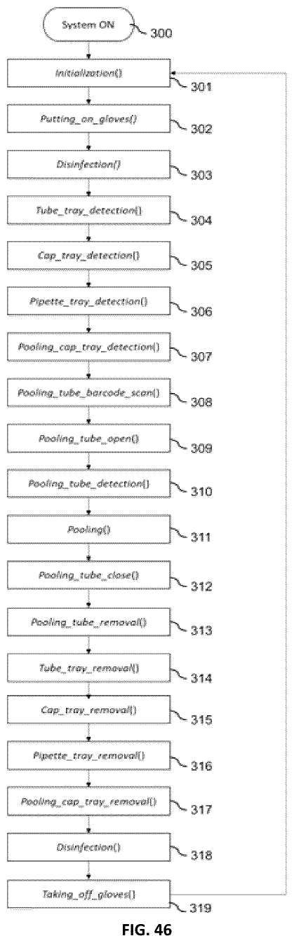

The system block diagram for the architecture of the instructional point-of-care device 100 is shown in FIG. 45. The architecture comprises two systems: a main system 220 and a peripheral system 221. The main system 220 controls the interactive components and operates major operational algorithms for the pooling workflow. The main system 220 performs several functions including, but not limited to, the following functions: (1) controlling LEDs to guide the user on the current workflow status of the device; (2) operating barcode scanners to read barcodes; (3) receiving signals from object detection sensors to detect the user's behaviors; (4) reading and writing data on external digital storage to save medical records regarding the pooling; and (5) communicating with the peripheral system to operate the LCD display, the buzzer, and the timer for the interaction with the user. To perform the listed function, a main processor 200 in the main system 220 is connected to the LED control system 203, object detection sensors 204 (such as IR sensors, lasers, etc.), the digital storage interface unit 205, the barcode scanner control system 208, and the processor 209 of the peripheral system 221.

The LED control system 203 has one or more LED control units 202 to control the LED colors, sequence of illumination, light intensities, and the data transfer to other LED sockets. The main processor 200 sends a control signal(s) to a LED control unit(s) 202 to illuminate a LED socket(s) 201 according to the pooling workflow. In the current system, seven different types of illumination with five different LED colors are used to guide the user to achieve successful pooling. These seven types of illumination are as follows: (1) Red (blinking for 500 milliseconds at one-second intervals)—the red blinking LED indicates that a user should do a task at a place (e.g., a module, a detection well, a slot, etc.) where the red LED is blinking; (2) Green (static from Step 12 to Step 13 in the training mode shown in FIGS. 39A-39C)—the static green LED indicates that a cap 147 of a sample collection tube 148, which is going to be pooled, is successfully loaded in its corresponding detection well of the cap module 103; (3) Green (static)—the static green LED indicates that the sample tube 148 has been already pooled successfully; (4) Green (one-time blinking for 1 second)—the one-time blinking green LED indicates that a step at a place (e.g., a module, a detection well, a slot, etc.) where the red LED was blinking has been performed successfully; (5) Blue (static)—the static blue LED indicates that the current pooling cycle is in progress for a specific sample corresponding to the static blue LED; (6) Pink (blinking for 500 milliseconds at one-second intervals)—the pink blinking LED informs a user that a cap or an object is detected in a cap detection well 113, 114, 115, 116, 117 in the cap module 103 which should have been empty during the cap detection, the blinking LED also indicates that the user should remove the cap or the object from the detection well 113, 114, 115, 116, 117; and (7) Orange (blinking for 500 milliseconds at one-second intervals)—the orange blinking LED indicates that an object, including the tube tray 143, the cap tray 144, the pipette tray 145, the pooling cap tray 146, and the pooling tube 151, should be removed from the device 100.

Object detection sensors 204 are sensors to detect the user's behavior(s) in real-time in order to correct the behavior(s) when the user makes a mistake(s) during the pooling workflow. The object detection sensors 204 include but are not limited to: IR sensors 2203, 2205, 2303, 2305, 2501, 2505, 2511 in the tube module 102, the cap module 102, the pipette module 105, and the pooling module 107. As shown in FIGS. 3 and 4, instructional point-of-care devices may include a waste-bin module 152 for capturing waste products produced during the sample pooling process. When a waste-bin module 152 is included, object detection sensors such as IR light sensors, LED light sensors and/or lasers in the waste module may be used.

The digital storage interface unit 205 is the unit to connect external digital storage, such as an SD card and USB, to the device 100 in order to save medical records regarding the pooling results on the storage.

The barcode scanner control unit 207 is a unit of 8-channel multiplexers for 8-ch TX and 8-ch RX of UART communications between the device and a maximum of 8 barcode scanners 206. In this device, six barcode scanners 206 are used for the barcodes of five sample collection tubes 148 and the tube tray 143.

The peripheral system 221 includes a processor 209 that controls peripheral components only when the processor 209 receives a control signal(s). The peripheral components include, but are not limited to, an LCD display panel 210 to show language-agnostic graphics, a buzzer 213 to make an alarming sound, and a timer 214 to measure time intervals for particular steps. The LCD display panel 210 may comprise the TFT LCD display 712 shown in FIGS. 3 and 4.

The operational software algorithm for the sample pooling workflow in the training mode (shown in FIGS. 39A, 39B, 39C, and 39D) consists of 19 different functional blocks. The system will iterate the whole cycle infinitely. FIG. 46 shows the flow chart for the operational algorithm of the sample pooling workflow in the training mode. Individual portions of the flow chart shown in FIG. 46 are described in additional detail below.

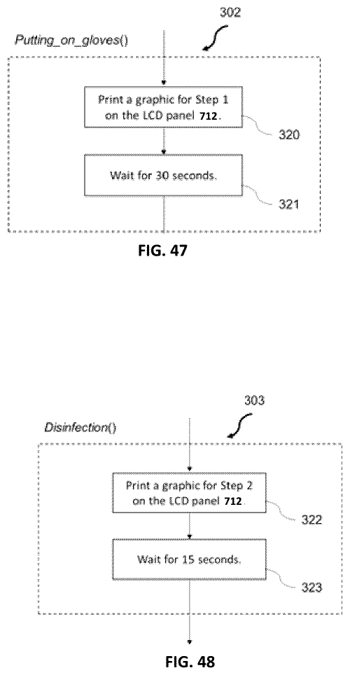

The Putting_on_gloves( ) function 302 is to guide the user to wear gloves properly. FIG. 47 shows the flow chart for the Putting_on_gloves( ) function 302. The device 100 prints a graphic on the TFT LCD 712 for the first block 320 (step 1 of the sample pooling workflow) and waits 30 seconds for the wearing of gloves as shown by the second block 321.

The Disinfection( ) function 303 is to guide the user to disinfect the device and its workplace by using 70% ethanol properly. The device prints a graphic on the TFT LCD 712 for the first block 322 (step 2 of the sample pooling workflow) and waits 15 seconds for the disinfection as shown by the second block 323. When the waste-bin module 152 is available, the device waits until the user disposes of wipes or towels, used for cleaning, into the disposal container 162. When the device detects the disposal action from the user, the LEDs 153, 154, 155, 156, 157, 158, 159, 160, 161 will illuminate the green color for 1 second and are turned off. The Disinfection( ) function 303 will be implemented again between the steps of Pooling_cap_tray_removal( ) and Taking_off_gloves( ).

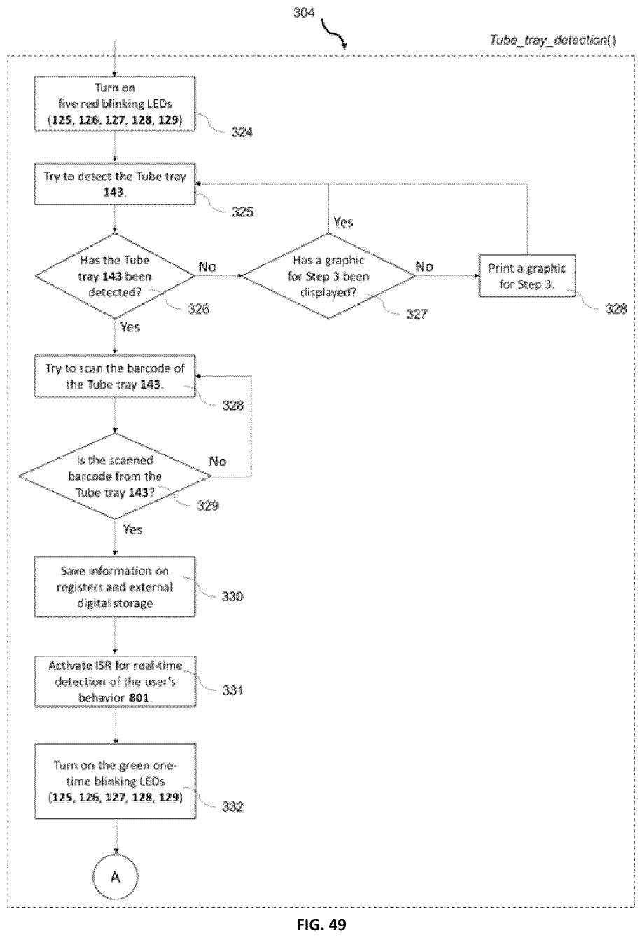

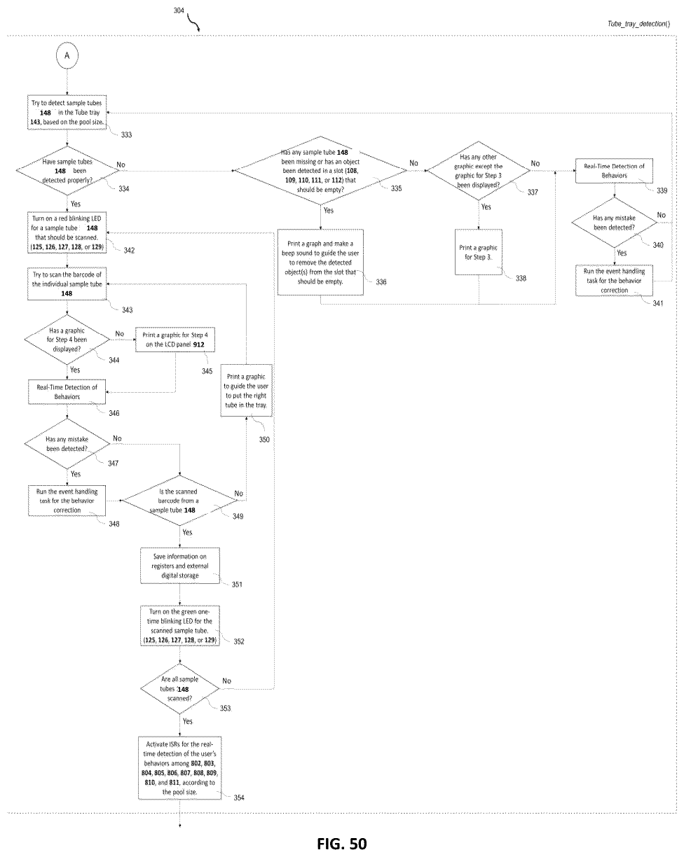

The Tube_tray_detection( ) function 304 is to detect whether the user loads the tube tray into the tube module properly. FIG. 49 shows a first portion of a flow chart for the Tube_tray_detection( ) function 304 and FIG. 50 shows a second portion of the flow chart for the Tube_tray_detection( ) function 304.

The device 100 turns on all five tube module receiving slot status LEDs 125, 126, 127, 128, 129 of the tube module 102 blinking red at one-second intervals as shown by block 324 of FIG. 49. If the user has not put the tube tray 143 into the device yet as shown at block 326, the device 100 prints a graphic on the TFT LCD 712 as shown by block 328 (for step 3 of the sample pooling workflow). The graphic informs that the user should put the tube tray 143 with individual sample collection tubes 148 into the device. At the same time, the device turns on the barcode scanner #1, which waits for the barcode of the tube tray 143. When the tube tray 143 with sample tubes 148 is loaded into the device as shown at block 326, scanner #1 scans the barcode of the tube tray 143 and reads information as shown at block 328. The information includes, but is not limited to, the following details; (1) a character indicating the general tube tray; (2) unique code number for individual tube tray; (3) coordinates of Tube Tray; (4) pool size that has been determined by the sample pooling strategy of a user's laboratory; (5) target volume (or weight) of sample from each sample tube; (6) sample type: liquid or solid; transport medium, saliva, serum, urine, blood, sperm, or stool.

From the scanned barcode, the device 100 seeks an indicator for the tube tray 143 to confirm that the scanned barcode is the barcode of the tube tray 143 as shown by block 329. If the scanner reads a wrong barcode, the scanner tries to rescan a barcode. When scanning the barcode of the tube tray 143 correctly, the device turns off scanner #1, saves the information on register buffers, and writes the information on a portable digital storage unit (e.g., SD card, USB, etc.) as shown by block 330. Then, the device 100 activates an interrupt routine service (ISR) for the IR sensor 331 for the tube tray 143 as shown by block 331. Then all five LEDs 125, 126, 127, 128, 129 illuminate the green color for 1 second and are turned off as shown by block 332.

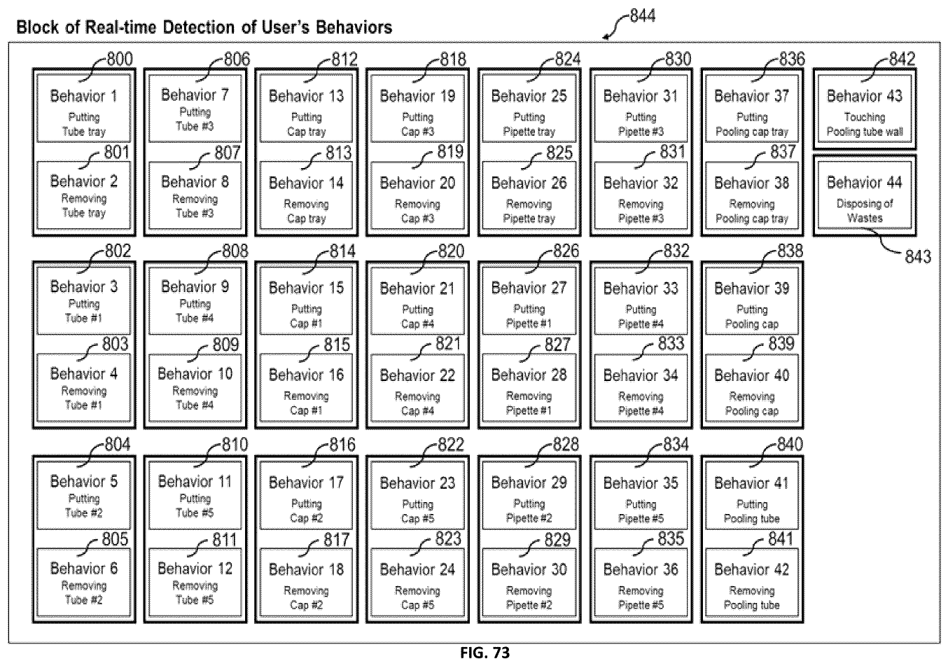

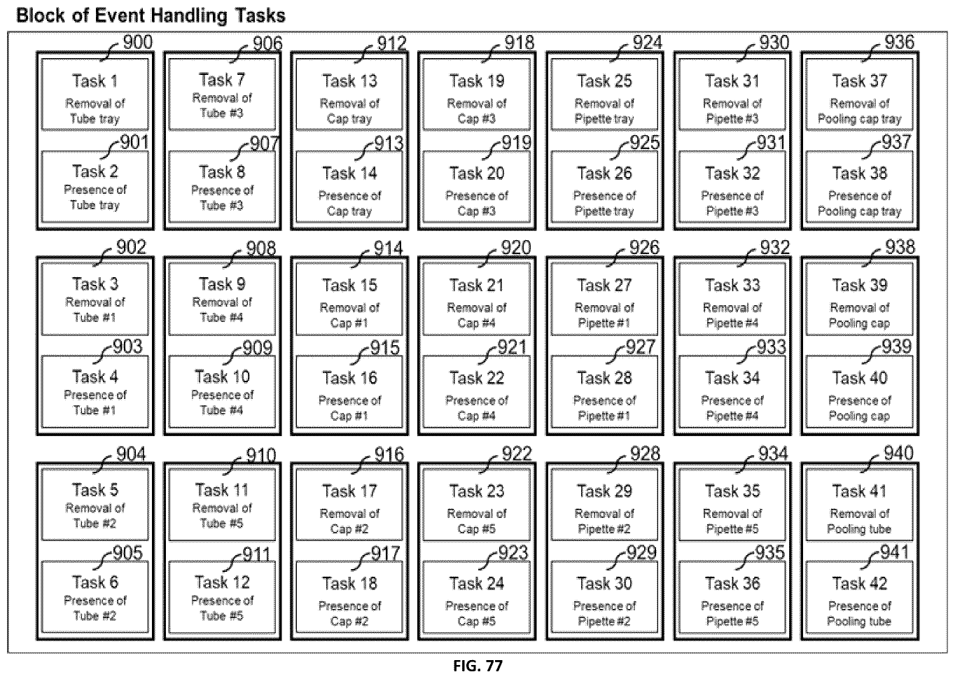

The ISR is edge-triggered by the IR sensor anytime during the device operation until the inactivation of the ISR. The activation of ISR for the sensor for the tube tray 143 allows the real-time detection of the user's behavior of removing the tube tray 143 from the module 102 (see FIG. 73 for real-time detection of user's behaviors). When the behavior is detected, the device calls its corresponding event handling task (see FIG. 77 for event handling tasks).

The next step is to detect the presence and absence of sample collection tubes 148 inside the tube tray 143, according to pool size as shown by block 333 in FIG. 50. For example, if the pool size is 3, the device detects the presence of sample collection tubes 148 at slots #1, #2, and #3, 108, 109, 110 of the tube module 102 and the absence of sample collection tubes (or any objects) at slots #4 and #5 111, 112 of tube module 102 as shown by block 334. When the device 100 could not find a tube(s) 148 in a slot(s) among slots #1, #2, and #3 108, 109, 110, or when the device 100 finds a tube(s) or any object in a slot(s) among slots #4 and #5 111, 112, the device 100 prints a graphic on the TFT LCD 712 showing the absence of a tube(s) at the slot(s) and makes a beep sound for 2 seconds at one-second intervals until the all tubes 148 exist in the slots properly as shown by block 336.

The final step is to scan the barcode of each sample collection tube 148 according to the pool size as show by block 343. The device 100 tries to read the barcode of sample tube #1 through the barcode scanner #2 with the corresponding red LED 125 blinking at one-second intervals as shown by block 343. At the same time, the device 100 prints a graphic(s) on the TFT LCD 712 as shown by block 3454 (for Step 4 of the sample pooling workflow). The user should align the barcode with the scanner before or during the scanning to make the scanning successful. The barcode of each sample collection tube 148 contains information including, but not limited to, the following details: (1) a character indicating the general sample collection tubes; (2) unique code number for individual sample collection tubes, and (3) sample type: liquid or solid; transport medium, saliva, serum, urine, blood, sperm, or stool. From the scanned barcode, the device 100 seeks an indicator for general sample collection tubes 148 to confirm that the scanned barcode is one of the barcodes of sample collection tubes as shown by block 349.

The confirmation prevents the misuse of other tubes/objects and the reuse of sample collection tubes. If the scanner reads a wrong barcode, the device 100 prints a graphic on the TFT LCD 712 showing that the scanned barcode has come from unknown tubes or objects and tries to rescan a barcode. If the scanner reads a used barcode, the device prints a graphic on the TFT LCD 712 showing that the sample tube has been already used before and tries to rescan a barcode.

When the barcode of sample tube #1 is scanned correctly, the device turns off scanner #2, saves the information on register buffers, and writes the information on the portable digital storage unit (e.g., SD card, USB, etc.) as shown by block 351. At the same time, the corresponding red LED socket 125 turns into the green LED for 1 second and is turned off as shown by block 352. Similarly, the device reads the barcodes of sample tubes according to the pool size as shown by blocks 343, 349, 350, 351, 352, 353. When all sample tubes 148 with the tube tray 143 are correctly loaded and scanned properly as shown by block 353, the device 100 activates the ISRs for each IR sensor for each slot of the Tube module as shown by block 354.

The ISRs are edge-triggered by IR sensors anytime during the device operation until the inactivation of the ISRs. The activation of the ISRs for the sensors for all slots of the tube module 102 allows the real-time detection of the user's behaviors of putting an object into the Tube module and removing sample tubes from the Tube module (see FIG. 73 for real-time detection of user's behaviors). When one or more of the behaviors are detected, the device 100 calls their corresponding event handling tasks (see FIG. 77 for event handling tasks).

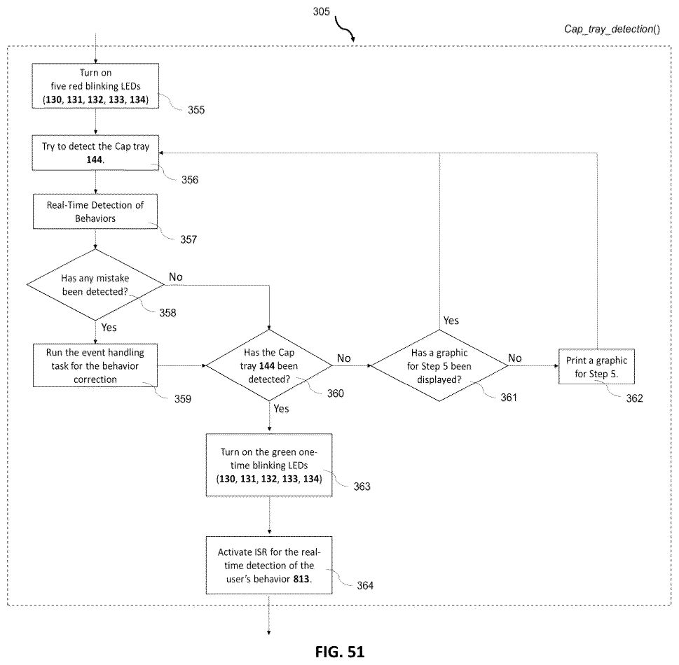

The Cap_tray_detection( ) function 305 is to detect whether the user loads the cap tray 144 into the cap module 103 properly. FIG. 51 shows the flow chart for the Cap_tray_detection( ) function 305. The device 100 turns on all five cap module detection well status LEDs 130, 131, 132, 133, 134 of the cap module 103 blinking red at one-second intervals as shown by block 355. If the user has not put the cap tray 144 into the device yet as shown by block 360, the device 100 prints a graphic on the TFT LCD 712 as shown by block 361 (for Step 5 of the sample pooling workflow) to let a user install the cap tray 144 and waits for the loading of the cap tray 144. When the cap tray 144 is loaded properly as shown by block 360, the five red LEDs 130, 131, 132, 133, 134 turn into the green LEDs for 1 second and are turned off as shown by block 363. Then the device 100 activates the interrupt routine service (ISR) for the IR sensor for the cap tray 144 as shown by block 364.

The ISR is edge-triggered by the IR sensor anytime during the device operation until the inactivation of the ISR. The activation of ISR for the sensor for the cap tray 144 allows the real-time detection of the user's behavior of removing the cap tray 144 from the Cap module 103 (see FIG. 73 for Real-Time Detection of User's Behaviors). When the behavior is detected, the device calls its corresponding event handling task (see FIG. 77 for Event Handling Tasks).

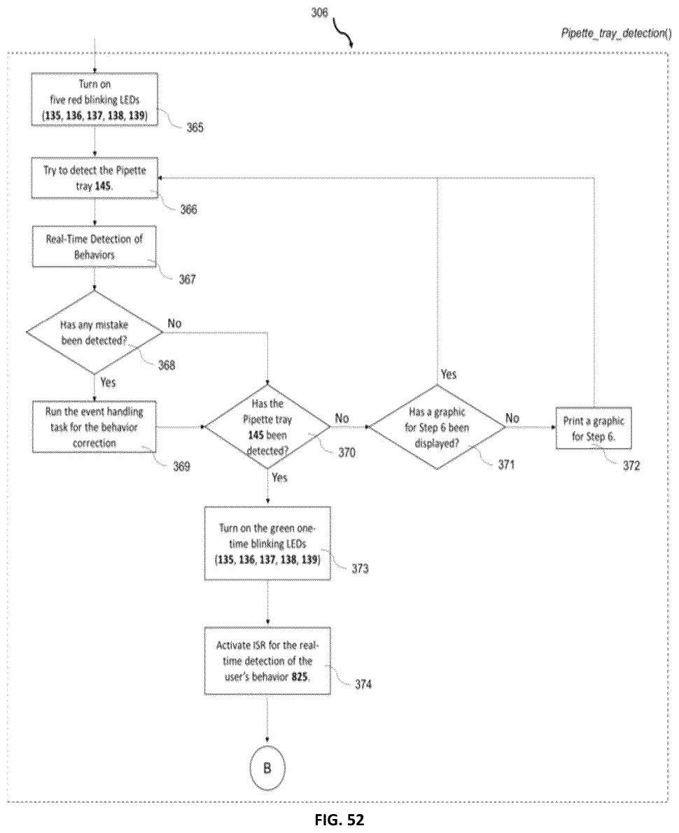

The Pipette_tray_detection( ) function 306 is to detect whether the user loads the pipette tray 145 into the pipette module 105 properly. FIG. 52 shows a first portion of the flow chart for the Pipette_tray_detection( ) function 306 and FIG. 53 shows a second portion of the flow chart for the Pipette_tray_detection( ) function 306.

The device 100 turns on all five pipette module receiving slot status LEDs 135, 136, 137, 138, 139 of the pipette module 105 blinking red at one-second intervals as shown in block 365 of FIG. 52. If the user has not put the pipette tray 145 into the device yet as shown by block 370, the device 100 prints a graphic on the TFT LCD 712 as shown by block 371 (for Step 6 of the sample pooling workflow) to let a user install the pipette tray 145 into the device 100. When the pipette tray 145 is loaded properly as shown by block 370, the five red LEDs 135, 136, 137, 138, 139 turn into the green LEDs for 1 second and are turned off as shown by block 373. Then the device activates the interrupt routine service (ISR) for the IR sensor for the pipette tray 145 as shown by block 374.

The ISR is edge-triggered by the IR sensor anytime during the device operation until the inactivation of the ISR. The activation of ISR for the sensor for the pipette tray 145 allows the real-time detection of the user's behavior of removing the pipette tray 145 from the pipette module 105 (see FIG. 73 for Real-Time Detection of User's Behaviors). When the behavior is detected, the device calls its corresponding event handling task (see FIG. 77 for Event Handling Tasks).

The next step is to detect the presence and absence of individually packaged exact volume pipettes 149 inside the pipette tray 145, according to pool size as shown by block 375 of FIG. 53. For example, if the pool size is 3, the device detects the presence of pipettes 149 at slots #1, #2, and #3 118, 119, 120 of the pipette module 105 and the absence of pipettes (or any objects) at slots #4 and #5 121, 122 of pipette module 105. When the device 100 could not find a pipette(s) in a slot(s) among slots #1, #2, and #3 118, 119, 120, or when the device 100 finds a pipette(s) or any object(s) in a slot(s) among slots #4 and #5 121, 122, the device prints a graphic on the TFT LCD 712 showing that pipettes 149 are not loaded properly and makes a beep sound for 2 seconds at one-second intervals until all pipettes 149 exist in their slots properly as shown by block 378. When all pipettes 149 with the pipette tray 145 are correctly loaded properly as shown by block 376, the device 100 activates the ISRs for each IR sensor for each slot of the pipette module 105 as shown by block 384.

The ISRs are edge-triggered by IR sensors anytime during the device operation until the inactivation of the ISRs. The activation of the ISRs for the sensors for all slots 118, 119, 120, 121, 122 of the pipette module 105 allows the real-time detection of the user's behaviors of putting an object into the pipette module 105 and removing pipettes 149 from the pipette module 105 (see FIG. 73 for Real-Time Detection of User's Behaviors). When one or more of the behaviors are detected, the device calls their corresponding event handling tasks (see FIG. 77 for Event Handling Tasks).

The Pooling_cap_tray_detection( ) function 307 is to detect whether the user puts the Pooling cap tray into the cap detection well of the Pooling module properly. FIG. 54 shows the flow chart for the Pooling_cap_tray_detection( ) function 307.

The device 100 turns on the pooling cap detection well status LED 141 close to the detection well 123 for a pooling cap blinking red at one-second intervals. If the user has not put the pooling cap tray 146 into the device as shown by block 390 of FIG. 54, the device 100 prints a graphic on the TFT LCD 712 for Step 7 of the sample pooling workflow as shown by block 392 to let a user put the pooling cap tray 146 into the device 100. When the pooling cap tray 146 is loaded properly as shown by block 390, the LED 141 turns from red into green for 1 second and is turned off as shown by block 393. Then the device 100 activates the interrupt routine service (ISR) for the IR sensor for the pooling cap tray 146 as shown by block 394.

The ISR is edge-triggered by the IR sensor anytime during the device operation until the inactivation of the ISR. The activation of ISR for the sensor for the pooling cap tray 146 allows the real-time detection of the user's behavior of removing the pooling cap tray 146 from the pooling module 107 (see FIG. 73 for Real-Time Detection of User's Behaviors). When the behavior is detected, the device calls its corresponding event handling task (see FIG. 77 for Event Handling Tasks).

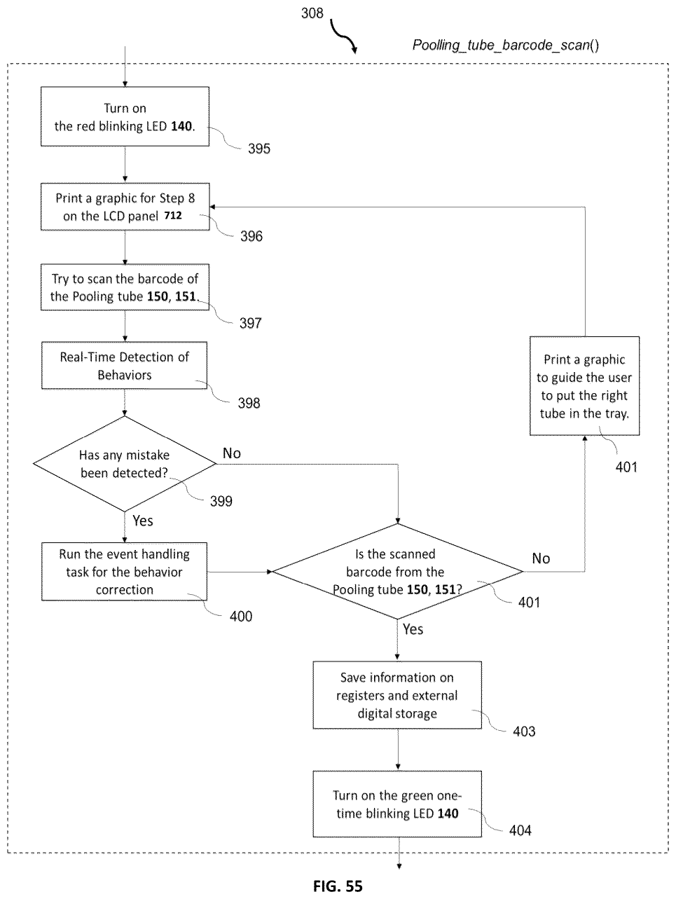

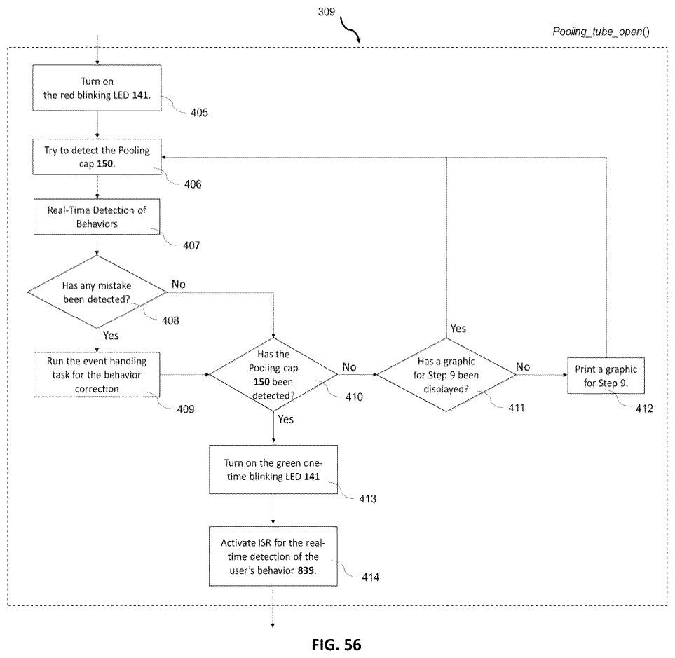

The Pooling_tube_barcode_scan( ) function 308 is to read the barcode of the pooling tube 151 by the barcode scanner of the single-barcode module 106. FIG. 55 shows the flow chart for the Pooling_tube_barcode_scan( ) function 308.

The device 100 turns on the barcode module status LED 140 of the single-barcode module 106 at one-second intervals as shown by block 395 of FIG. 55. Then, the device 100 prints a graphic on the TFT LCD 712 for Step 8 of the sample pooling workflow to let a user scan the Pooling tube 150, 151. At the same time, the device turns on the barcode scanner inside the single-barcode module 106 and waits for the scanning of the barcode of the pooling tube 150. The barcode of the pooling tube 150 contains information including, but not limited to, the following details: (1) a character indicating the general Pooling tube; (2) unique code number for the pooling tube; and (3) sample type: liquid or solid, and transport medium, saliva, serum, urine, blood, sperm, or stool. When a barcode is scanned, the device seeks an indicator for the pooling tube 150 to confirm that the scanned barcode is the barcode of the pooling tube 150 as shown by block 401.

The confirmation enables the prevention of the misuse of other tubes/objects and the reuse of pooling tubes 150. If the scanner reads a wrong barcode, the device prints a graphic on the TFT LCD 712 showing that the scanned barcode has come from unknown tubes or objects and tries to rescan a barcode as shown by block 401. If the scanner reads a used barcode, the device 100 prints a graphic on the TFT LCD 712 showing that the pooling tube 140 has been used before and tries to rescan a barcode as shown by block 401.

When the barcode of the new pooling tube 150 is scanned correctly as shown by block 402, the device 100 turns off the scanner, saves the information on register buffers, and writes the information on the portable digital storage unit (e.g., SD card, USB, etc.) as shown by block 403. Then the barcode module status LED 140 turns green for 1 second and is turned off as shown by block 404.

The Pooling_tube_open( ) function 309 is to detect whether the user opens the pooling tube 150 and puts its cap 151 into the cap detection well 123 of the pooling module 107 properly. FIG. 56 shows the flow chart for the Pooling_tube_open( ) function 309.

The device 100 turns the pooling cap detection well status LED 141 for the detection well 123 red at one-second intervals as shown by block 405 of FIG. 56. If the user has not put the pooling cap 150 into the device yet as shown by block 410, the device 100 prints a graphic on the TFT LCD 712 for Step 9 of the sample pooling workflow as shown by block 411 to let a user open the pooling tube 151 and load its cap 150 into the detection well 123 of the pooling module 107. When the pooling cap 150 is loaded into the well 123 successfully as shown by block 410, the device 100 turns off pooling cap detection well status LED 141 as shown by block 413 and activates the interrupt routine service (ISR) for the IR sensor for the pooling cap 150 as shown by block 414.

The ISR is edge-triggered by the IR sensor anytime during the device operation until the inactivation of the ISR. The activation of ISR for the sensor for the pooling cap 150 allows the real-time detection of the user's behavior of removing the pooling cap 150 from the pooling cap detection well 123 of the pooling module 107 (see FIG. 73 for Real-Time Detection of User's Behaviors). When the behavior is detected, the device calls its corresponding event handling task (see FIG. 77 for Event Handling Tasks).

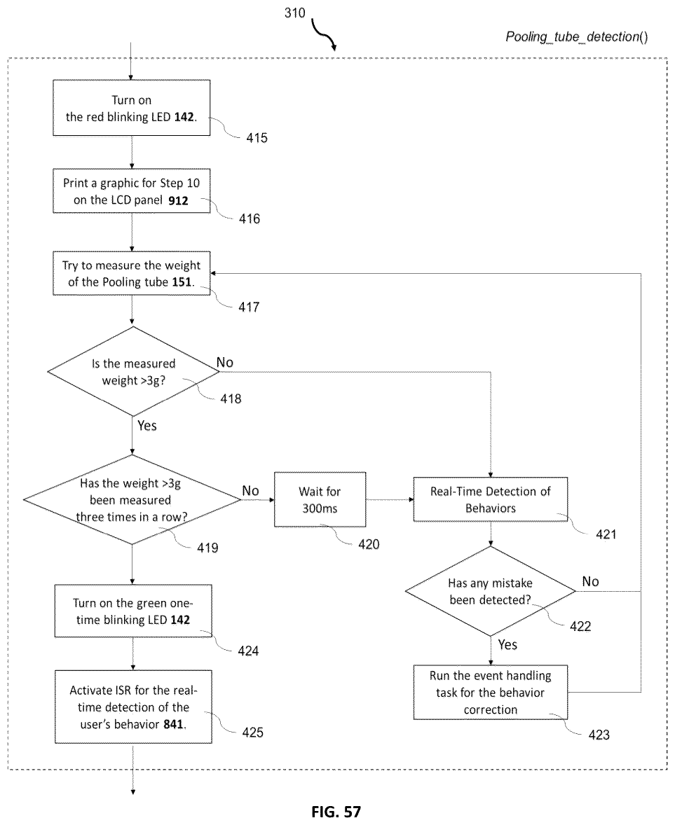

The Pooling_tube_detection( ) function 310 is to detect whether a user loads the pooling tube 151 into the pooling tube holder 124 of the pooling module 107 successfully. FIG. 57 shows the flow chart for the Pooling_tube_detection( ) function 310.

The device 100 turns on the pooling tube holder status LED 142 for the pooling tube holder 124 to red and waits for the loading of the pooling tube 151 as shown by block 415 of FIG. 57. Then, the device 100 prints a graphic on the TFT LCD 712 for Step 10 of the sample pooling workflow as shown by block 416 to guide a user to put the pooling tube 151 into the pooling tube holder 124. Since the weighing scale has been tared, the device 100 detects the loading of the pooling tube 151 by measuring the weight of, for example, >3 g, which is 80% of the pooling tube weight. The system may be configured to detect other weights, depending upon the pooling tubes.

When the pooling tube 151 is loaded into the device 100 successfully as shown by block 419, the pooling tube holder status LED 142 turns from red into green for 1 second and is turned off as shown by block 424. Then the device activates the real-time detection of the user's behavior as shown by block 425 of removing the pooling tube 151 from the pooling module 107 (see FIG. 73 for Real-Time Detection of User's Behaviors). When the behavior is detected, the device calls its corresponding task (see FIG. 77 for Event Handling Tasks).

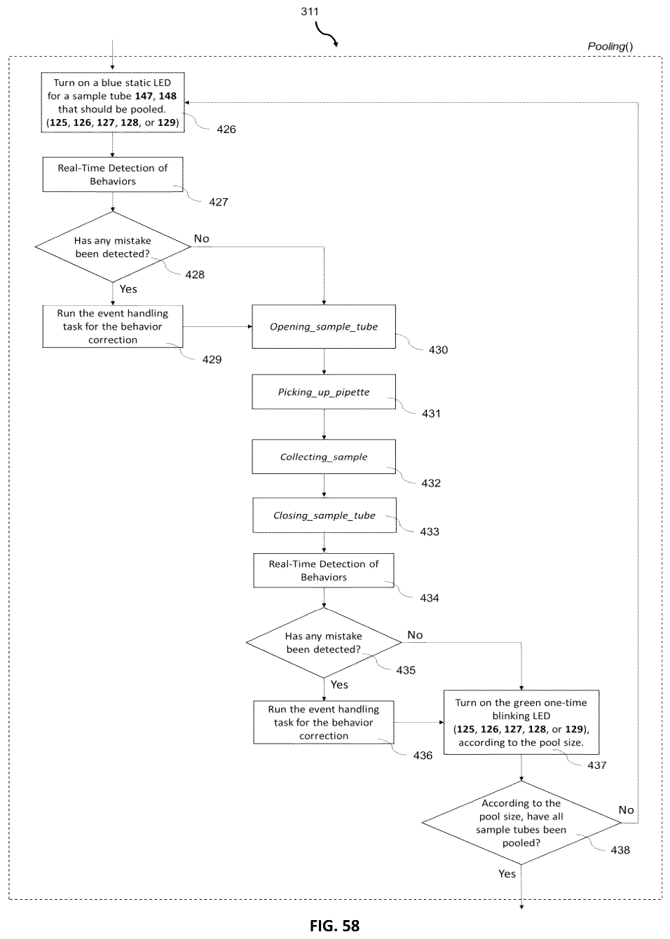

In the Pooling( ) function 311, the device 100 performs a pooling cycle that is repeated according to the pool size. FIG. 58 shows the flow chart for the Pooling( ) function 311. In every pooling cycle, the Pooling( ) function 311 consists of the following four tasks: the Opening_sample_tube task 430; the Picking_up_pipette task 431; the Collecting_sample task 432; and the Closing_sample_tube task 433 as shown in FIG. 58.

The Opening_sample_tube task 430 is to guide a user to open the correct sample collection tube in sequence. The critical aspect of the task is to prevent mistakes in opening the wrong sample tube. For example, the device 100 should prevent the case of the opening of sample tube #2 even though sample tube #3 should be opened. In this regard, the device 100 requires a user to perform the following procedure:
(1) Pick up the correct sample tube 148 with cap 147 from the tube module 102.
(2) Open the sample tube 148 by removing the cap 147.
(3) Put the opened tube 148 and its cap 147 into the corresponding slot 108, 109, 110, 111, 112 of the tube module 102 and the corresponding detection well 113, 114, 115, 116, 117 of the cap module 103, respectively.
(4) Align the barcode of the tube 148 with the corresponding barcode scanner to allow the device 100 to scan the barcode of the sample tube 148 successfully.

The requirement of picking up the correct sample tube 148 guarantees that the user has the right tube in hand. From the guaranteed behavior, it can be assumed that the following object detected in the well of the cap module 103 is the cap 147 of the right tube 148. The barcode scanning performed at the end could be a double-check tool to ensure that the user has opened the right tube 148 and put it back into the device 100.

The Picking_up_pipette task 431 is to guide a user to pick up the correct pipette 149 in sequence. The critical aspect of the task is to prevent mistakes in picking up the wrong pipette 149. For example, the device 100 should prevent the case when the user picks up pipette #2 even though sample tube #1 should be taken. The ISRs of IR sensors for all slots 118, 119, 120, 121, 122 of the pipette module 105 can detect the mistakes.

The Collecting_sample task 432 is to guide a user to pool the individual sample liquid properly into the pooling tube 151. The task consists of two steps: aspiration and dispense. For the aspiration step, the device 100 could not detect the behavior of aspiration. Instead, the device detects the dispense by measuring the collected weight of the sample.

A critical aspect during the collection is to prevent biosafety issues, including the case when a user accidentally dispenses some amount of the sample outside the pooling tube 151 and contaminates the workplace. Another important aspect is to prevent the recollection of the sample from the sample tube 148 by the used transfer pipette 149. The recollection can cause cross-contamination between the used pipette 149, which has been exposed to the pooled liquid, and the pure liquid sample in the tube 148. Therefore, the device requires a user to perform the following procedure:
(1) Aspirate the sample from the tube 148.
(2) Touch the inner wall of the pooling tube 151 by the pipette tip.
(3) Keep the pipette tip in contact with the pooling tube wall and dispense the sample from the pipette 149.
(4) Dispose of the used pipette 149.

The requirement that the pipette tip remains in contact with the pooling tube wall guarantees that the pipette tip is inside the tube 151. Thereby, the device 100 can prevent biosafety issues during the dispense of the sample. In addition, the provided pipettes 149 are the exact volume transfer pipettes, meaning that the user may not be tempted to reuse the pipette 149. To prevent the recollection of the sample from the sample tube 148, the device 100 should inform the user that the liquid sample inside the pipette 149 must be dispensed in one go, and the used pipette 149 must be discarded after dispensing.

The Closing_sample_tube task 433 is to guide a user to close the cap 147 of the pooled sample tube 148. For this task, the device detects the absence of the cap 147 from the detection well 113, 114, 115, 116, 117 of the cap module 103.

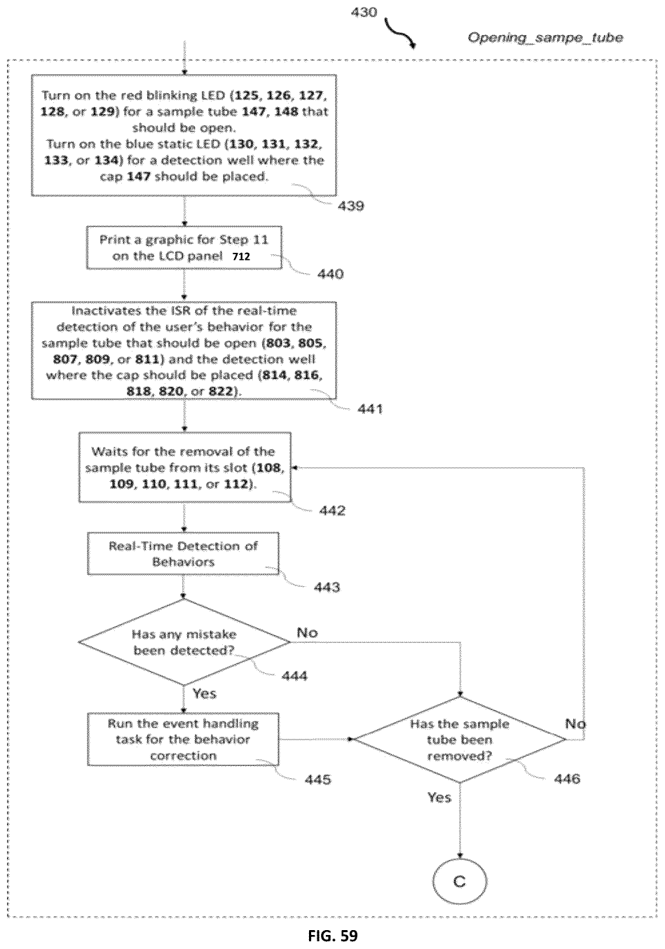

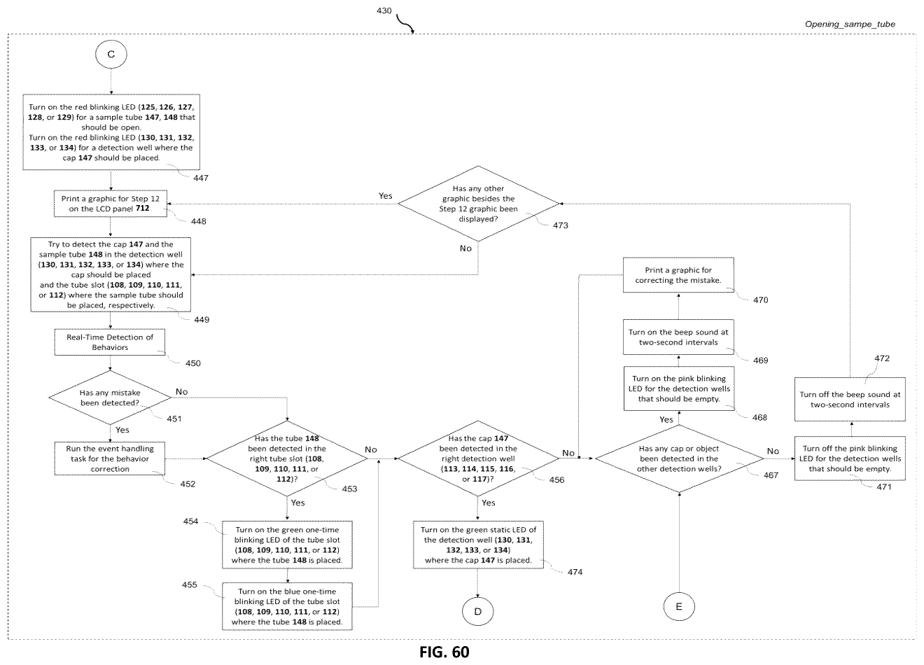

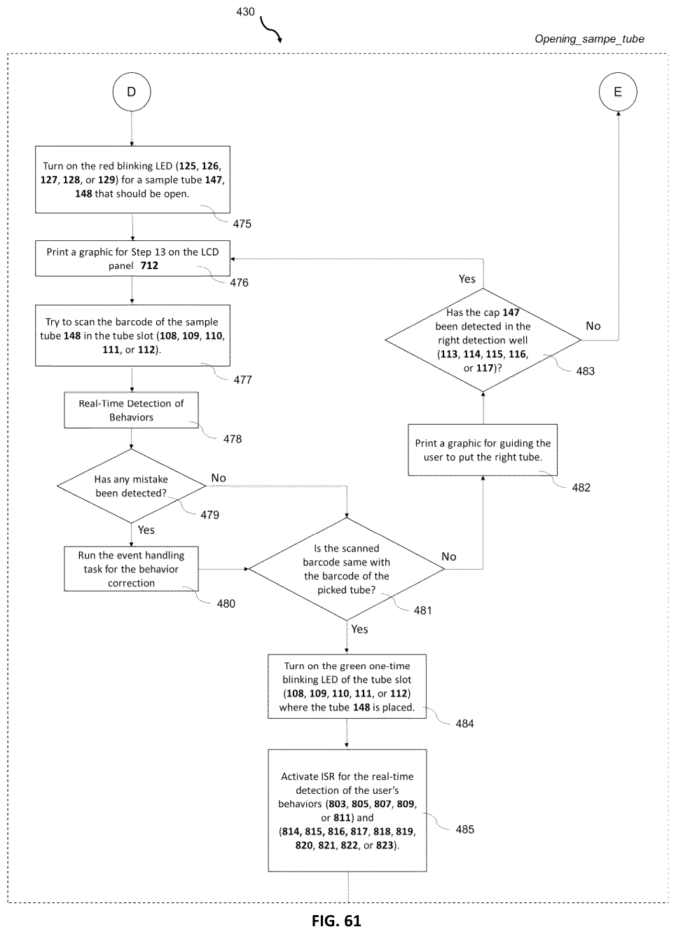

FIG. 59, FIG. 60, and FIG. 61 show portions of the flow chart for the Opening_sample_tube task 430. As shown by block 439 of FIG. 59, for the first task 430—Opening_sample_tube—the device 100 turns one of the tube module receiving slot status LEDs 125, 126, 127, 128, 129 of the corresponding slot 108, 109, 110, 111, 112 of the tube module 102 to blinking red. At the same time, the device 100 turns one of the cap module detection well status LEDs 130, 131, 132, 133, 134 of the corresponding slot 113, 114, 115, 116, 117 of the pap module 103 to static blue. Then, the device 100 prints a graphic on the TFT LCD 712 for Step 11 of the sample pooling workflow to guide the user to open the correct sample tube 148 with cap 147 as shown by block 440. Then the device 100 waits for the removal of the sample tube 148 from the device 100. Note that the device inactivates the real-time detection of the user's behavior of removing the sample tube 148 from the tube module 102 and putting its cap 147 into the cap module 103 as shown by block 441 (see FIG. 73 for Real-Time Detection of User's Behaviors).

When the device 100 detects by its IR sensor that the tube 148 has been removed as shown by block 446, the device turns one of the tube module receiving slot status LEDs 125, 126, 127, 128, 129 and one of the cap module detection well status LEDs 129; 130, 131, 132, 133, 134 for the corresponding slot 108, 109, 110, 111, 112 and the corresponding well 113, 114, 115, 116, 117 to blinking red as shown by block 447 of FIG. 60. At the same time, the device 100 prints a graphic on the TFT LCD 712 for Step 12 of the sample pooling workflow to guide a user to put the opened tube 148 and its cap 147 into the corresponding slot 108, 109, 110, 111, 112 of the tube module 102 and the corresponding detection well 113, 114, 115, 116, 117 of the cap module 103, respectively, as shown by block 448. Then the device 100 waits for the presence of the sample tube 148 and its cap 147 in the tube module 102 and the cap module 103, respectively. When the device 100 detects by its IR sensors that the tube 148 and its cap 147 are present in the device as shown by blocks 453, 456, the cap module detection well status LED 130, 131, 132, 133, 134 of the detection well 113, 114, 115, 116, 117 of the cap module 107 turns from red into green when Step 13 is done as shown by block 474.

Next, the device 100 prints a graphic on the TFT LCD 712 for Step 13 of the sample pooling workflow as shown by block 476 as shown in FIG. 61. In this step, the device tries to read the barcode of the sample tube 148 to confirm that the object is the tube that had been removed as shown by block 477. Until the barcode of the tube 148 is scanned successfully as shown by block 481, the tube module receiving slot status LED 125, 126, 127, 128, 129 of the slot 108, 109, 110, 111, 112 of the tube module 102 is blinking red as shown by block 475. To make the scanning successful, the user may have to align the barcode of the tube 148 with the corresponding barcode scanner. When the barcode of the sample tube is scanned as shown by block 481, the tube module receiving slot status LED 125, 126, 127, 128, 129 of the slot 108, 109, 110, 111, 112 of the tube module turns from red into green for 1 second and is turned off as shown by block 484 with a corresponding cap module detection well status LED 129; 130, 131, 132, 133, 134 of the cap module 103 also going green. Then, the device 100 activates the interrupt routine services (ISR) for the IR sensors for the sample tube 148 and the cap 147 of the sample tube that is going to be pooled as shown by block 485.

The ISRs are edge-triggered by the IR sensor anytime during the device operation until the inactivation of the ISR. The activation of ISRs allow the real-time detection of the user's behaviors of removing the sample tube 148 and the cap 147 from the device 102, 103 (see FIG. 73 for Real-Time Detection of User's Behaviors). When the behaviors are detected, the device calls their corresponding event handling tasks (see FIG. 77 for Event Handling Tasks).

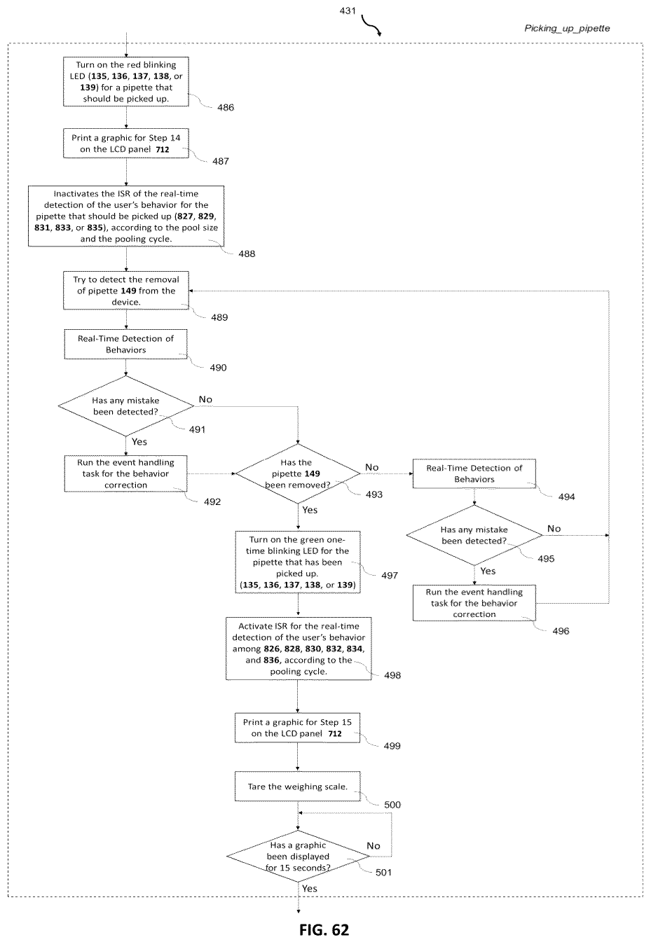

FIG. 62 shows the flow chart for the Picking_up_pipette task 431. For the second task 431—Picking_up_pipette—the device 100 turns on the pipette module receiving slot status LED 135, 136, 137, 138, 139 to blinking red of the corresponding slot of the pipette module 105 as shown in block 486 of FIG. 62. At the same time, the device 100 prints a graphic on the TFT LCD 12 for Step 14 of the sample pooling workflow as shown by block 487 to guide the user to pick up the correct pipette 149. Note that the device 100 inactivates the real-time detection of the user's behavior of removing the pipette from the pipette module 105 (see FIG. 73 for Real-Time Detection of User's Behaviors) as shown by block 488. When the device 100 detects the removal of the pipette 149 by its IR sensor as shown by block 493, the LED turns from red into the green for 1 second and is turned off as shown by block 497. Then the device 100 activates the real-time detection of the user's behavior of real-time detection of putting an object into the slot 118, 119, 120, 121, 122 of the pipette module 105 as shown by block 498.

Next, the device prints a graphic on the TFT LCD 712 for Step 15 of the sample pooling workflow to guide a user to peel off the plastic bag of the individually wrapped pipette as shown by block 499. The graphic is to be printed for 15 seconds as shown by block 501, if the waste-bin module 152 is not available. If the waste-bin module 152 is available, the device waits until the user disposes of the peeled-off plastic bag package into the disposal container 162. When the device detects the disposal action from the user, the LEDs 153, 154, 155, 156, 157, 158, 159, 160, 161 will illuminate the green color for 1 second and are turned off. At the same time, the device tries to tare the weighing scale of the pooling module 107 to measure the pooled sample volume in Step 16. When the taring is done and 15 seconds is up (or the disposal action is detected), the device 100 goes to the next task.

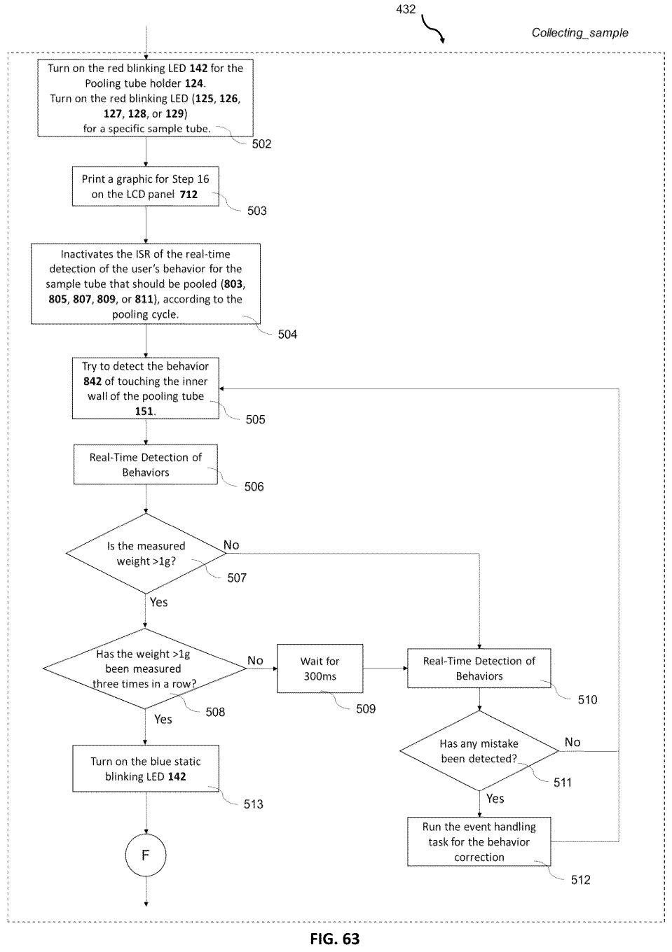

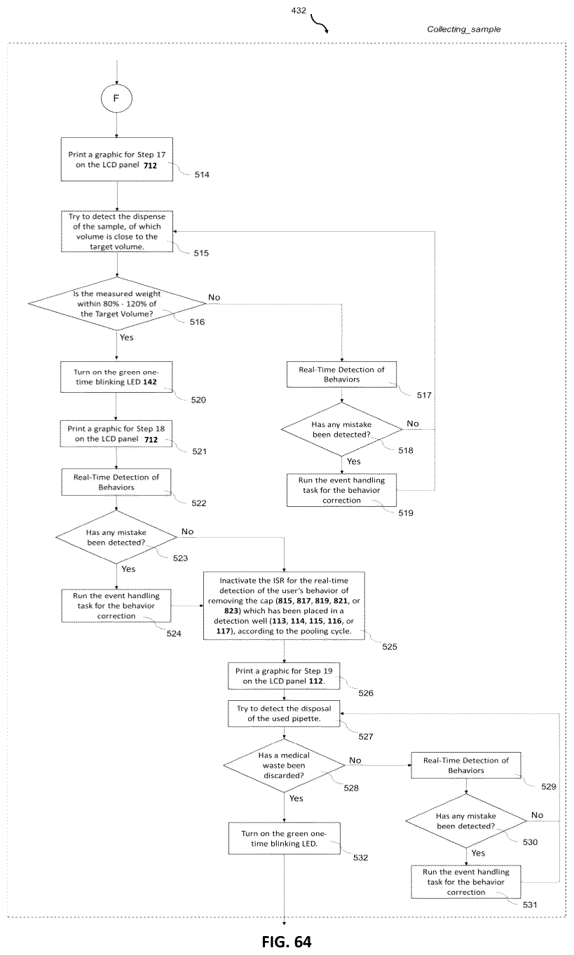

FIG. 63 and FIG. 64 show portions of a flow chart for the Collecting_sample task 432. For the Collecting_sample task 432—the device 100 turns on the pooling tube holder status LED 142 of the pooling module 107 to blinking red and one of the tube module receiving slot status LEDs 125, 126, 127, 128, 129 of the tube module 102 also to blinking red as shown in block 502 of FIG. 63. At the same time, the device 100 prints a graphic on the TFT LCD 712 for Step 16 of the sample pooling workflow to guide the user to aspirate a liquid sample from the sample tube, which should be pooled, and dispense it to the pooling tube as shown by block 503. Then the device 100 waits for the touch of the pipette tip on the pooling tube wall as shown by block 505. The touch is to be detected by measuring the weight of the pooling tube 150 until the weight is, for example, over 1 g, which is much larger than the liquid volume aspirated by the pipette, for some amount of time. The weight detection may be set to other levels depending upon the volume of the pipette.

When the device 100 detects the touching behavior as shown by block 508, the device 100 prints a graphic on the TFT LCD 712 for Step 17 of the sample pooling workflow to guide the user to dispense the liquid sample into the pooling tube as shown by block 514 as shown in FIG. 64. In this step, the device 100 waits for the dispense of the liquid sample as shown by block 515. The dispense is to be detected by measuring the weight of the pooling tube 151 until the weight is within 80%-120% of the target volume for some amount of time as shown by block 516. Depending on the training program, the device on the training mode allows to move forward even if the amount of sample is above 120%.

When the dispensing is performed, the pooling tube holder status LED 142 of the pooling module 107 and the active one of the tube module receiving slot status LEDs 125, 126, 127, 128, 129 of the tube module 102 turn from red into green for 1 second and then are turned off. Then, the device 100 prints a graphic on the TFT LCD 712 for Step 18 of the sample pooling workflow to show that the pooling for a particular sample has been performed successfully as shown by block 521. Note that the real-time detection of removing the tube cap 150 from the cap module 103 is inactivated to allow the user to remove the cap from the device without false alarming after the dispense as shown by block 525.

Then, the device 100 prints a graphic on the TFT LCD 712 for Step 19 of the sample pooling workflow to guide the user to discard the used pipette 149 to the biohazard waste bin as shown by block 526. The graphic for the disposal of the pipette 149 is to be shown for 5 seconds.

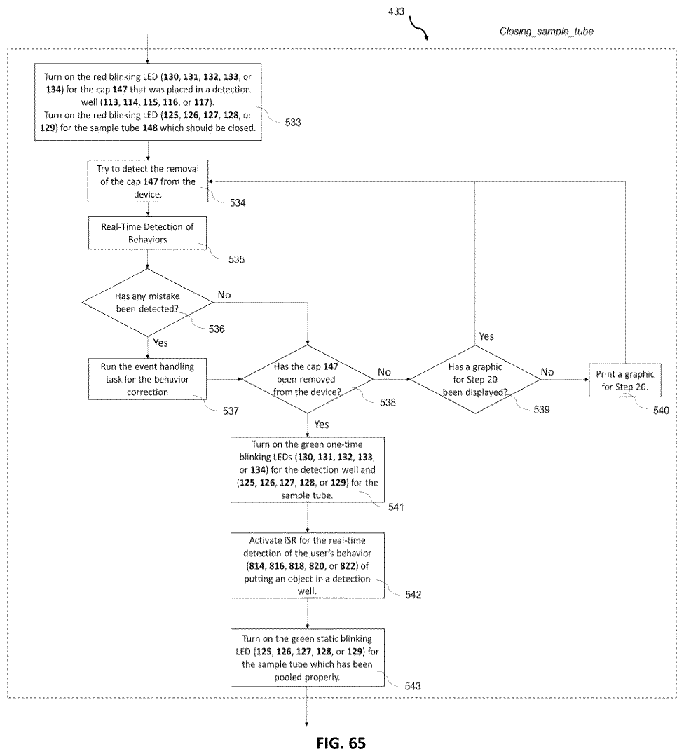

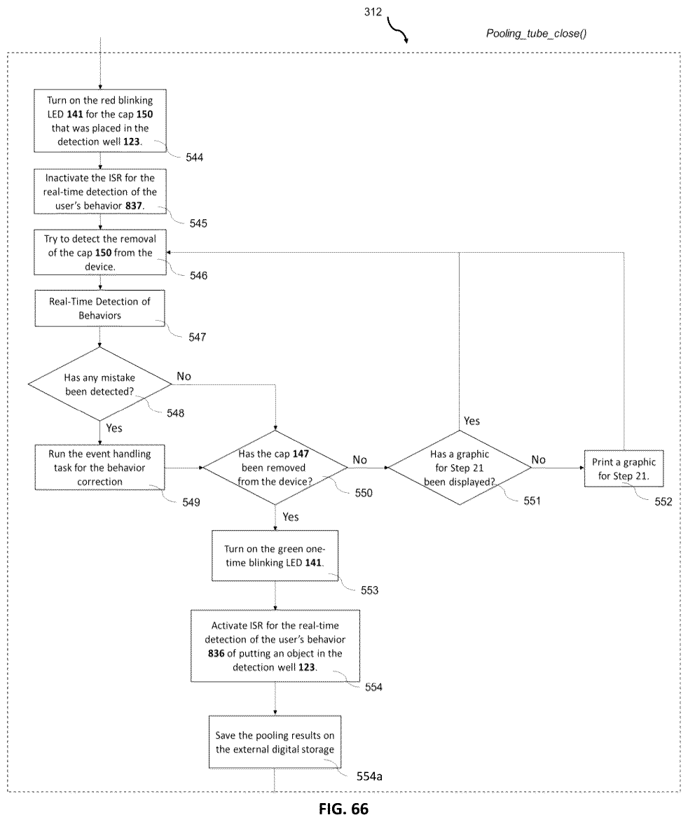

FIG. 65 shows the flow chart for the Closing_sample_tube task 433. For the Closing_sample_tube task 433—the device 100 turns on one of the tube module receiving slot status LEDs 125, 126, 127, 128, 129 to blinking red corresponding to a slot 108, 109, 110, 111, 112 of the tube module 102 as shown in block 533 of FIG. 65. If the user has not closed the tube yet as shown by block 538, the device prints a graphic on the TFT LCD 12 for Step 20 of the sample pooling workflow to guide the user to close the pooled sample tube as shown by block 540. Then the device 100 waits for the removal of the cap 147 from the detection well 113, 114, 115, 116, 117 of the cap module 103 as shown by block 534. When the cap 147 is removed from the device as shown by block 538, the tube module receiving slot status LED 125, 126, 127, 128, 129 turns from blinking red into the green for 1 second and then is turned off as shown by block 541. Then, the device 100 activates the interrupt routine service (ISR) for the IR sensor for the cap 147 of the pooled sample tube 148 as shown by block 542.

The ISR is edge-triggered by the IR sensor anytime during the device operation until the inactivation of the ISR. The activation of ISR allows the real-time detection of the user's behavior of putting any object into the cap detection well 113, 114, 115, 116, 117 of the cap module 103 (see FIG. 73 for Real-Time Detection of User's Behaviors). When the behavior is detected, the device 100 calls its corresponding event handling task (see FIG. 77 for Event Handling Tasks).

Lastly, the device 100 turns on the tube module receiving slot status LED 125, 126, 127, 128, 129 to static green for the pooled sample to let the user know that the sample tube 148 has been already pooled as shown by 543. The pooling cycle is to be performed repeatedly until all sample tubes 148 are pooled successfully, according to the pool size.

The Pooling_tube_close( ) function 312 is to detect whether the user closes the pooling tube 150 properly. FIG. 66 shows the flow chart for the Pooling_tube_close( ) function 312.

The device 100 turns on the pooling cap detection well status LED 141 for the pooling cap 150 to blinking red and waits for the removal of the pooling cap 150 as shown in block 544 of FIG. 66. If the user has not closed the pooling tube 151, the device prints a graphic on the TFT LCD 712 for Step 21 of the sample pooling workflow to guide the user to pick up the pooling cap from the cap detection well and close the pooling tube as shown by block 552. Note that the ISR for real-time detection of the user's behavior of removing the pooling cap is inactivated at this step as shown by block 545. When the pooling cap 150 is removed from the device successfully as shown by block 550, the pooling cap detection well status LED 141 turns from red into green for 1 second and then is turned off as shown by block 553. Then the device 100 activates the ISR for the real-time detection of the user's behavior of putting an object into the pooling cap detection well 123 (see FIG. 73 for Real-Time Detection of User's Behaviors) as shown by block 554. When the behavior is detected, the device calls its corresponding event handling task (see FIG. 77 for Event Handling Tasks).

Finally, the device saves the pooling result on the portable digital storage unit, including an SD card and USB as shown by block 554a. The pooling result includes, but is not limited to, the following details: each volume of pooled samples; and ratios of volumes between pooled samples. As a result, when the sample pooling is finished, the digital storage unit includes, but is not limited to, the following data:
(1) Date, time and day,
(2) The target volume,
(3) Pool size,
(4) Barcode of tune tray,
(5) Unique indicator of tube tray,
(6) Barcodes of sample collection tubes,
(7) Barcode of pooling tube.
(8) Each volume (or weight) of pooled samples, and
(9) Ratios of volumes (or weight) between pooled samples

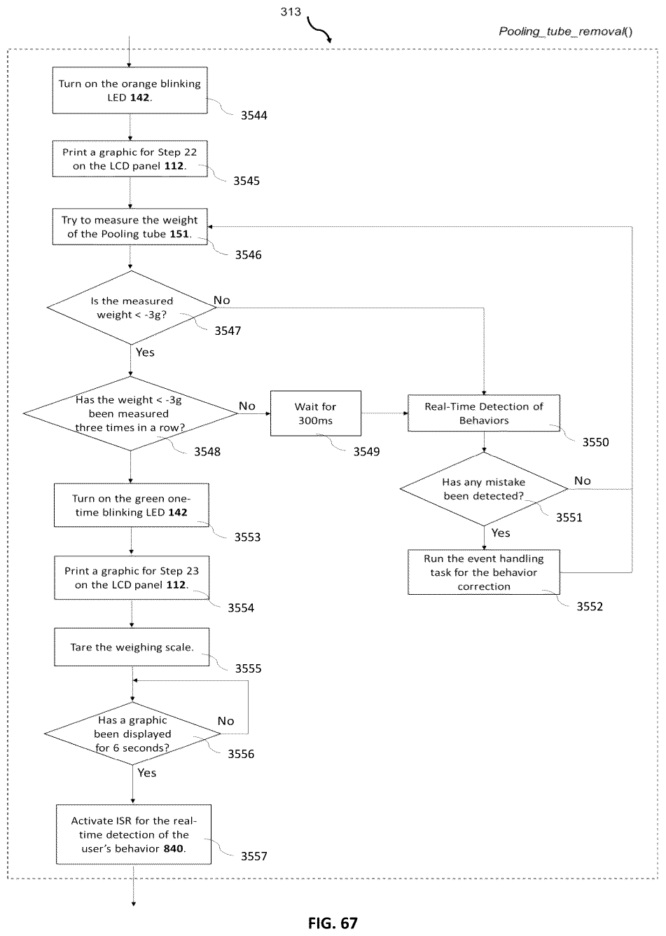

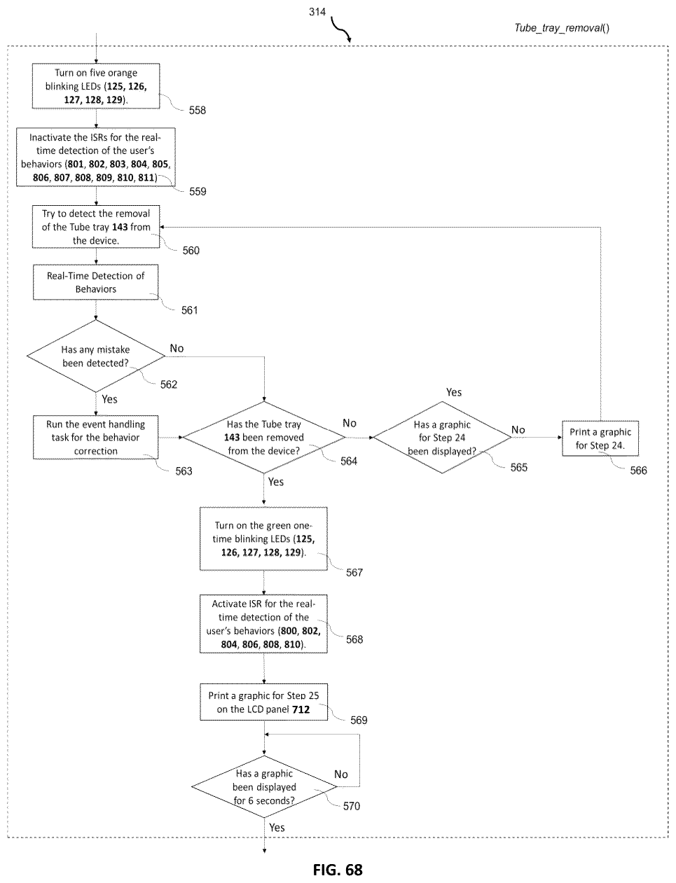

The Pooling_tube_removal( ) function 313 is to detect whether the user removes the pooling tube 151 from the pooling tube holder 124 of the pooling module 107 properly. FIG. 67 shows the flow chart for the Pooling_tube_removal( ) function 313.

The device 100 turns on the pooling tube holder status LED 142 for the pooling tube holder 124 to blinking orange as shown in block 3544 of FIG. 67. The device prints a graphic on the TFT LCD 712 for Step 22 of the sample pooling workflow to guide the user to take the pooling tube 151 from the pooling tube holder 124 as shown by block 3545. Note that the device 100 inactivates the real-time detection of the user's behavior of removing the pooling tube 151 from the pooling module 107. Since the weighing scale has been tared to the weight of the pooling tube 151, the device 100 detects the removal of the pooling tube 151 by measuring the negative weight of, for example, <−3 g. The detection weight may be set to other levels depending upon the weight of the pooling tube. When the pooling tube 151 is removed from the device 100 successfully as shown by block 3548, the pooling tube holder status LED 142 turns green for 1 second and then is turned off as shown by block 3553.

After the removal, the device 100 prints a graphic on the TFT LCD 712 for Step 23 of the sample pooling workflow to guide the user to keep and store the pooling tube for molecular diagnostic testing as shown by block 3554. The graphic is to be shown for 6 seconds as shown by block 3556. At the same time, the device 100 tries to tare the weighing scale of the pooling module 107 in order to detect the presence of unknown objects on the weighing scale correctly as shown by block 3555. After taring, the device 100 activates the real-time detection of the user's behavior of putting an object into the pooling tube holder 124 of the pooling module 107 (see FIG. 73 for Real-Time Detection of User's Behaviors) as shown by block 3557. When the behavior is detected, the device calls its corresponding event handler task (see FIG. 77 for Event Handling Tasks).

The Tube_tray_removal( ) function 314 is to detect whether the user removes the tube tray 143 with pooled sample collection tubes 148 from the tube module 102 properly. FIG. 68 shows the flow chart for the Tube_tray_removal( ) function 314.

The device 100 turns on all of the tube module receiving slot status LEDs 125, 126, 127, 128, 129 of the tube module 102 to blinking orange as shown by block 558 of FIG. 68. If the user has not removed the tube tray 143 from the device 100 yet as shown by block 564, the device prints a graphic on the TFT LCD 712 for Step 24 of the sample pooling workflow to guide the user to take the tube tray 143 with pooled sample tubes 148 from the tube module 102 as shown by block 566. Note that the device 100 inactivates the real-time detection of the user's behaviors of removing the tube tray 143 and/or sample collection tubes 148 from the tube module 102 and putting an object into an empty slot 108, 109, 110, 111, 112 of the tube module 102 as shown by block 559. When the tube tray 143 with sample tubes 148 has been removed from the device properly as shown by block 564, the tube module receiving slot status LEDs 125, 126, 127, 128, 129 turn green for 1 second and are then turned off as shown by block 567. The device 100 activates the real-time detection of the user's behaviors of putting an object into any slot 108, 109, 110, 111, 112 of the tube module 102 (see FIG. 73 for Real-Time Detection of User's Behaviors) as shown by block 568. When one or more of the behaviors are detected, the device calls their corresponding tasks (see FIG. 77 for Event Handling Tasks).

After the removal, the device 100 prints a graphic on the TFT LCD 712 for Step 25 of the sample pooling workflow to guide the user to store the sample tubes for confirmatory testing, which is to be performed when the pooling tube is positive as shown by block 569. The graphic is to be shown for 6 seconds as shown by block 570.

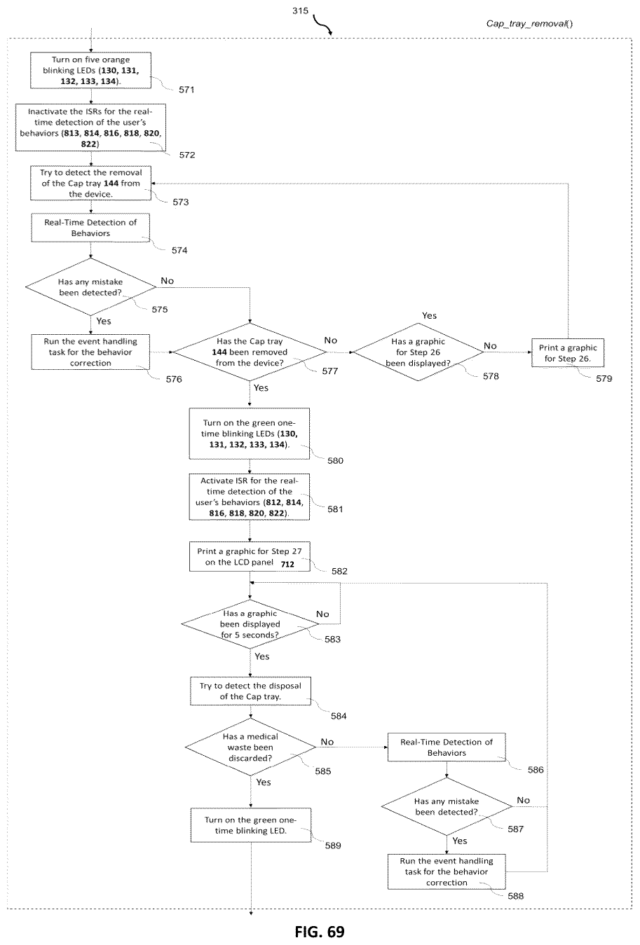

The Cap_tray_removal( ) function 315 is to detect whether the user removes the cap tray 144 from the cap module 103 properly. FIG. 69 shows the flow chart for the Cap_tray_removal( ) function 315.

The device turns on all of the cap module detection well status LEDs 130, 131, 132, 133, 134 of the cap module 103 to blinking orange as shown by block 571 of FIG. 69. If the user has not removed the cap tray 144 from the device yet as shown by block 577, the device 100 prints a graphic on the TFT LCD 712 for Step 26 of the sample pooling workflow to guide the user to remove the cap tray 144 from the cap module 103 as shown by block 579. Note that the device 100 inactivates the real-time detection of the user's behaviors of removing the cap tray 144 from the device 100 and putting any object into the cap module 103 as shown by block 572. When the cap tray 144 has been removed from the device properly 100 as shown by block 577, the cap module detection well status LEDs 130, 131, 132, 133, 134 turn green for 1 second and then are turned off as shown by block 580. The device 100 activates the real-time detection of the user's behaviors of putting an object into any slot of the cap module 103 (see FIG. 73 for Real-Time Detection of User's Behaviors) as shown by block 581. When one or more of the behaviors are detected, the device calls their corresponding event handling tasks (see FIG. 77 for Event Handling Tasks).

After the removal, the device 100 prints a graphic on the TFT LCD 712 for Step 27 of the sample pooling workflow to guide the user to dispose of the cap tray 144 into the biohazard waste bin as shown by block 582.

The device 100 may have an optional waste bin module 152 configured to detect the disposal of items. If the waste bin module 152 is present and the waste bin is not used, a graphic is to be shown for 5 seconds as shown by block 583. If the waste bin module 152 is used, the device activates the ISR for the waste bin module 152 to detect the user's behavior of the disposal in real-time and waits for the disposal of the cap tray 144. At the same time, the device turns on red blinking LEDs 153, 154, 155, 156, 157, 158, 159, 160, 161 for the waste bin module 152. When the disposal is detected, the red LEDs turn into the green LEDs for 1 second and are turned off as shown by block 589. The device inactivates the ISR for the real-time detection of the user's behavior of the disposal.

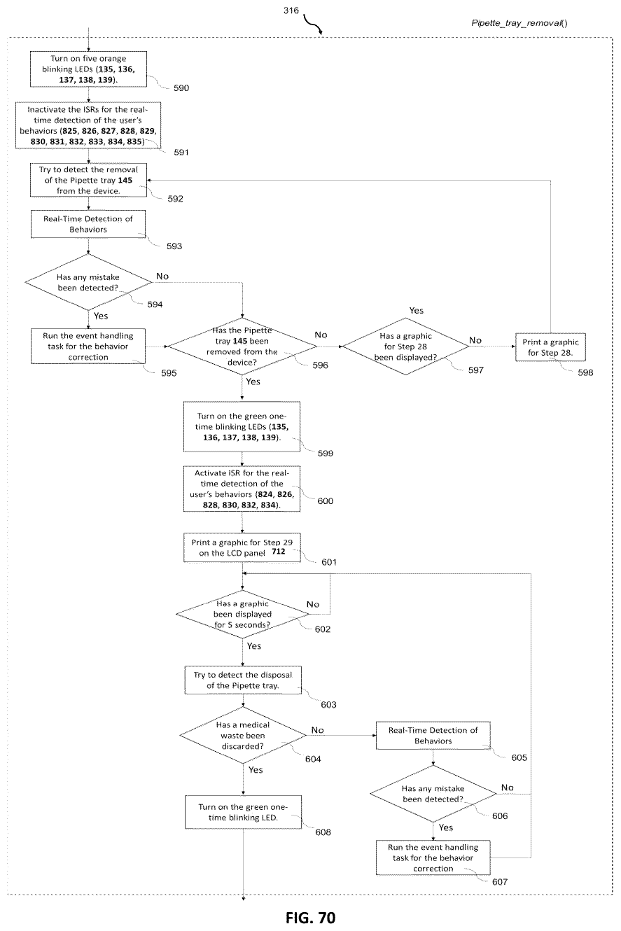

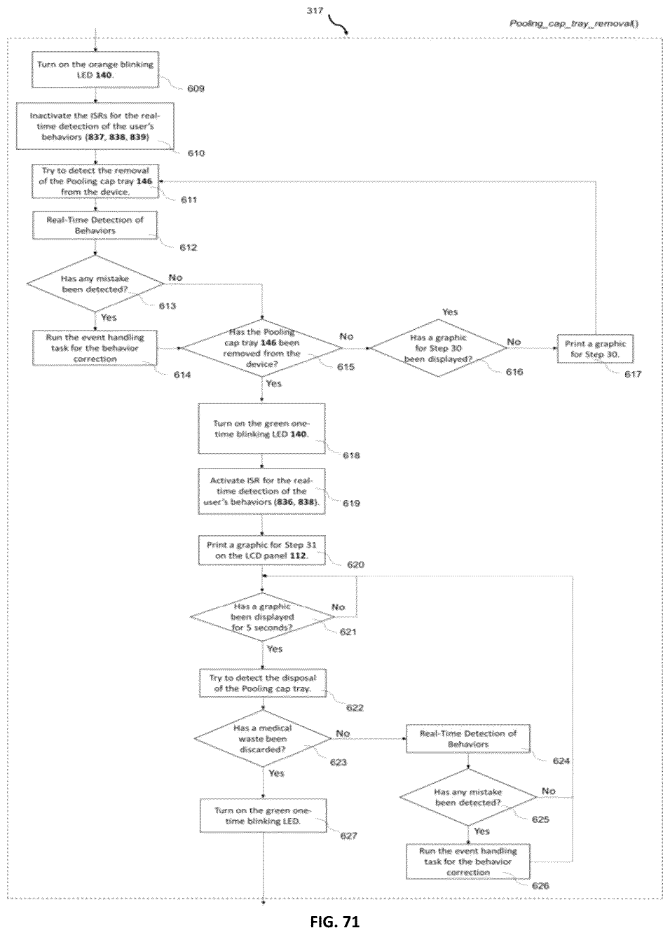

The Pipette_tray_removal( ) function 316 is to detect whether the user removes the pipette tray 145 from the pipette module 105 properly. FIG. 70 shows the flow chart for the Pipette_tray_removal( ) function 316.

The device 100 turns on all pipette module receiving slot status LED 135, 136, 137, 138, 139 of the pipette module 105 to blinking orange as shown by block 590 of FIG. 70. If the user has not removed the pipette tray 145 from the device yet as shown by block 596, the device prints a graphic on the TFT LCD 712 for Step 28 of the sample pooling workflow to guide the user to remove the pipette tray 145 from the pipette module 105 as shown by block 598. Note that the device 100 inactivates the real-time detection of the user's behaviors of removing the pipette tray 145 from the device 100 and putting an object into any slot of the pipette module 105 as shown by block 591. When the pipette tray 145 has been removed from the device properly as shown by block 596, the pipette module receiving slot status LED 135, 136, 137, 138, 139 turn green for 1 second and then are turned off as shown by block 599. The device activates the real-time detection of the user's behaviors of putting any object into any slot 118, 119, 120, 121, 122 of the pipette module 105 (see FIG. 73 for Real-Time Detection of User's Behaviors) as shown by block 600. When one or more of the behaviors are detected, the device calls their corresponding tasks (see FIG. 77 for Event Handling Tasks).

After the removal, the device 100 prints a graphic on the TFT LCD 712 for Step 29 of the sample pooling workflow to guide the user to dispose of the pipette tray 145 into the biohazard waste bin as shown by block 601. If the waste bin module 152 is present and the waste bin is not used, the graphic is to be shown for 5 seconds as shown by block 602. If the waste bin module 152 is used, the device activates the ISR for the waste bin module 152 to detect the user's behavior of the disposal in real-time and waits for the disposal of the pipette tray 145. At the same time, the device turns on red blinking LEDs 153, 154, 155, 156, 157, 158, 159, 160, 161 for the waste bin module. When the disposal is detected, the red LEDs turn into green LEDs for 1 second and are turned off as shown by block 608. The device inactivates the ISR for the real-time detection of the user's behavior of the disposal.

The Pooling_cap_tray_removal( ) function 317 is to detect whether the user removes the pooling cap tray 146 from the pooling module 107 properly. FIG. 71 shows the flow chart for the Pooling_cap_tray_removal( ) function 317.

The device 100 turns on the pooling cap detection well status LED 141 for the pooling cap detection well 123 of the pooling module 107 to blinking orange as shown by block 609 of FIG. 71. If the user has not removed the pooling cap tray 146 from the device 100 yet as shown by block 615, the device 100 prints a graphic on the TFT LCD 712 for Step 30 of the sample pooling workflow to guide the user to remove the pooling cap tray 146 from the pooling module 107 as shown by block 617. Note that the device 100 inactivates the real-time detection of the user's behaviors of removing the pooling cap tray 146 from the pooling module 107 and putting an object into the empty detection well 123 of the pooling module 107 as shown by block 610. When the pooling cap tray 146 has been removed from the device 100 properly as shown by block 615, the pooling cap detection well status LED 141 turns green for 1 second and then is turned off as shown by block 618. The device 100 activates the real-time detection of the user's behaviors of putting an object into an empty detection well 123 of the pooling module 107 (see FIG. 73 for Real-Time Detection of User's Behaviors) as shown by block 619. When one or more of the behaviors are detected, the device calls their corresponding event handling tasks (see FIG. 77 for Event Handling Tasks).

After the removal, the device 100 prints a graphic on the TFT LCD 712 for Step 31 of the sample pooling workflow to guide the user to dispose of the Pooling cap tray into the biohazard waste bin as shown by 620. If the waste bin module 152 is present and the waste bin is not used, the graphic is to be shown for 5 seconds as shown by block 621. If the waste bin module 152 is used, the device 100 activates the ISR for the waste bin module 152 to detect the user's behavior of the disposal in real-time and waits for the disposal of the pooling cap tray 146. At the same time, the device 100 turns on the red blinking LEDs 153, 154, 155, 156, 157, 158, 159, 160, 161 of the waste bin module 152. When the disposal is detected, the red LEDs turn into the green LEDs for 1 second and are turned off as shown by block 627. The device inactivates the ISR for the real-time detection of the user's behavior of the disposal.

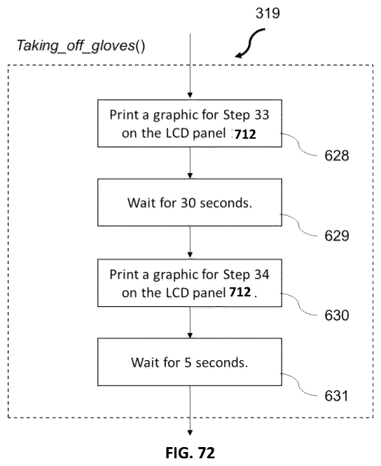

The Taking_off_gloves( ) function 319 is to guide the user to take off gloves and discard them into the biohazard waste bin properly. FIG. 72 shows the flow chart for the Taking_off_gloves( ) function 319.

The device 100 prints a graphic on the TFT LCD 712 for Step 33 of the sample pooling workflow for 30 seconds as shown by blocks 628, 629 of FIG. 72. Lastly, the device prints a graphic on the TFT LCD 712 for Step 34 of the sample pooling workflow for 5 seconds as shown by blocks 630, 631.

The device can detect 44 different kinds of a user's behaviors (or motions) in real-time during the operation. The user's behaviors are detected by IR sensors or the weighing scale controlled by real-time detection functional blocks, each of which can be implemented independently.

FIG. 73 shows real-time detection functional blocks for the 44 different behaviors. In FIG. 73, each block describes the type of behavior and its related object. For example, the Behavior 1 block 800 is the functional block of the real-time detection for the user's behavior of putting the tube tray 143 into the tube module 102. The Behavior 2 block 801 is the functional block of real-time detection for the user's behavior of removing the tube tray 143 from the tube module 102. The Behavior 43 block 842 is the functional block of the real-time detection for the user's behavior of touching the inner wall of the pooling tube 151 by using a pipette tip. The Behavior 44 block 843 is the functional block of the real-time detection for the user's behavior of disposing of an opaque object, including the cap tray 144, the pipette tray 145, the pooling cap tray 146, disposable pipettes 149, and plastic packages for individual pipettes, into the biohazard waste bin.

Among them, Behaviors 1-40 are detected by IR sensors, which are embedded in the tube module 102, the cap module 103, the pipette module 105, and the pooling module 107. Behaviors 41-43 are detected by the variation of the weight. Behavior 44 is detected by small object detection equipment with LEDs, IR sensors, or lasers.

When one of the Behaviors except for Behaviors 43-44 is detected through their corresponding Behavior blocks, the device activates a flag for its corresponding event handling task and calls the task. See FIG. 77 for Event Handling Tasks.

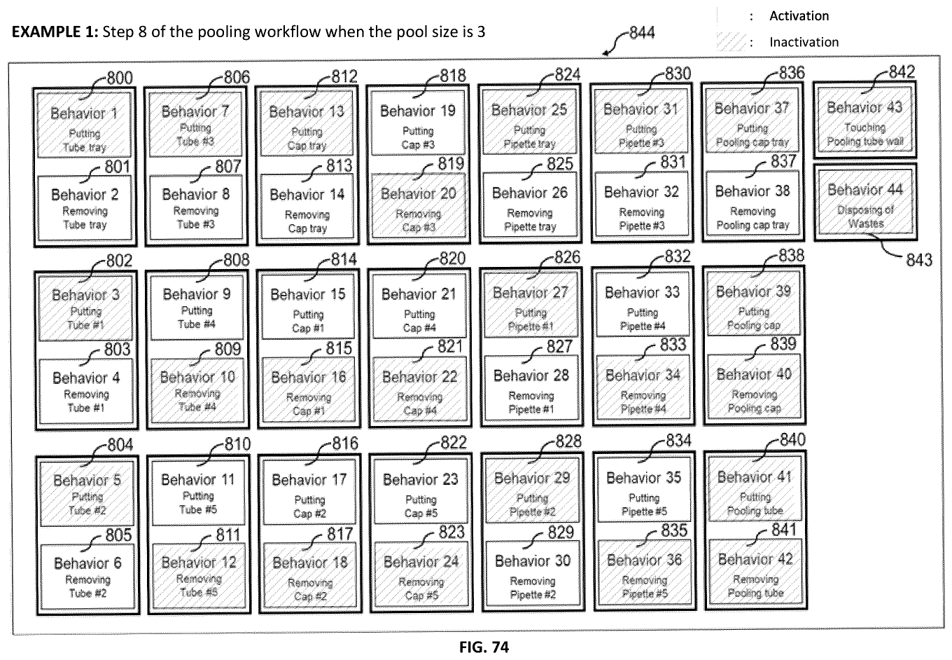

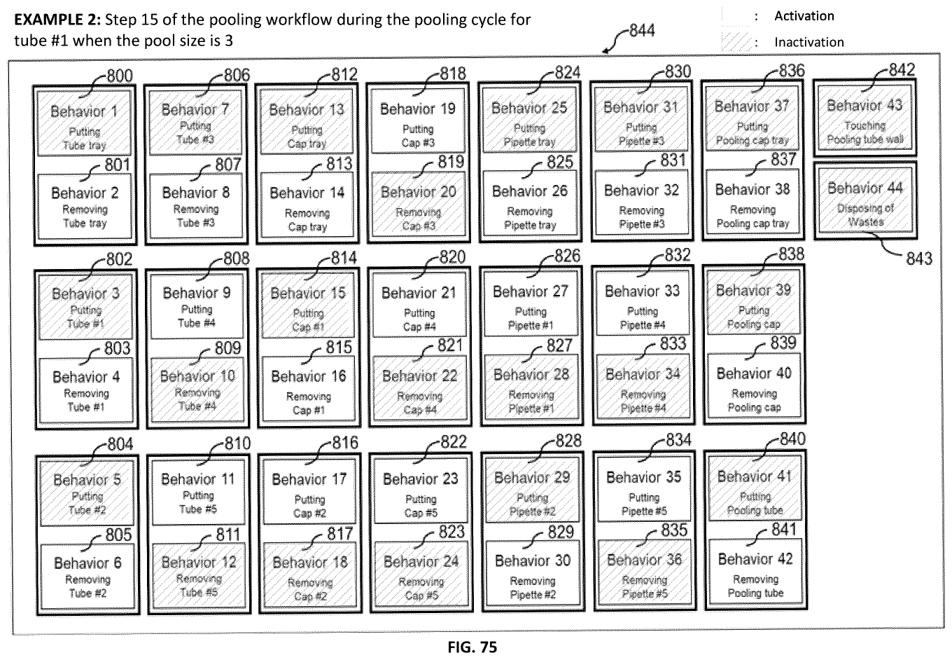

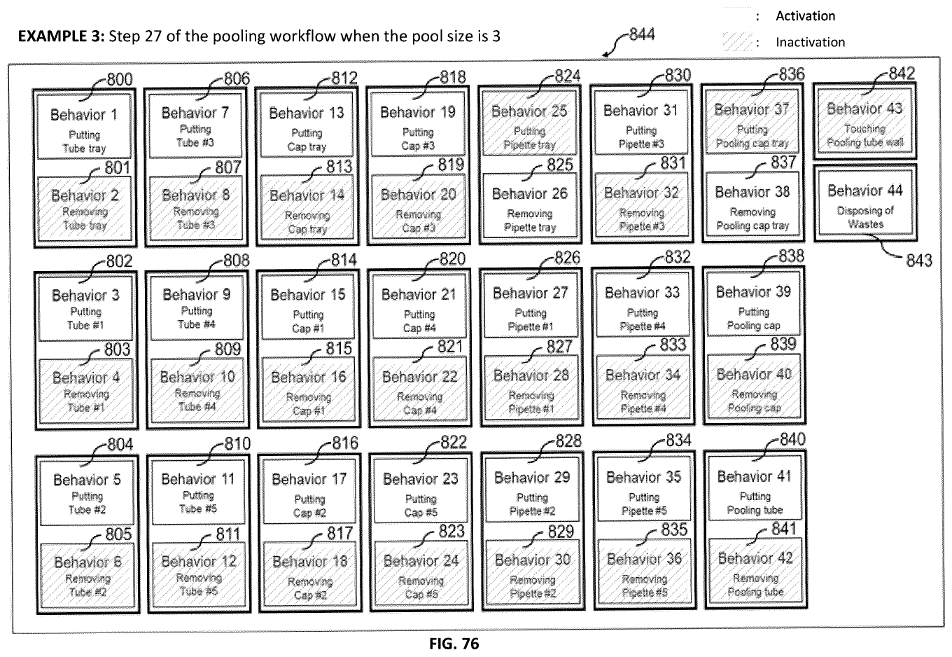

Each real-time detection functional block is activated or inactivated depending on the pooling workflow. The following FIGS. 74, 75, and 76 show examples of activation and inactivation of the Behavior blocks according to the pooling workflow. FIG. 74 shows an example (Example 1) of Step 8 of the pooling workflow when the pool size is 3. FIG. 75 shows an example (Example 2) of Step 15 of the pooling workflow during the pooling cycle for tube #1 when the pool size is 3. FIG. 76 shows an example (Example 3) of Step 27 of the pooling workflow when the pool size is 3.

In this disclosure, ‘event’ is defined as a behavior (or a motion) detected by one of the real-time detection Behavior blocks. The event is considered the user's mistake that should be avoided according to the pooling workflow. Event handling tasks are tasks that are implemented to correct the user's mistakes when corresponding events occur. FIG. 77 shows a block diagram of the event handling tasks.

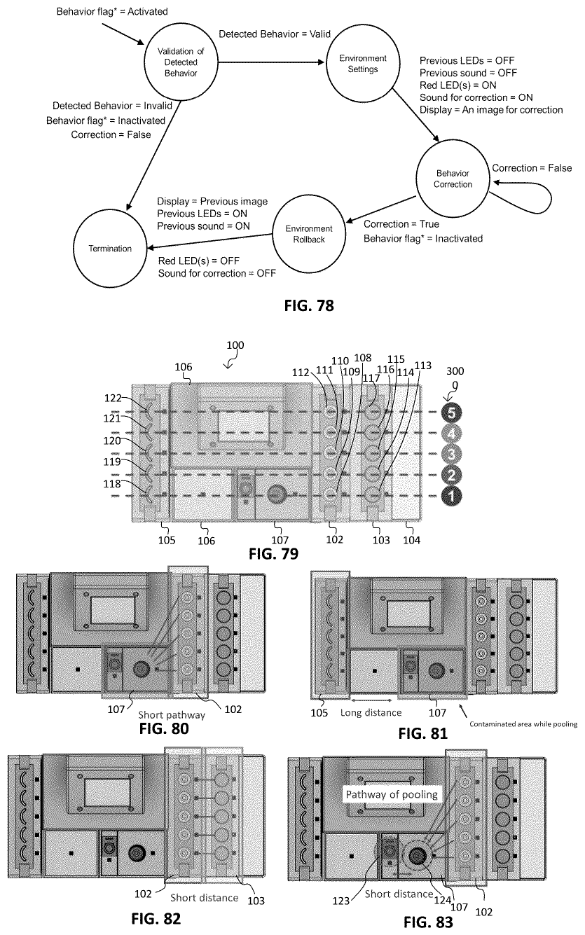

FIG. 78 shows a state machine for the control of each event handling task. The ‘Behavior flag’ shown in FIG. 77 indicates a flag (or a trigger) that has been activated when a real-time detection Behavior block is implemented. When the flag is implemented, the device 100 validates the detected behavior and determines whether the device 100 should go for the Behavior correction loop or not. If the detected behavior is invalid, meaning that the behavior detection is an error, the device 100 inactivates the Behavior flag and terminates the event handling task. If the detected behavior is valid, the device sets the parameters of the device's interactive system environment and runs the loop for the behavior correction. When the mistake is corrected, the device rollbacks the environmental settings and terminates the event handling tasks.

The instructional point-of-care device for sample handling described above has many advantageous features. Although the device described above shows a configuration for a pool size of up to five sample tubes, the modular nature of the device supports other configurations that are scalable to a pool side of 10 or greater. The disposable trays 143, 144, 145, 146 described above are configured to be easy-to-grab trays that protect the device from clinical liquid samples. However, other configurations of the device may be configured to be operated without the trays. The device described above may be configured to have a relatively compact size, with the device having a size of 18.3 inches, roughly equivalent to the size of a large laptop. In that configuration, the device may have a length of 420 mm, approximately equal to the width of an average human shoulder, a width of 200 mm, and a height of 85 mm. The display module 101 may be configured to have a tiltable display to support the use of the device by both disabled and abled users.

The point-of-care device may be configured for an optimized layout for the diagnostic waveform to prevent cross-contamination and minimize biosafety risks. FIG. 79 shows a left-to-right workflow across the modules 101, 102, 103, 104, 105, 106, 107 when the device 100 is used. FIG. 78 also shows the alignment of the pipette slots 118, 119, 120, 121, 122, the tube slots 109, 109, 110, 111, 112, and the cap detection wells 113, 114, 115, 116 designated by slot numbers 3000. This alignment assists the user in ensuring that the proper item is retrieved and placed in each location.

The modules in the device are also laid out to emphasize and reinforce the operations that must be performed for the sample pooling tasks, while also preventing cross-contamination and minimizing biosafety risks. FIG. 80 shows the design of the device where the tube module 103 and pooling module 107 are close to each other. This short pathway of pooling between the pooling module 107 and the tube module 102 helps minimize the possibility of dropping samples. FIG. 81 shows the design of the device where the pipette module 105 and the pooling module 107 are far from each other. This distance allows the pipette 149 unwrapping and handling to be clear from the sample tubes 148 and pooling tube 151. FIG. 82 shows the design of the device where the tube module 102 and cap module 103 are close to each other. This short distance helps ensure that the sample tube caps 147 are properly handled. FIG. 83 shows the design of the device where the pooling cap detection well 123 is located outside the pathway of pooling. This location of the detection well 123 is very close to the pooling tube holder 124 but is located outside the pathway of pooling. Thus, this location helps ensure that the pooling tube cap 150 is not contaminated with a sample from a sample tube 148.

Operational features of the instructional point-of-care device for sampling handling include: real-time detection of presence and absence of objects: sample collection tubes 148, collection caps 147, transfer pipettes 149, the pooling tube 151, and the pooling cap 150; real-time measurement of weight of every pooled liquid sample and calculation of its volume; an interactive system using instructional language-agnostic graphics, indicative colored LED lights, and alarm sounds; an automatic barcode scanning for sample collection tubes 148 and their tray 143; traceable records of sample pooling performance; accessible records of individual sample tubes and pooling results saved on a digital storage unit (SD card etc.); individual sample tracking system from the positive pooled sample via barcodes saved on a digital storage unit (SD card, USB, etc.); various operational modes for specific purposes; customizable operational functions; and, a user management system.

The real-time detection is accomplished by IR sensors inside each module that detect presence and absence in real-time. The real-time detection enables the ability to catch up to 50 or more different kinds of errors. The real-time detection can detect various wrong situations caused by human mistakes during the pooling process.

The real-time measurement of weight is accomplished by the weighing scale that measures the weight of the collected liquid sample in real-time. This weighing scale ensures accurate sampling pooling by using the weight measurements.

The interactive system provides feedback to let the user fix the errors and do the pooling successfully. The system enables adaptive feedback that changes the timing of cues (i.e., visual/audible indications) based on the pace and accuracy with which the user is performing individual steps.

The device has robust records generation and tracking capabilities. The traceable records of sample pooling performance allow a user to check whether the pooling has been performed correctly. The accessible records can allow the device to provide records that will be compatible with an Electronic Medical Records (EMR) system. The device can also be connected to a Laboratory Information Management System (LIMS) via a cellular network, ethernet connection, Bluetooth or other network technologies. The individual sample tracking system provides the user with the ability to easily find individual sample collection tubes and their tray to do confirmatory tests when a pooling tube is tested as positive.

The various operational modes of the device include: the training mode to train a user on how to perform sample handling procedures; an expert mode to help a user to accelerate the sample handling procedures, and an entertainment mode to engage a user to learn the sample handing procedures comfortably. The operational modes and functions of the device can be customized by activating or inactivating each module of the device to enable more than 32 combinations of operational configuration of the device.

The user management system of the device can provide the capability for users to register on the system and keep track of their records through their access cards or identification QR codes. The user management system may also allow administrators to manage the users' records, access the history of sample pooling performances, and customize the device operation for optimization to their workflows in their lab.

Key features of the device include: assistance of improvement in performance of handling skills, which will be measured by volume-transfer accuracy, volume-transfer variability, process time, and handling error rates; acquisition of improved handling skills for better performance of sample pooling which can be measured by higher accuracy, lower variability, and lower error rates, as learning and training effects; adaptation of improved handling skills for better performance of sample pooling, which can be measured by higher accuracy, lower variability, and lower error rates, without assistance from the device; and quality control of handling skill performance for a trained user.

Other key features of the device include: affordability with a cost target estimated at $300 or less; robustness due to the layout of the modules within the device, and reliability due to the limited number and simplicity of the components used in the device. The device may also be configured to be compatible with the upstream and downstream process of existing low-throughput diagnostic devices, such as the Cepheid GeneXpert Xpress CoV-2/RSV/Flu plus device. The device may also be configured to access a cloud data storage system for sample pooling records such as the EMR system and/or LIMS discussed previously. The device provides the ability to be monitored, secured, and only accessed by administrators. Table I below shows an example list of technical specifications for the instructional point-of-care device for sample handling. Other embodiments may have different specifications.

TABLE I

| Modules | Tube | Detection | 1 mL or 3 mL viral transport medium | |
|---|---|---|---|---|
| | | | tubes (not provided) | |
| | | # of IR sensors | 5 (tubes) + 1 (tray) | |
| | | # of LEDs | 5 RGB sockets | |
| | | Tube tray | Storage, barcode attached, provided | |
| | Cap | Detection | Ø 16-20 mm caps | |
| | | # of IR sensors | 5 (caps) + 1 (tray) | |
| | | # of LEDs | 5 RGB sockets | |
| | | Cap tray | Disposable, no barcode, provided | |
| | Pipette | Detection | Individually wrapped exact volume | |
| | | | transfer pipettes (provided) | |
| | | # of IR sensors | 5 (pipettes) + 1 (tray) | |
| | | # of LEDs | 5 RGB sockets | |
| | | Pipette tray | Disposable, no barcode, provided | |
| | Single | # of scanner | 1 (pooling tube) | |
| | Barcode | Barcode type | 1D/2D codes | |
| | | # of LEDs | 1 RGB socket | |
| | Multi- | # of scanners | 5 (tubes) + 1 (tube tray) | |
| | barcode | Barcode type | 1D/2D codes | |
| | Pooling | Allowable tube | Ø 12-16 mm empty pooling tube | |
| | | type | (provided, barcode included) | |
| | | Weight | Weight 1 μg-50 g (1 μg resolution) | |
| | | Measurement | | |
| | | Detection | Ø 12-16 mm caps | |
| | | # of IR sensors | 1 (cap) + 1 (tray) | |
| | | # of LEDs | 1 (tube) + 1 (cap) RGB sockets | |
| | | Pooling cap tray | Disposable, no barcode, provided | |
| | Display | Size | 3.2 | inch |

| | | |
|---|---|---|
| | Resolution | 320 × 240, TFT LCD |
| | Touch panel | Resistive touch |
| | Graphics | 24-bit BMP, no language used |
| Waste-bin | Detection | any object, including wastes generated |
| | | during the process |
| | Weight | Weight 10 μg-500 g (10 μg resolution) |
| | Measurement | |
| | # of PIR sensors | 4 or more |
| | Dimension | Customizable |

| Power | Input voltage | 7-12 | V |
|---|---|---|---|
| Microcontroller | Atmel ARM Cortex M3 | AT91SAM3X8E 84 MHz | |
| | ATMega4809 | ATMega4809 20 MHz | |
| Pool size | | 2-5 (can be extended up to 11) | |
| Storage | SD card | Yes (up to 32 GB) | |
| | USB port | Can be added | |
| Dimensions | Width | 200 mm (without waste-bin module) | |
| | Length | 420 | mm |
| | Height | 85 | mm |

The instructional point-of-care devices and methods for sample handling disclosed herein are relevant to applications and handling of samples in various fields including, but not limited to: medical technology, healthcare, biology, biotechnology, chemistry, chemical engineering, mechanical engineering, electrical engineering, pharmacy, agriculture, food science and production, plant science, and the environment. The devices and methods may be configured to specifically support applications and handling in those fields.

The handling procedures of the disclosed interactive devices and methods may be hands-on processes related to (but not limited to) mixing, stirring, swirling, vortexing, centrifuging, agitating, collecting, combining, merging, synthesizing, blending, injecting, infusing, dripping, loading, metering, sampling, pipetting, aspirating, releasing, dispensing, aliquoting, seeding, inoculating, spreading, dividing, extracting, shuffling, flaming, streaking, staining, smearing, incubating, storing, lifting, pushing, pulling, and carrying. The handling procedures of the disclosed interactive devices may be performed with substances in solid, liquid, and gas, and/or any of those mixtures. Substances may include water, alcohol(s), nutrient media, transport media, buffer(s), oil(s), powder(s), gel(s), and/or volatile gas(es).

The handling procedures of the disclosed interactive devices and methods may be performed with samples related to but not limited to biological samples, chemical substances, medical/clinical, and/or environmental specimens. Samples may be biological samples of any type or mixture of substances containing biological materials related to (but not limited to) cells, tissues, proteins, bacteria, fungi, archaea, protists, germs, parasites, viruses, nucleic acids, bacteriophages, vesicles, lipids, water, oil, blood, plasma, serum, saliva, urine, sputum, semen, sperm, synovial fluids, mucus, cerebrospinal fluids, peritoneal fluids, ascites, sweat, vaginal fluids, amniotic fluids, tears, blisters, cyst fluids, feces, wax, hair, and/or inorganic ions such as sodium ions and potassium ions. Samples may also be chemical substances of any type or mixture of substances, such as those dissolving a solute, causing a chemical reaction, or accelerating a chemical reaction. Samples may also be medical samples of any type or mixture related to (but not be limited to) pills, drugs, powders, tablets, capsules, implants, fluids, creams, topical medications, tinctures, oral washes, and/or suppositories. Samples may also be environmental specimens of any type or mixture that are taken from environments, related to (but not limited to) water, soil, air, minerals, sediments, gases, oil, plants, vegetation, organic matter, livestock, wild animals, insects, and foods.

The handling procedures of the disclosed interactive devices and methods may also be performed with contrived, artificial, and/or synthetic samples related to but not limited to biological samples, medical/clinical, and/or environmental specimens. The contrived, artificial, and/or synthetic samples may have similar physical properties to biological, medical/clinical, and/or environmental samples. The contrived, artificial, and/or synthetic samples may or may not contain chemical and/or biological substances harmful to humans.

The handling procedures of the disclosed interactive devices and methods may be performed with sample-transfer elements, which can carry a sample related to (but not limited to) pipettes, aspirators, dispensers, droppers, samplers, collectors, inoculating tools, cottons, swabs, syringes, forceps, tweezers, scalpels, needles, scoops, spatulas dippers, sponges, cups, and papers and variations of those sample-transfer elements. For example, pipette variations may include (but are not limited to) disposable pipettes, single or multi-channel precise pipettes, serological pipettes, volumetric pipettes, and fixed-volume pipettes.

The handling procedures of the disclosed interactive devices and methods may be performed with sample-holding elements including but not limited to tubes, containers, vials, plates, trays, bags, cups, boxes, pouches, and pads and variations of those sample-holding elements. More particularly, sample-holding elements may include (but are not limited to) transport-media tubes, blood tubes, vacuum tubes, testing vials or tubes, cylinders, parasitology vials, collection devices, ostomy bags, microcontainer tubes, microhematocrit tubes, fecal collection containers, swab collection containers, saliva collection tubes, urine collection containers, concentrators, microcentrifuge tubes, collection containers, specimen containers, tissue culture containers, cell culture gel plates, formalin containers, fixative containers, sample bags, slotted tubes, gas cans, barrels, and absorbent pouches.

The disclosed interactives devices and methods may assist in sample processing through performing automated tasks during or in between user handling procedures. The automated tasks may include (but are not limited to) vortexing, mixing, shaking, filtering, counting, heating, cooling, incubating, rotating, agitating, amplifying, illuminating, precipitating, and/or pelleting.

The handling procedures of the disclosed interactive devices and methods may be performed before putting on personal protective equipment (PPE), which includes but is not limited to laboratory coats, aprons, scrubs, coveralls, sleeves, safety glasses, splash goggles, face shields, facemasks, chemically resistant gloves, footwear, and respirators. Personal protective equipment may not be required for the use of the disclosed interactive devices depending upon the nature of the material being sampled.

The handling procedures of the disclosed interactive devices and methods may include (but are not limited to) sample pooling and gravimetric sample preparation in medical technology, biology, biotechnology, bioengineering, and related fields. The disclosed interactive devices and methods may support molecular diagnostics of which handling procedures may include (but are not limited to) sample collection, sample dilution, inactivation of one or more components, nucleic-acid extraction, lysing, binding, extracting, washing, eluting, filtering, concentrating, amplifying and/or detecting. The disclosed interactive devices and methods may support antigen and/or antibody testing of which handling procedures may include (but are not limited to) sample collection, sample pooling, sample mixing, sample dilution, nucleic-acid extraction, sample incubation, concentration, and/or detection. The disclosed interactive devices and methods may support blood diagnostics of which handling procedures may include (but are not limited to) smearing, fixation, staining, condensing, filtering, blood culture, collection, extraction, sampling, and centrifugation. The disclosed interactive devices and methods may support microbiological examination of which handling procedures may include (but are not limited to) bacterial cell culture, smearing, incubation, flaming, collection, extraction, sampling, sample drawing, dispensing, centrifugation, incubation, streaking, staining, fixation, and disc diffusion procedures. The disclosed interactive devices and methods may support medical diagnostics of which handling procedures may include (but are not limited to) smearing, cell/tissue culture, inoculation, flaming, collection, extraction, sampling, aspiration, sample drawing, dispensing, centrifugation, incubation, streaking, staining, fine needle aspiration (FNA), thawing, aliquoting, and disc diffusion procedures.

The disclosed interactive devices and methods may support handling procedures related to pharmacy, synthetic chemistry, and related fields, and may include (but are not limited to) pharmaceutical medication-dispensing procedures, pharmaceutical pill counting, compounding procedures, and antibody-drug conjugates (ADC) manufacturing procedures. In some procedures, sample making elements are used for preparing samples and may include but are not limited to mortar, pestle, and spatulas. For the medication dispensing procedures, “dispense” or “dispensing” means, according to the Pharmacy Practice Act, the interpretation and evaluation of a prescription, the selection, manipulation or compounding of the medicine, the labelling and supply of the medicine in an appropriate container according to the Medicines Act and the provision of information and instructions to ensure the safe- and effective-use the medicine by the patient. For the compounding procedures, “compounding” means, according to the U.S. Pharmacopeia Convention, the preparation, mixing, assembling, altering, packaging, and labeling of a drug, drug-delivery device, or device in accordance with a licensed practitioner's prescription, medication order, or initiative based on the practitioner/patient/pharmacist/compounder relationship in the course of professional practice.

The disclosed interactive devices and methods may be configured to support handling procedures related to chemistry, chemical engineering, chemical processing, and related fields, and may include (but are not limited to) chemical extractions and purifications, bead-based DNA extractions and/or other forms of DNA extractions. The disclosed interactive devices and methods may assist users in performing sample processing tasks requiring fixed volumes of reagents to be added to one or more samples. The reagents may include samples related to biological samples and/or chemical substances but not limited to water, guanidinium thiocyanate, ethanol, isopropanol, aptamer or antibody containing reagents, and/or other reagents used to wash, precipitate, and/or elute biological agents in a sample. The reagents disclosed herein may be added repeatedly to one or more samples. Distinct reagents may be added serially to one or more samples. The disclosed interactive devices and methods may assist in sample processing through performing automated tasks such as vortexing, mixing, and/or pelleting magnetic particles in a sample being processed. The disclosed interactive devices and methods may assist users in chemical extractions. More particularly, the disclosed interactive devices and methods may assist users in applying polar and/or non-polar solutions to a sample to separate target molecules from the bulk solution. The disclosed interactive devices and methods may mix and/or change the temperature of the sample. The disclosed interactive devices and methods may also guide a user to remove the correct phase (organic, inorganic, or other) of a solution.

The disclosed interactive devices and methods may be configured to support handling procedures for electrical engineering, and related fields, and may include (but are not limited to) printed circuit board (PCB) processing procedures. The disclosed interactive devices and methods may be configured to monitor the addition or removal of passive and active components to a printed circuit board using one or more sensing elements. Components to be added or removed may include but are not limited to resistors, capacitors, inductors, amplifiers, crystals, diodes, jumpers, connectors, ferrite beads, antennas, buttons, regulators, microchips, additional integrated circuits, microprocessors, and/or additional printed circuit boards. The sensing elements used to track PCB processing may include, but are not limited to, weighing scale, an infrared sensor, an ultrasonic sensor, a photoelectric sensor, a photodetector, a camera configured for image recognition and processing, a gas sensor, a polarizer, a stereoscope, a thermometer, a thermistor, a capacitive sensor, a resistive sensor, a strain sensor, and a piezoelectric sensor, configured to ensure a user will achieve the desired result from at least one specific task during the handling procedures.

The interactive devices and methods disclosed herein may use at least one perceptible indication element, which may be related to visual indications, audible indications, and/or tactile indications, or a combination of indications. The visual indications may be performed by visual indicators including but not limited to images, photos, pictures, videos, animations, holograms, lights, markers, symbols, emojis, texts, beams, meters, and colors and/or patterns and/or repetitions of any indicator through visual indicator generators. Visual indicator generators may be provided for generating the visual indicators. The visual indicator generators may include, but are not limited to, display panels, screens, light-emitting diodes (LEDs), organic light-emitting diodes (OLEDs), gauges, projectors, light bulbs, lamps, lasers, piezoelectricity, hologram generators, and stickers. The audible indications may be performed by audible indicators including but not limited to videos, alarms, music, melody, tones, beep sounds, pulses, vibrations, human or animal vocalizations, regular or irregular signals, and/or patterns and/or repetitions of any indicator through audible indicator generators. Audible indicator generators may be provided for generating the audible indicators. The audible indicator generators may include but not be limited to sound generators, such as speakers and buzzers, pulse generators, waveform generators, and vibration generators. The tactile indications may be performed by sensible indicators including but not limited to vibrations, hot/warm/cold temperatures, smoothness or roughness, softness or hardness, finger alphabets and/or braille, static electricity, electrical shock, pop-up features, and intruded/extruded structures, and/or patterns and/or repetitions of any indicator through the touchable indicator generators. Sensible indicator generators may be provided for generating the sensible indicators. The sensible indicator generators may include but are not limited to diaphragms, heaters or coolers, motors, hinges, electrodes, springs, and extruders, and vibration generators.

The interactive devices and methods disclosed herein may perform at least one type of detection, which may be related to object detection, movement detection, behavior detection, and/or signal change detection, or a combination of detection types. Object detection may be provided for detecting at least one object. The detectable objects may include but are not limited to transparent objects, translucent objects, opaque objects, solid objects, aqueous objects, and gaseous objects. Movement detection may be provided for detecting at least one movement of detectable objects. The detectable movements may include but are not limited to the falling of an object, the rotating of an object, the dragging of an object, the streaking of an object, the swing of an object, the vibrating of an object, the mixing of an object.

Behavior detection may be provided for detecting at least one behavior of the user(s) of the devices. The detectable behavior may relate to handling procedures, and more particularly, to hands-on processes of handling procedures, including but not be limited to mixing, stirring, swirling, vortexing, centrifuging, collecting, combining, merging, synthesizing, blending, injecting, dripping, loading, metering, sampling, pipetting, aspirating, releasing, dispensing, aliquoting, seeding, inoculating, spreading, dividing, extracting, shuffling, flaming, streaking, staining, smearing, incubating, storing, lifting, pushing, pulling, carrying, inserting, removing, touching, disposing, opening, closing, scanning, and tilting. The interactive devices and methods may also detect the presence and/or location of the user's body, including the user's hands and/or eyes. The hands/eyes tracking detection associated with perceptible indications may be provided for helping users keep attention to the handling procedures and preventing them making mistakes while the handling procedures. The hands/eyes tracking detection may be performed by cameras.

Signal-change detection may be provided for detecting changes of at least one signal. The detectable changes of signals may be originated from various parameters related to but not limited to volume, weight, color, light intensity, turbidity, optical density, temperature, gas, height, distance, capacitance, resistance, force, and pressure.

The detections may be performed by detecting elements. The detecting elements may include object sensing elements, movement sensing elements, behavior sensing elements, and/or signal change sensing elements, or a combination of any elements. Object detection may be performed by at least one object-sensing element that is configured to detect at least one detectable object inside a device. The object sensing elements may include but are not limited to infrared sensors, photoelectric sensors, capacitive sensors, inductive sensors, magnetic proximity sensors, ultrasonic sensors, weighing scales, and cameras. Object detection may be associated with at least one perceptible indication, such as at least one visual indication, at least one audible indication, at least one tactile indication, and/or combinations of indications, configured to provide a user with the information of the presence or absence of at least one detectable object.

Movement detection may be performed by at least one movement-sensing element configured to detect at least one detectable movement. Movement-sensing elements may include but are not limited to infrared sensors, piezoelectric sensors, accelerometers, gyroscopes, magnetometers, strain sensors, ultrasonic sensors, capacitive circuitry and/or sensors (such as mutual capacitance and/or self-capacitance) and cameras. The movement detection may be associated with at least one perceptible indication, such as at least one visual indication, at least one audible indication, and/or at least one tactile indication, and/or combination of indications, configured to provide a user with the information of at least one detectable movement of at least one detectable object or the user.

Signal-change detection may be performed by at least one signal-change-detection element configured to detect changes of at least one signal from various parameters. The signal-change-sensing elements may include object-sensing elements and movement-sensing elements. The signal change detection may be associated with at least one perceptible indication, such as at least one visual indication, at least one audible indication, and at least one combination of at least one visual indication and at least one audible indication, configured to catch a user's mistake or unknown errors during one or more steps of the handling procedures. The signal change detection elements may also relate to various measurement tools including but not be limited to a weighing scale, an infrared sensor, an ultrasonic sensor, a photoelectric sensor, a photodetector, a camera configured for image recognition and processing, a turbidity meter, a gas sensor, a polarizer, a stereoscope, a fluorometer, a thermometer, a thermistor, a capacitive sensor, a resistive sensor, a strain sensor, and a piezoelectric sensor, configured to ensure a user to get a reliable result from at least one specific task during the handling procedures.

Some of the detection types may be performed in real-time, provided for real-time interaction with users to give the user information about the user's handling performance and help the user to improve the performance of the handling process. The real-time detections may be the detection of interest within 1 second. The real-time detections may be performed by at least one detecting element and/or at least one perceptible indication in order to provide real-time perceptible feedback to users. The real-time detections may be performed by at least one combination of detecting elements in real-time. The real-time detections may be performed by at least one combination of perceptible indications in real-time. The real-time detections may also be performed by at least one combination of at least one detecting element and at least one perceptible indication in real-time.

The devices and methods disclosed herein relate to interactive devices and methods, and more particularly, to devices and methods for entertaining and educating novice users through performing handling procedures using contrived, artificial, and/or synthesized samples. The invention is relevant to entertainments and educational applications in various fields including but not limited to medical technology, healthcare, biology, biotechnology, chemistry, chemical engineering, mechanical engineering, electrical engineering, pharmacy, agriculture, food science and production, plant science, and the environment.

As disclosed herein, the devices and methods may be configured to provide interactive devices and methods for entertainment. The devices and methods may be configured to provide instructional operations for helping users learn and follow handling procedures. The devices and methods may also be configured to provide operations for encouraging creativity of thinking and experimentation by users. The interactive devices and methods for entertainment disclosed herein may provide reinforcement cues to encourage user engagement and correct operation. The cues may include perceptible indications including but not limited to visual and/or auditory feedback, such as displaying colors and/or animations, and/or playing cheers, chimes, and/or verbal messages, related to the user's behavior at a given step and designed to encourage correct device operation and discourage incorrect device operation. The interactive devices and methods for entertainment disclosed herein may keep a score of the user's performance. The score may be stored for more than one user and users' scores can be presented alongside each other in a scoreboard. The interactive devices and methods for entertainment disclosed herein may use at least one perceptible indication element. The interactive devices and methods for entertainment may be performed by detecting elements. The interactive devices and methods for entertainment may assist in educating the user to the background, motivations, and/or technical aspects of a sample handling procedure. Education may be achieved using perceptible indications including one or more audio and/or visual relays programmed to provide educational information regarding sample handling.

As disclosed herein, the interactive devices and methods also relate to the field of interactive medical devices, and more particularly, to methods and devices for guiding users through one or more of the handling procedures. More particularly, the handling procedures of the disclosed interactive medical devices and methods may include but are not limited to sample pooling, gravimetry, and handling procedures in molecular testing, immunoassays, blood testing, microbiological examination, medical diagnosis, proteomics and pharmacy. The interactive medical devices and methods may provide instructional operations for guiding handling procedures. The interactive medical devices and methods may also provide operations for training users, including unskilled users, in sample-handling procedures and workflows related to medical technology. The interactive medical devices and methods may also provide operations for improving the efficiency and/or efficacy of sample-handling procedures and workflows of users, including skilled users.

The interactive medical devices and methods may provide operations for user engagement for handling procedures and workflows. The interactive medical devices and methods may be used as a model for training and user engagement for handling procedures and workflows using contrived, artificial, and/or synthesized samples that do not contain any biological components.

The interactive medical devices and methods may provide at least one combination of the operations. The interactive medical devices and methods may provide at least one operation related to at least one of the sample pooling procedures. For example, some sample pooling procedures may include but not be limited to identification of patients, personal protective equipment (PPE) usage, disinfection, disposal, collection, dilution, aspiration, dispensing, sampling, pooling, and/or squeezing. The interactive medical devices and methods may provide at least one operation related to gravimetric sample preparation. For example, some gravimetric sample preparation procedures may relate to but not be limited to include PPE usage, pH adjustment, precipitation, digestion, filtration, washing, drying, ignition, a weight measurement, and/or calculation. The interactive medical devices and methods may provide at least one operation related to at least one of the sample handling procedures for molecular testing. For example, some sample handling procedures for molecular testing may include but not be limited to are identification of patients, PPE usage, disinfection, disposal, dispensing, cell culture, viral culture, bacterial culture, fungal culture, pathogen culture, sample collection, cell lysis, solid-phase extraction of nucleic acids, elution, amplification, and/or detection. The interactive medical devices and methods may provide at least one operation related to at least one of the sample handling procedures for immunoassays. For example, some sample handling procedures for immunoassays, such as antigen or antibody tests, enzyme-linked immunosorbent assays (ELISAs), and immunochemical and immunohistochemical staining, may include, but are not limited to, identification of patients, PPE usage, disinfection, disposal, sample collection, thawing, cell culture, aliquoting, centrifugation, extraction, sedimentation of whole blood, dispensing, and/or incubation.

The interactive medical devices and methods may assist users in performing immunoassays through perceptible indications supporting correct user steps, including but not limited to timing incubations, applying the correct amount of liquid or solid reagents, and/or ensuring reactions occur in the correct sequence and at the correct temperature. The interactive medical devices and methods may also perform the automated tasks to assist users in immunoassays and sample handling, including but not limited to controlling sample temperature, controlling the amount of light that illuminates a sample (including darkening or illuminating a sample), vortexing, mixing, and/or shaking samples, and pelleting and/or agitating magnetic particles in a sample.

The interactive medical devices and methods may provide at least one operation related to at least one of the sample handling procedures for blood testing. For example, some sample handling procedures for blood testing that the medical devices and methods deal with may include but not be limited to identification of patients, PPE usage, disinfection, disposal, smear, blood culture, collection, sedimentation of whole blood, extraction, sampling, staining, fixation, dispensing, and/or centrifugation.

The interactive medical devices and methods may provide at least one operation related to at least one of the sample handling procedures for microbiological examination. For example, some sample handling procedures for microbiological examination that the medical devices and methods deal with may include but not be limited to identification of patients, PPE usage, disinfection, disposal, smear, bacterial cell culture, fungal culture, viral culture, inoculation, flaming, colony count, collection, extraction, sampling, sample drawing, dispensing, centrifugation, incubation, streaking, staining, and disc diffusion procedures.

The interactive medical devices and methods may provide at least one operation related to at least one of the sample handling procedures in medical diagnostic laboratories. For example, some sample handling procedures of medical diagnosis that the medical devices and methods deal with may include but are not limited to identification of patients, PPE usage, disinfection, disposal, smear, bacterial cell culture, fungal culture, viral culture, tissue culture, inoculation, flaming, collection, extraction, sampling, aspiration, sample drawing, dispensing, centrifugation, incubation, streaking, staining, fine needle aspiration (FNA), thawing, aliquoting, and/or disc diffusion procedures.

The interactive medical devices and methods may provide at least one operation related to at least one of the handling procedures, including medication dispensing procedures and compounding procedures, in pharmacy. For example, some handling procedures in pharmacy that the medical devices and methods deal with may include but not be limited to identification of patients, identification of medications, compounding, mixing, dispensing, dividing, wrapping, and/or disposal.

The interactive medical devices and methods may provide at least one operation for sample pooling of various infectious diseases; various sexually transmitted diseases, various cancer diseases, and miscellaneous diseases. The various infectious diseases include, but are not limited to, SARS-CoV, SARS-CoV-2, Flu, Tuberculosis, Enteroviral Meningitis, Strep A, Respiratory syncytial virus, MRSA, Soil-transmitted helminth infections, Malaria, Hepatitis B, Hepatitis C, West Nile, Ebola, and/or Zika. The various sexually transmitted diseases include, but are not limited to, Trichomonas vaginalis, Chlamydia trachomatis, Neisseria gonorrhea, Group B Streptococcus, hepatitis, herpes simplex virus (HSV), human papillomavirus (HPV), syphilis, and human immunodeficiency virus (HIV). The various cancer diseases include, but are not limited to, prostate cancer, colorectal cancer, ovarian cancer, lung adenocarcinoma, liver cancer, and/or breast cancer. The various miscellaneous diseases include, but are not limited to, cervical friability, pelvic inflammatory disease, cervicitis, urethritis statuses, Creutzfeldt-Jakob disease (CJD), and/or Coxiella burnetii.

The interactive medical devices and methods may provide at least one operation for sample pooling using various liquid samples, various solid samples, and various biological samples The liquid samples for sample pooling may include, but are not limited to, sputum, saliva, blood, serum, plasma, sperm, semen, urine, synovial fluids, cerebrospinal fluids, peritoneal fluids, amniotic fluids, mucus, sweat, transport media, water, buffer, and/or oil. The solid samples for sample pooling may include, but are not limited to, swabs, feces, powders, pharmaceutical medications, proteins, and/or tissues. The various biological samples may include, but are not limited to, those containing proteins, nucleic acids (e.g., DNA and RNA), mammalian cells, bacterial cells, fungal cells, and/or viruses.

The interactive medical devices and methods may provide at least one operation related to at least one of the adaptive sample pooling strategies and/or at least one of the non-adaptive sample pooling strategies. The adaptive pooling strategies include, but are not limited to, the Dorfman pooling algorithm, S-Stage algorithm, binary splitting by Halving algorithm, binary splitting algorithm, multi-stage pooling algorithm, and/or Sterrett pooling algorithm. The non-adaptive sample pooling strategies include, but are not limited to, DNA sudoku, multidimensional pooling, combinatorial tapestry pooling algorithms, and matrix and/or array testing. At least one of the sample pooling strategies may be executed with a different order, including a reverse order. The interactive medical devices and methods may also provide at least one operation related to at least one of the sample pooling types including but not limited to media pooling and/or swab pooling.

The interactive medical devices and methods may provide at least one operation for at least one sample pooling workflow dependent upon the number of sample-holding elements. For example, at least one operation may be performed from a single sample-holding element to multiple sample-holding elements. Alternatively, at least one operation may be performed from multiple sample-holding elements to a single sample-holding element. Further, at least one operation may be performed from multiple sample-holding elements to multiple sample-holding elements.

The interactive medical devices and methods may provide at least one operation for sample pooling for various applications, which may include but not limited to gene sequencing, diagnostics, screening, monitoring, and surveillance programs for humans, livestock, wildlife, birds, and insects. For example, the interactive medical devices and methods for sample pooling may be used for various diagnostic testing in diagnostics including, but not limited to, molecular testing, antigen testing, and antibody testing. As another example, the interactive medical devices and methods for sample pooling may be used for monitoring, including, but not limited to, identification of samples with detectable concentrations of pathogenic agents (e.g., viral load), identification of samples with very high concentrations of pathogenic agents (e.g., viral load), and identification of samples with concentrations of pathogenic agents (e.g., viral load) above a certain concentration of pathogenic agents (e.g., a threshold of viral load).

The interactive medical devices and methods provide one or more operations for sample pooling with different approaches. Approaches for sample pooling may include, but are not limited to, pooling of a single type of sample from two or more different individuals, and pooling of two or more different types of samples from a single individual.

The interactive medical devices may comprise several compartments and may provide users with guidance for the sample handling procedures with various detecting elements and various perceptible indicators. The interactive medical devices may comprise at least one compartment with at least one detecting element and at least one indicator for a perceptible indication. In addition, the compartments of the interactive medical devices may be able to contain sample-holding elements, sample-transfer elements, and detectable objects. The interactive medical devices may also comprise at least one compartment with at least one processor to perform at least one type of detection, including real-time detections. The interactive medical devices may also comprise at least one compartment with at least one digital repository of medical records related to, but not limited to, identification of a patient, identification of a patient's sample, barcode numbers of a patient, barcode numbers of a patient's sample, medical history, sample handling results, and diagnoses. The medical records saved on at least one digital repository of the interactive medical devices may be linked to an electronic medical record (EMR) system. The interactive medical devices may comprise at least one compartment with at least one processor associated with at least one digital repository configured to perform at least one function of reading and writing data such as reading and saving barcodes, reading and saving sample handling results, and reading graphic images.

The interactive medical devices may comprise at least one compartment with at least one processor associated with at least one perceptible indication configured to provide a user perceptible feedback for the user's sample handling performance. The at least one compartment with at least one processor may be associated with at least one visual indicator generator configured to guide a user to follow instructions for a sample handling procedure. The at least one compartment with at least one processor may be associated with at least one light source, including but not limited to LEDs, bulbs, lamps, lasers, and hologram generators, configured to guide a user to correct at least one mistake. At least one compartment with at least one processor may be associated with at least one display panel, including but not limited to LCD displays, LED displays, and electronic-ink displays. The at least one compartment with at least one processor may be associated with at least one audible indicator generator, such as buzzers, speakers, and vibrators, configured to guide a user to correct at least one mistake.

The interactive medical devices may comprise combinations of at least one compartment with at least one processor associated with at least one digital repository and at least one perceptible indication and at least one compartment with at least one detecting element and at least one indicator for a perceptible indication.

The interactive medical devices disclosed herein may provide at least one visual indicator associated with at least one processor to visually convey information to a user. The interactive medical devices may display images, display pictures, display photos, play videos, and/or play animations, describing one or more steps of sample handling procedures. Images may comprise language-agnostic images describing one or more steps of the sample handling procedures. Pictures may comprise language-agnostic pictures describing one or more steps of the sample handling procedures.

The interactive medical devices may display barcodes containing information related to but not limited to medical records, certification, a webpage address, a download link, an operational mode of devices, and a parameter value of devices. The interactive medical devices may display texts describing a measured value of one or more parameters.

The interactive medical devices may illuminate light indicating one or more steps of sample handling procedures. The light illumination may be light with various colors, including but not limited to red, blue, green, pink, orange, white, and yellow, configured to inform the status of one or more steps of sample handling procedures. The light illumination may also comprise blinking of colored light at various intervals, such as 500 milliseconds intervals, one-second intervals, and two-second intervals, configured to inform the status of one or more steps of sample handling procedures.

The interactive medical devices may use symbols or markers describing information related to but not limited to the location of an object, the type of an object, and one or more steps of sample handling procedures. For example, some symbols may indicate the location where sample-transfer elements should be put in. The interactive medical devices may also use meters, gauges, and colors to give users information on the performance of sample handling procedures.

The interactive medical devices may use combinations of visual indicators in order to help users get feedback in various perceptible ways.

The interactive medical devices may provide at least one audible indicator associated with at least one processor to audibly convey information to a user. The interactive medical devices may generate sounds at an audible frequency within the range between 20 Hz and 20 kHz. Constant sounds may be generated to provide for notifying critical issues on the medical devices. One-time beep sounds may be generated to provide for notifying errors or mistakes during the sample handling procedures. Frequent beep sounds may be generated at various intervals, such as 500 milliseconds intervals, one-second intervals, and two-second intervals, to provide for notifying errors or mistakes during the sample handling procedure. Alarming sounds may be generated to provide for notifying errors or mistakes during the sample handling procedures. Music may be generated to provide for engaging users to the sample handling procedures. Melody may be generated to provide for engaging users to the sample handling procedures. Tones may be generated to provide for engaging users to the sample handling procedures. Voices may be generated to provide for engaging users to the sample handling procedures. Combinations of audible indicators may be used in order to help users get feedback in various perceptible ways.

The interactive medical devices may perform the object detection. Some of object sensing elements may detect the detectable objects in real-time to confirm that a user follows instructions for a sample handling procedure. The detectable objects in real-time may include, but are not limited to, various lab supplies or medical supplies including, but not limited to: vials, tubes, pipettes, vial caps, tube caps, swabs, tongue blades, sample collection devices, sampling scoops, needles, inoculation loops, gloves, wipers, masks, Petri dishes, well plates, cell culture flask, paper bags, plastic vinyl packages, microscope glasses, magnets, and disposable plastics. The interactive medical devices may comprise at least one compartment with at least one object sensing element, including real-time object sensing elements, associated with at least one processor and at least one perceptible indicator. The interactive medical devices may use at least one object sensing element associated with: at least one visual indicator to visually display information of real-time detection of objects to a user; with at least one audible indicator to audibly convey information of real-time detection of objects to a user; or at least one visual indicator and at least one audible indicator to audibly convey information of real-time detection of objects to a user.

The interactive medical devices may comprise at least one compartment with at least one real-time object sensing element. The interactive medical devices may have at least one processor configured to perform various types of real-time object detection, including but not limited to, the presence or absence of an object, the movement of an object, changes of an object, and changes of compounds of an object, to encourage a user to feel confident in completing one or more steps of sample handling.

The interactive medical devices may perform movement detection with movement sensing elements. Some of movement sensing elements may detect the detectable movements in real-time to check whether a user follows instructions for a sample handling process or not. The detectable movements in real-time may include various motions including, but not limited to, the falling of an object, the rotating of an object, the dragging of an object, the streaking of an object, the swing of an object, the swing of a human's hand, the vibrating of an object, and the mixing of an object.

The interactive medical devices may comprise at least one compartment with at least one movement sensing element, including real-time movement sensing elements, associated with at least one processor and at least one perceptible indicator. At least one movement sensing element may be associated with at least one visual indicator to visually display information of real-time detection of movements to a user. At least one movement sensing element may be associated with at least one audible indicator to audibly convey information of real-time detection of movements to a user. The at least one movement sensing element may be associated with combinations of at least one visual indicator and at least one audible indicator to audibly convey information of real-time detection of movements to a user.

The interactive medical devices may perform behavior detection with behavior sensing elements. Some of the behavior sensing elements may detect the detectable behaviors in real-time to check whether a user makes at least one mistake during a sample handling process or not. The detectable behaviors in real-time may include various motions including, but not limited to, putting an object into a device, removing an object from a device, touching an object, disposing of an object, opening an object, closing an object, scanning an object, and tilting an object.

The interactive medical devices may comprise at least one compartment with at least one behavior sensing element, including real-time behavior sensing elements, associated with at least one processor and at least one perceptible indicator. The at least one behavior sensing element may be associated with: at least one visual indicator to visually display information of real-time detection of behaviors to a user; at least one audible indicator to audibly convey information of real-time detection of behaviors to a user, or with combinations of at least one visual indicator and at least one audible indicator to audibly convey information of real-time detection of behaviors to a user.

The interactive medical devices may perform measurement for various parameters to measure the performance of a user's specific task at one or more steps of sample handling procedures. Some measurements may be performed in real-time. The parameters may include weight, volume, optical density, turbidity, light intensity, temperature, gas, height, distance, capacitance, resistance, force, and pressure. The measurements, including real-time measurements, may be performed in at least one compartment using various measurement tools, including, but not limited to, a weighing scale, an infrared sensor, an ultrasonic sensor, a photoelectric sensor, a photodetector, a camera configured for image recognition and processing, a turbidity meter, a gas sensor, a polarizer, a stereoscope, a fluorometer, a thermometer, a thermistor, a capacitive sensor, a resistive sensor, a strain sensor, and a piezoelectric sensor, configured to ensure a user gets a reliable measurement for at least one specific task during the sample handling procedures. The measurements, including real-time measurement, may be performed in at least one compartment associated with at least one processor and at least one digital repository to save information to inform a user of a measured value to make sure that a user did at least one specific task successfully. The measurements, including real-time measurements, may be performed with at least one processor, in at least one compartment associated with at least one perceptible indication The interactive medical devices may perform the real-time measurements flexibly, where each measurement can be enabled and disabled at one or more steps of the sample handling procedures.

The interactive medical devices may perform real-time detection of changes to catch a user's mistake or unknown errors during one or more steps of the sample handling procedures. The detectable changes in real-time may include various changes related to, but not limited to, volume, weight, color, light intensity, turbidity, optical density, temperature, gas, height, distance, capacitance, resistance, force, and pressure. The real-time detection of some changes may be performed with o: at least one processor using at least one object sensing element; at least one movement sensing element; or at least one real-time measurement tool, which is associated with at least one perceptible indication. Real-time detection of changes may be performed flexibly, where each detection change can be enabled and disabled at one or more steps of the sample handling procedures.

The interactive medical devices may provide barcode scanning in at least one compartment associated with at least one processor and configured to identify an object, read information, and save the information on at least one digital repository. Barcode scanning may be performed using at least one barcode scanner or at least one CCD camera configured to identify an object or read an input from a user to set a parameter of the device operation. Information encoded in a barcode may relate to identification of an object, and more particularly, for sample pooling procedures, to identifiers and parameters such as: a character indicating a disposable tray; a numeric indicator for a disposable tray; a symbolic indicator indicating a coordinate of a disposable tray; a pool size; a desired pooling volume of each individual sample; and an indicator of sample type. Information encoded in a barcode may also include parameters of operation related to, but not limited to, activation and inactivation of functions, and time setting for one or more steps of the sample handling procedures. Information encoded in a barcode may also include parameters of various operational modes, such as: training mode to train a user how to perform sample handling procedures; expert mode to help a user to accelerate the sample handling procedures; and entertaining mode to engage a user to learn the sample handling procedures comfortably.

The interactive medical devices may have multiple combinations of customized operations and configurations activated by or inactivated by compartments of the devices. The compartments may include, but not be limited, to compartments detecting sample-holding elements, compartments detecting lab/medical supplies, compartments detecting sample-transfer elements, compartments used to identify samples and device materials, compartments measuring various parameters for pooling, compartments detecting lab/medical wastes, and compartments for perceptible indications. The interactive devices may comprise modular platforms. The interactive medical devices may be modular platforms having modular components comprising: modular components detecting sample-transfer elements, modular components detecting sample-holding elements, modular components measuring various parameters for pooling, modular components detecting lab/medical supplies, modular components used to identify samples and device materials, and modular components detecting lab/medical wastes. Each modular component may provide at least one perceptible indication associated with detections and measurement.

The interactive medical devices may be presented as a kit of parts, where various modular components are initially in an un-assembled configuration and are assembled together by the user prior to use of the device. If needed or desired, a subset of the various modular components (not necessarily all of them) can be assembled, depending on the particular use, availability, and/or location of the devices. The interactive medical devices comprising assembled modular components may have multiple combinations of customized operations and configurations by activated or inactivated compartments of the devices.

The interactive medical devices may be configured with an optimized design for user's handling pathways in the biosafety point of view and the cross-contamination point of view. For sample pooling purposes, compartments detecting sample-holding elements may be located next to compartments measuring various parameters for pooling, configured to minimize the possibility of dripping clinical samples while transferring the liquid samples. For sample pooling purposes, compartments detecting sample-transfer elements may be located far from compartments measuring various parameters for pooling, configured to minimize the contamination of clinical samples to the clean transfer pipettes. For sample pooling purposes, compartments detecting sample-holding elements are located next to compartments detecting lab/medical supplies, configured to minimize the possibility of dripping clinical samples while opening the tubes.

The interactive medical devices may interact with remote sensing elements and feedback elements, which may be not necessarily physically attached to the devices, to help guide users to the correct samples to be used in the sample-handling procedures. The remote sensing elements may be provided for tracking samples and/or sample-holding elements stored in storage units. The storage units may include, but are not limited to, refrigerators, deep freezers, cold rooms, incubators, warehouses, boxes, racks, shelves, containers, and drawers. The remote sensing elements may include, but are not limited to, infrared sensors, ultrasonic sensors, accelerometers, gyroscopes, temperature sensors, barcode scanners, cameras, lasers, and/or a combination of these elements. The remote sensing elements may be used to detect users' behaviors when users access at least one storage unit of samples (or sample-holding elements). The detectable behaviors pertaining to the storage of samples (or sample-holding elements) may include, but are not limited to, opening and/or closing doors of a storage unit and sliding of racks and/or drawers. The detection of users' behaviors pertaining to the storage may be performed in real-time. The remote sensing elements may be associated with at least one processor configured to check whether the detected users' behaviors are appropriate in users' handling procedures and to communicate with the interactive devices disclosed herein, EMR, LIMS, and/or a cloud system. The remote sensing elements may be associated with at least one digital repository configured to access information on samples and/or sample-holding elements. The remote sensing elements may be installed inside and/or outside at least one storage unit and may communicate with interactive devices through communication technologies with wired and/or wireless network. The communication technologies may include ethernet, Bluetooth, Wi-Fi, cellular networks, radio-frequency (RF) communications, and/or infrared communication The remote sensing elements may identify information on samples and/or sample-holding elements stored in at least one storage unit. The information may include, but is not limited to, patients, diseases, sample types, stored samples' locations in at least one storage unit, sample volumes, and/or sample weights.

A system may be provided for tracking and managing samples and/or sample-holding elements and giving users feedback on their handling procedures for storage and/or testing. For example, with sample pooling procedures, the user may need to access the correct individual sample-holding elements when a single-pooled sample-holding element is positive. In this case, the system may give the user perceptible feedback about the locations of individual sample-holding elements through blinking red LEDs. The system for tracking and managing samples, or the sample management system, may comprise at least one processor and at least one remote sensing element and/or at least one cloud system as a virtual digital repository for information on samples and/or sample-holding elements. The system for tracking and managing samples may be linked to an EMR system and/or an LIMS. Feedback elements may include perceptible indications including, but not limited, to buzzers, light indicators, LEDs, and OLEDs which interact with sensing elements to provide user feedback regarding correct sample handling, including collecting the correct samples and putting samples back into their correct respective storage locations.

The interactive medical devices may provide a kit to ensure precise handling of samples. The kit may include components needed for handling procedures, such as reagents and protective elements that protect the devices from misuse. The kit may be consumables as medical wastes and the costs associated with dealing with those wastes. The kit may be disposable and the kit may include at least one disposable component configured to avoid contaminations. The kit may be prepackaged and the kit may include at least one prepackaged component configured to make the kit clean and/or sterile. The kit may be recyclable, and the kit may include at least one recyclable component configured to reduce generated wastes. The kit may be biodegradable, and the kit may include at least one component produced by biodegradable materials configured to reduce the contamination to the environment. The kit may be produced with combinations of properties such as disposability, prepackaging, recyclability, and biodegradability. For sample pooling procedures, the kit may contain several components including, but not limited to, sample-holding elements for sample collection, sample-holding elements for sample pooling, prepackaged sample-transfer elements, disposable plastic elements, and visual indicator generators.

The interactive devices may provide methods for operations for receiving input from users. Before performing a handling process, the interactive devices may wait for input from users for better device operation for guiding handling procedures. Ways to receive input from users may include, but are not limited to, scanning barcodes, scanning of graphics, pushing buttons of the devices, sliding switches, and connecting identifiers to the devices. The interactive devices may wait for identifying the operational modes that a user wants. Identifying the operational modes may enable a user to choose the device operation in three different modes: training mode, expert mode, and entertainment mode. For sample pooling procedures, receiving input in various ways may enable users to implement an individual sample tracking system that is provided for finding individual sample-holding elements from a pooled sample holding element. The individual sample tracking system may inform users of the location of individual sample tubes when the devices identify the pooled sample-holding element. For example, when the device reads the barcode of the pooling tube that has been recorded on external digital storage, the device may show the location of its individual sample tubes.

The interactive devices may provide methods for operations using perceptible indications to guide a user to perform handling procedures. The interactive devices may operate visual indications of which a sequence is based on a workflow of handling procedures and the interaction between the devices and users. A sequence of graphics, including instructional images, pictures, and photos, on the display panel may be provided. Language-agnostic graphics may be used to help a user understand easily, without any language, a task that is required in a step of a sample handling procedure. The graphics may be stored in at least one digital repository associated with a processor of devices, configured to visually display information to a user.

The interactive medical devices may operate in the training mode using graphics for several individual steps, including but not limited to, PPE preparation, disinfection, set-up of disposable protective elements, set-up of individual sample-holding elements in a compartment detecting sample-holding elements, identification of individual sample-holding elements, set-up of lab/medical supplies in a compartment detecting the supplies, set-up of sample-transfer elements in the compartment detecting the sample-transfer elements, set-up of a disposable protective element for a pooled sample-holding element, identification of the pooled sample-holding element, set-up of the pooled sample-holding element in a compartment detecting the pooled sample-holding element, opening of the pooled sample-holding element, opening of an individual sample-holding element, pick-up of a sample-transfer element, sampling from the individual sample-holding element, pooling of sample into the pooled sample-holding element, confirmation of the pooling performance, disposal of the used sample-transfer element, closing of the opened individual sample-holding element, closing of the opened pooled sample-holding element, removal of the pooled sample-holding element from the device, storing of the pooled sample-holding element, removal of the individual sample-holding elements and their protective element from the device, storing of the individual sample-holding elements into a storage, removal of disposable protective elements from the device, disposal of the protective elements, disinfection, removal of PPE from the user, and disposal of the used PPE. This training mode may be used in support of a Dorfman sample pooling procedure using liquid samples.

The interactive medical devices may comprise a device that may support a training mode having individual steps in the training mode sequence which may include, but are not limited to: putting on gloves; disinfecting the workplace; setting up a tube tray with sample tubes; scanning barcodes of tubes; setting up a cap tray; setting up a pipette tray with individually wrapped disposable transfer pipettes; setting up a pooling cap tray; scanning the barcode of a pooling tube; opening the pooling tube and putting its cap into a cap detection well of the device; putting the pooling tube into the device; picking up and opening a sample tube; putting the sample tube and its cap into the device; scanning the barcode of the sample tube; picking up a corresponding transfer pipette; peeling off the plastic bag package of the pipette; aspirating the liquid sample from the sample tube; dispensing the liquid sample into the pooling tube; confirming the pooling result; disposing of the used pipette; closing the pooled sample tube; closing the pooling tube; removing the pooling tube from the device; storing the pooling tube for molecular testing; removing the tube tray with sample tubes from the device; storing the tubes for potential confirmatory testing; removing the cap tray from the device; disposing of the cap tray into the biohazard waste bin; removing the pipette tray from the device; disposing of the pipette tray into the biohazard waste bin; removing the pooling cap tray from the device; disposing of the pooling cap tray into the biohazard waste bin; disinfecting the workplace; taking off and disposing of the gloves into the biohazard waste bin; and showing the success mark. This training mode sequence may be used in support of a Dorfman sample pooling procedure using liquid samples. Among graphics for the above steps, the following graphics may be repeated until all sample tubes are pooled successfully: picking up and opening a sample tube; putting the sample tube and its cap into the device; scanning the barcode of the sample tube; picking up a corresponding transfer pipette; peeling off the plastic bag package of the pipette; aspirating the liquid sample from the sample tube; dispensing the liquid sample into the pooling tube; confirming the pooling result; disposing of the used pipette.

The interactive medical devices may operate in an expert mode using graphics for several individual steps including, but not limited to: set-up of disposable protective elements, set-up of individual sample-holding elements in a compartment detecting sample-holding elements, identification of the individual sample-holding elements, set-up of lab/medical supplies in a compartment detecting the supplies, set-up of sample-transfer elements in the compartment detecting the sample-transfer elements, identification of a pooled sample-holding element, set-up of the pooled sample-holding element in the compartment detecting the pooled sample-holding element, opening of the pooled sample-holding element, opening of an individual sample-holding element, pick-up of a sample-transfer element, pooling of sample into the pooled sample-holding element, confirmation of the pooling performance, closing of the opened individual sample-holding element, closing of the opened pooled sample-holding element, removal of the pooled sample-holding element from the device, removal of the individual sample-holding elements and their protective element from the device, removal of disposable protective elements from the device, disposal of the protective elements, disinfection, removal of PPE from the user, and disposal of the used PPE. This expert mode may be used in support of a Dorfman sample pooling procedure. The sequence of graphics may be changed if at least one human error (or a mistake) is detected during the sample handling procedure. A display panel of the device may show a specific graphic that describes the detected suspicious behavior and how to correct the behavior. The display panel of the device may also be configured to not show a specific graphic if its corresponding step has been completed successfully before displaying the specific graphic.

The interactive medical device may comprise a device that supports operation in an expert mode having individual steps in the expert mode sequence that may include, but are not limited to: setting up a tube tray with sample tubes; scanning barcodes of tubes; setting up disposable trays (e.g., a pipette tray, a cap tray, and a pooling cap tray); scanning the barcode of a pooling tube; opening the pooling tube and putting its cap into the cap detection well of the device; opening the sample tube and putting the tube and its cap into the device; picking up a corresponding transfer pipette; pooling the liquid sample from the sample tube into the pooling tube; confirming the pooling result; disposing of the used pipette; closing the pooled sample tube; closing and removing the pooling tube from the device; removing the tube tray with sample tubes from the device; removing all disposable trays (e.g., a pipette tray, a cap tray, and a pooling cap tray) from the device and disposing of them into the biohazard waste bin; disinfecting the workplace; taking off and disposing of the gloves into the biohazard waste bin; showing the success mark. These steps may be used in support of a Dorfman sample pooling procedure using liquid samples. The device may provide graphics for the steps described above, where the following graphics may be repeated until all sample tubes are pooled successfully: opening the sample tube and putting the tube and its cap into the device; picking up a corresponding transfer pipette; pooling the liquid sample from the sample tube into the pooling tube; confirming the pooling result; disposing of the used pipette; closing the pooled sample tube. The sequence of graphics may be changed if at least one human error (or a mistake) is detected during the sample handling procedure. A display panel of the device may show a specific graphic that describes the detected suspicious behavior and how to correct the behavior. The display panel of the device may also be configured to not show a specific graphic if its corresponding step has been completed successfully before displaying the specific graphic.

The interactive devices may play instructional videos on a display panel, where the videos may include: ideal handling features of users for specific steps, and visual or audible reactions from the device to help a user understand the procedure; cases of the device operation when the device detects at least one human error (or a mistake) to help a user be aware of the cases; cases regarding biosafety issues associated with the sample handling procedure and the device operation to help a user aware the cases; for example, a video may show a case when a user drops an opened tube with clinical liquid sample on the workplace; and cases regarding chemical safety issues associated with the sample handling procedure and the device operation to help a user aware the cases; for example, a video may show a case when a user sprays an ethanol liquid on the workplace or a user's body.

The interactive devices may comprise an interactive device that provides a sequence of visual indications based on a workflow of sample handling procedures and interactions between the device and a user. The visual indications may comprise light sources. The light sources may be controlled by internal algorithms based on: the status of a current step and/or detection results by corresponding sensing elements. The light sources may illuminate several different colors, including but not limited to: red color of at least one light source located next to a region of interest where a user should perform a hands-on task, when a step of the handling procedure has not been completed; blue color of at least one light source located next to the region of interest where an object related to a current step exists, when a step of the handling procedure is in progress; pink color of at least one light source located next to a region of interest where an object unrelated to a current step exists, when a step of the handling procedure is in progress; orange color of at least one light source located next to a region of interest where used disposable protective elements exist, when a step to let a user remove the protective elements from the device has not been completed; and, green color of at least one light source located next to a region of interest where the user should perform a hands-on task, when a required task for the step of the handling procedure is completed. The red color may blink at a specific interval, including 500-millisecond intervals, one-second intervals, and two-second intervals, from the beginning of a step of the handling procedure in order to let a user know where the user should perform a hands-on task. The blue color may be static to inform the user that a specific sample is to be pooled but not in the current step. The pink color may blink at a specific interval, including 500-millisecond intervals, one-second intervals, and two-second intervals, while an object(s) is in a specific detection region(s), which should have been empty. The orange color may blink at a specific interval, including 500-millisecond intervals, one-second intervals, and two-second intervals, until a used protective element is removed from the device. The green color may blink only once for a few seconds in order to inform that a hands-on task at the region of interest has been completed. The green color may be static when one of required tasks for a single task has been completed at its corresponding region of interest and the other is in progress. The sequence of the visual indications may be changed if at least one human error (or a mistake) is detected during the handling procedures. All visual indicators may be off and at least one light source may be turned on when a display panel of the device may show a specific graphic that describes a detected suspicious behavior(s). The sequence of the light sources may work based on the handling procedures, if no human error (or a mistake) is detected or if a video showing the handling procedures is displayed.

The interactive devices may comprise an interactive device that provides a sequence of audible indications based on a workflow of sample handling procedures and interactions between the device and a user. The sequence of the audible indications may be provided based on the detection of human errors (or mistakes). The audible indicators may comprise audible indicator generators that make a sound at various intervals, including 500-millisecond intervals, one-second intervals, and two-second intervals, when a human error(s) (or a mistake) is detected by corresponding sensing elements.

The interactive devices may include internal algorithms to control perceptible indications such as visual indicators, audible indicators, or other perceivable indicators. The interactive devices may check whether a requested task has been performed successfully or not before displaying a graphic for a step of a handling procedure. If a user has performed before displaying the graphic for the current step, the device perceives that the current step has been finished and that the graphic for the current step should not be displayed and goes for the next step. If a user has not performed a required task in the current step, the device displays the graphic for the current step.

The interactive devices may include internal algorithms to catch various errors, including human errors, using combinations of multiple different detections, including real-time detections. The interactive devices may detect multiple different errors from external sources, such as vibration by an earthquake or strong winds. For sample pooling procedures, the devices may detect multiple different human behaviors such as putting sample-holding elements into compartments of the devices, touching the sample-holding elements, and disposing of wastes by object sensing elements and measurement tools in real-time. The devices may dynamically activate and inactivate the detection of human behaviors to determine whether the detected behavior is a human error (or a mistake). The devices may implement perceptible indications as outputs when the device detects at least one error, including a human error, as an input. The output is configured to guide a user to correct all detected errors (or mistakes). The device waits for the correction of all errors before resuming the sample handling procedure. The internal algorithm to catch errors, including human errors, may consist of at least one following stages including, but not limited to: real-time detection of activated human behaviors; activation of event handling tasks specific to certain human behaviors; implementation of specific event handling tasks; and screening of false detections and validity test of the detected behavior. The devices may detect human behaviors by catching the changes of signals from object sensing elements and real-time measurement tools. For example, the devices may detect the removal of objects from the device by catching the changes of object detection signals from existence to absence in real-time. When a certain human behavior is detected, the device may activate event handling tasks for the detected behavior which has been considered an error, including a human error and a mistake. The devices may implement activated event handling tasks. The devices may test the validity of the detection of activated human behaviors described above to screen the false detections. For example, once the device detects the removal of objects from the device, the device confirms whether the objects are indeed absent right now or not.

The interactive devices may include internal algorithms to correct various errors, including human errors, using combinations of multiple different detections, including real-time detections. When an interactive device detects at least one error, including a human error and a mistake, the device may pause the existing task assigned for a step of sample handling procedure and start a new task for a correction step of each detected error. The device can remember the existing task; the device can go back to the original task after finishing tasks for corrections of errors, including human errors and mistakes. When the device detects at least one corrected human behavior, the device may double-check the validity of the corrected behavior. For example, when the user puts back a specific tube into the device, then the device detects the existence of object by an object sensing element, and then confirms that the detected object is a tube that the device has looked for. The confirmation tool could be performed by scanning the barcode of tube and checking whether the scanned barcode is the one recorded on the digital repository.

The internal algorithm to correct errors may consist of at least one following stage, including, but not limited to: implementation of perceptible indicators; real-time detection of corrected human behaviors; inactivation of event handling tasks specific to certain behaviors; and re-implementation of original tasks with perceptible indicators. With implementation of perceptible indicators, when an interactive device confirms the detected behavior is an error, including a human error and a mistake, the device turns off all perceptible indicators and implements specific perceptible indicators to guide a user to correct the error(s). For example, the device turns off display, LEDs, and buzzer sounds and then turns on a display with a specific language-agnostic graphic describing the correction of the detected error, one-second interval red blinking LEDs at locations where the error occurred, and one-second interval buzzer sounds. With real-time detection, an interactive device may wait for the correction of errors, including human errors and mistakes, by the user. In some cases, the device checks the validity of the correction by using identifiers, such as barcode scanners, or other measurement tools. For inactivation of event handling tasks, when the detected error is corrected, an interactive device may inactivate the event handling tasks specific to certain human behaviors. For re-implementation of original tasks, an interactive device may roll back all settings and implement the original tasks.

For sample pooling procedures, interactive devices may include internal algorithms to perform a Dorfman pooling strategy as well as other pooling strategies such as matrix pooling strategy. For a Dorfman sample pooling procedure and other pooling strategies, the interactive medical devices may operate in a training mode with internal algorithms that consist of several individual steps, including but not limited to: PPE preparation, disinfection, set-up of disposable protective elements, set-up of individual sample-holding elements in the compartment detecting sample-holding elements, identification of the individual sample-holding elements, set-up of lab/medical supplies in the compartment detecting the supplies, set-up of sample-transfer elements in the compartment detecting the sample-transfer elements, set-up of a disposable protective element for a pooled sample-holding element, identification of the pooled sample-holding element, set-up of the pooled sample-holding element in the compartment detecting the pooled sample-holding element, opening of the pooled sample-holding element, opening of an individual sample-holding element, pick-up of a sample-transfer element, sampling from the individual sample-holding element, pooling of sample into the pooled sample-holding element, confirmation of the pooling performance, disposal of the used sample-transfer element, closing of the opened individual sample-holding element, closing of the opened pooled sample-holding element, removal of the pooled sample-holding element from the device, storing of the pooled sample-holding element, removal of the individual sample-holding elements and their protective element from the device, storing of the individual sample-holding elements into a storage, removal of disposable protective elements from the device, disposal of the protective elements, disinfection, removal of PPE from the user, and disposal of the used PPE.

For a Dorfman sample pooling procedure using liquid samples or other strategies using liquid samples, the interactive medical devices may operate in a training mode with internal algorithms that consist of several individual steps, including but not limited to: putting on gloves; disinfecting the workplace; setting up a tube tray with sample tubes; scanning barcodes of tubes; setting up a cap tray; setting up a pipette tray with individually wrapped disposable transfer pipettes; setting up a pooling cap tray; scanning the barcode of the pooling tube; opening the pooling tube and putting its cap into the cap detection well of the device; putting the pooling tube into the device; picking up and opening a sample tube; putting the sample tube and its cap into the device; scanning the barcode of the sample tube; picking up a corresponding transfer pipette; peeling off the plastic bag package of the pipette; aspirating the liquid sample from the sample tube; dispensing the liquid sample into the pooling tube; confirming the pooling result; disposing of the used pipette; closing the pooled sample tube; closing the pooling tube; removing the pooling tube from the device; storing the pooling tube for molecular testing; removing the tube tray with sample tubes from the device; storing the tubes for potential confirmatory testing; removing the cap tray from the device; disposing of the cap tray into the biohazard waste bin; removing the pipette tray from the device; disposing of the pipette tray into the biohazard waste bin; removing the pooling cap tray from the device; disposing of the pooling cap tray into the biohazard waste bin; disinfecting the workplace; taking off and disposing of the gloves into the biohazard waste bin; showing the success mark. Of the steps described above, the following steps may be repeated until all sample tubes are pooled successfully: picking up and opening a sample tube; putting the sample tube and its cap into the device; scanning the barcode of the sample tube; picking up a corresponding transfer pipette; peeling off the plastic bag package of the pipette; aspirating the liquid sample from the sample tube; dispensing the liquid sample into the pooling tube; confirming the pooling result; disposing of the used pipette.

For a Dorfman sample pooling procedure using liquid samples or other strategies using liquid samples, the individual steps of internal algorithm may be categorized by three groups of steps during the device operation in the training mode: preparation for pooling, which may include instructional steps for a minimum level of biosafety (e.g., putting on gloves and disinfection) and setting up pooling materials (e.g., tubes, transfer pipettes, disposable trays); pooling, which may include instructional steps for the transfer of samples from individual sample collection tubes to a single tube for pooling; and medical waste disposal and disinfection, which may include instructional steps for the disposal of medical wastes (e.g., used pipettes, their individual packages, and disposable trays) and a minimum level of biosafety (e.g., disinfection and taking off gloves).

The interactive devices in the training mode may provide a certificate to a user when the user meets the requirements for the certification, the interactive devices disclosed herein may show a barcode that contains a link for an online website to register the user's information and provide a certificate to the user. The interactive devices may enable the user to post the certificate on professional social media to have chances to get high-quality jobs.

For a Dorfman sample pooling procedure as well as other pooling strategies, interactive medical devices may operate in an expert mode with internal algorithms that consist of several individual steps, including but not limited to: set-up of disposable protective elements, set-up of individual sample-holding elements in the compartment detecting sample-holding elements, identification of the individual sample-holding elements, set-up of lab/medical supplies in the compartment detecting the supplies, set-up of sample-transfer elements in the compartment detecting the sample-transfer elements, identification of the pooled sample-holding element, set-up of the pooled sample-holding element in the compartment detecting the pooled sample-holding element, opening of the pooled sample-holding element, opening of an individual sample-holding element, pick-up of a sample-transfer element, pooling of sample into the pooled sample-holding element, confirmation of the pooling performance, closing of the opened individual sample-holding element, closing of the opened pooled sample-holding element, removal of the pooled sample-holding element from the device, removal of the individual sample-holding elements and their protective element from the device, removal of disposable protective elements from the device, disposal of the protective elements, disinfection, removal of PPE from the user, and disposal of the used PPE.

For Dorfman sample pooling procedure using liquid samples or other strategies using liquid samples, interactive medical devices may operate in an expert mode with internal algorithms that consist of several individual steps, including but not limited to setting up a tube tray with sample tubes; scanning barcodes of tubes; setting up disposable trays (e.g., a pipette tray, a cap tray, and a pooling cap tray); scanning the barcode of the pooling tube; opening the pooling tube and putting its cap into the cap detection well of the device; opening the sample tube and putting the tube and its cap into the device; picking up a corresponding transfer pipette; pooling the liquid sample from the sample tube into the pooling tube; confirming the pooling result; disposing of the used pipette; closing the pooled sample tube; closing and removing the pooling tube from the device; removing the tube tray with sample tubes from the device; removing all disposable trays (e.g., a pipette tray, a cap tray, and a pooling cap tray) from the device and disposing of them into the biohazard waste bin; disinfecting the workplace; taking off and disposing of the gloves into the biohazard waste bin; and showing the success mark. Of the steps described above, the following steps may be repeated until all sample tubes are pooled successfully: opening the sample tube and putting the tube and its cap into the device; picking up a corresponding transfer pipette; pooling the liquid sample from the sample tube into the pooling tube; confirming the pooling result; disposing of the used pipette; and closing the pooled sample tube.

For a Dorfman sample pooling procedure using liquid samples or other sample pooling procedures, the individual steps of internal algorithm may be categorized by three groups of steps during the device operation in the expert mode: preparation for pooling, which may include fewer quick steps for disinfection and materials (e.g., tubes, transfer pipettes, disposable trays) setup; pooling, which may include a single step for the transfer of sample from individual sample collection tubes to a single tube for pooling; and, medical waste disposal and disinfection, which may include fewer quick steps for the disposal of medical wastes (e.g., used pipettes, their individual packages, and disposable trays) and a minimum level of biosafety (e.g., disinfection and taking off gloves).

The interactive medical devices in the expert mode may record the performance of pooling for every cycle on the internal and external digital repository. The recorded data saved on the digital repository may be traceable, helping a user to check whether the sample pooling has been performed correctly for specific clinical samples.

Interactive devices may provide an entertainment mode to a user in order to engage the user for the sample handling procedures. Interactive devices in the entertainment mode may give a score or point when the user succeeds in each step of sample handling procedure. For example, the user may get scores when the user passes biosafety-related steps such as wearing gloves step, disinfection step. The user may also get scores or points when the user performs the aspiration and dispense steps successfully. The devices may save the scores on internal and external digital storage. The interactive devices may display a barcode to let the user access a mobile software environment for social networks or entertainment platforms where users can share their records with each other on a global scale. For sample pooling procedures, the social network platform may provide the user's records including, but not limited to: an acquired score in the previous single sample handling cycle, an average score for all single sample handling cycles, a total acquired score during a week, a total acquired score, certification, institute, a total number of performed sample handling process, a total number of errors that the user has made, an average number of errors per a single sample handling process that the user has made, parameters of sample handling protocols: pool size, the target volume, etc. The entertainment systems including device platform and mobile platform may provide a shared score or information and engage users through some enjoyable challenges or missions for user engagement. The same sequence of the internal algorithms in the training mode can be used for entertainment mode.

Interactive medical devices may provide an individual sample tracking system that helps a user locate individual samples that were pooled into a single pooled sample-handling element when the pooled sample-handling element registers the pool as positive for a target of interest. The user may be able to identify a container, for example, by scanning its barcode, as a storage element for individual sample-holding elements after finishing the sample pooling procedures. When the device identifies the container, the device may save the information on the internal and external digital repositories. From the records of the unique identifiers of the individual sample-holding elements, and/or the protective element for the individual sample-holding elements, and/or the container, the devices may create a record of the sample pool, thereby allowing downstream results to be linked to individual samples and faster accessioning of individual sample-holding elements if re-testing is needed. When the devices identify a single pooled sample-holding element before starting the sample pooling procedures, the devices may show the coordinates of individual sample-holding elements and their storage container associated with the single pooled sample-holding element.

Interactive devices may provide a sample management system that helps a user locate and/or access and/or manage samples in a storage unit. The management system may check whether the user accesses a correct storage where at least one sample of interest or sample-holding element has been stored and give the user feedback using at least one perceptible indication. The management system may check whether or not the user removes the correct sample of interest from the storage unit and give the user feedback using at least one perceptible indication. The management system may provide the user with information on the sample of interest, including, but not limited to, its physical location, the patient's identification, the sample type, the date and time of sample collection, the remaining volume (and/or weight) of the sample, and/or its testing/access history. The management system may access internal, external, and/or virtual digital repositories, linked to an EMR system and/or LIMS, and may read/write information on the repositories. When the interactive devices receive information on the sample, sample-holding elements, and/or the user's behaviors, the interactive devices may guide the user for related handling procedures through perceptible indications.

As described above, interactive medical devices for sample pooling may integrate with an EMR system or systems. The interactive medical devices for sample pooling may additionally or alternatively be integrated with a LIMS system or systems. The interactive medical devices disclosed herein may be able to connect to the LIMS and/or EMR using wired and/or wireless connection technology for data transfer such as cellular network, Ethernet connection, and Bluetooth. The interactive medical devices may have security technology to protect data from outside network and connect with other systems including LIMS and EMR system.

Interactive devices may record a user's performance of sample handling procedures. The information may include but not limited to the elapsed time for each step to be performed, the handling performance (e.g., average pooled volume from each sample), and the error rates for each step. The interactive devices may provide adaptive feedback functions that change the timing of cues (e.g., perceptible indicators) based on the pace and accuracy with which the user is performing individual steps. The interactive devices may provide customized operation for sample handling procedures. Some components, compartments, or functions of the interactive devices may be activated or inactivated by users who want to customize the devices, increasing usability based on the users' preference for the users' sample handling workflow. The interactive devices may provide customized algorithms to a user based on recorded data by adaptive feedback to maximize the efficiency of sample handling workflow. The interactive devices may be able to predict the user's performance of sample handling based on cumulated data by adaptive feedback.

A number of embodiments of the disclosure have been described. Nevertheless, it will be understood that various modifications may be made without departing from the spirit and scope of the present disclosure. Accordingly, other embodiments are within the scope of the following claims.

1. An instructional medical device comprising a modular assembly, wherein the modules of the modular assembly comprise:
one or more sample-transfer modules, wherein each sample-transfer module is configured to hold one sample-transfer element tray and one or more sample-transfer elements;
a scanning module configured to read an identifier from an item;
a processing module configured to hold one or more post-processing sample containers, one post-processing sample container closure tray, and one or more post-processing sample container closures;
one or more sample-container modules, wherein each sample-container module is configured to hold one sample container tray and one or more sample containers;
one or more container-closure modules, wherein each container-closure module is configured to hold one sample container closure tray and one or more sample container closures;
one or more multi-scanning modules, wherein each multi-scanning module is configured to read one or more identifiers from the sample container tray and the one or more sample containers;
one or more waste container modules, wherein each waste container module is configured to contain one or more objects disposed of during use of the instructional medical device; and
a display module configured to display information related to the instructional medical device,
and wherein the instructional medical device further comprises an electronics system communicating to components within the modules of the modular assembly.

2. The instructional medical device according to claim 1, wherein the instructional device is configured for sample pooling.

3.-4. (canceled)

5. The instructional medical device according to claim 1, wherein the processing module comprises:
one or more post-processing sample container holders;
one or more post-processing sample container weight load cells, wherein each post-processing sample container weight load cell measures weight of each post-processing sample container held within a corresponding one of the one or more post-processing sample container holders;
one or more post-processing sample container status indicators, wherein each post-processing sample container status indicator displays status based on a weight measured by a corresponding one of the one or more post-processing sample container weight load cells;
one or more post-processing sample container closure detection wells;
one or more post-processing sample container closure sensors, wherein each post-processing sample container closure sensor detects the presence of an individual post-processing sample container closure located within a corresponding one of the one or more post-processing sample container closure detection wells;
a post-processing sample container closure tray sensor; and
a post-processing sample container closure tray sensor indicator for detecting the presence of a post-processing sample container closure tray loaded onto the processing module.

6.-11. (canceled)

12. The instructional medical device according to claim 1, wherein the instructional medical device has at least one of the following operational modes:
a training mode for helping a user learn about the operation of the instructional medical device, helping a user improve sample handling skills, or training a user of handling skill acquisition from the instructional medical device or skill adaptation to the handling process without assistance from the device; and
an expert mode for use by a trained user.

13. The instructional medical device according to claim 1, wherein at least one of the one or more sample-transfer modules comprises a pipette module; the sample-transfer element tray comprises a pipette tray; at least one of the one or more sample-transfer elements comprises a pipette; the scanning module comprises a single barcode module; the identifier from an item comprises a barcode from the item; the processing module comprises a pooling module; at least one of the one or more post-processing sample containers comprises a pooling tube; the post-processing sample container closure tray comprises a pooling tube cap tray; at least one of the one or more post-processing sample container closures comprises a pooling tube cap; at least one of the one or more sample-container modules comprises a tube module; the sample container tray comprises a sample tube tray; at least one of the one or more sample containers comprises a sample tube; at least one of the one or more container-closure modules comprises a cap module; the sample container closure tray comprises a sample tube cap tray; at least one of the one or more sample container closures comprises a sample tube cap; at least one of the one or more multi-scanning modules comprises a multi-barcode module; and at least one of the one or more waste container modules comprises a waste bin module.

14. The instructional medical device according to claim 1, wherein the modules are oriented to provide a left-to-right workflow and wherein the one or more sample-transfer modules are located at a far left side of the modular assembly and the one or more multi-scanning modules are located at a far right side of the modular assembly, and wherein the scanning module is located adjacent to the sample-transfer module and the one or more container-closure modules are located adjacent to the one or more multi-scanning modules and the processing module are located adjacent to the scanning module and the one or more sample-container modules are located adjacent to the one or more container-closure modules and the processing module.

15. A method for sample handling using a sample handling device having object sensing elements, the method comprising:
detecting disposal of objects disposed of during the sample handling process, including one or more wipes or towels used for cleaning, one or more pieces of peeled-off plastic bag package of an individual disposable sample-transfer element, one or more used sample-transfer elements after the dispensation of sample, one or more sample container trays used during the process, one or more sample container closure trays used during the process, one or more sample-transfer element trays used during the process, one or more post-processing sample container closure trays used during the process, and one or more gloves;
providing status to a user regarding the presence of the objects disposed of during the process, with at least one perceptible indication element of the sample handling device;
providing status to a user regarding the movement of the disposal container due to the disposal action during the process, with at least one perceptible indication element of the sample handling device;
detecting the user's action of disposal done by the user's hand, food, or body;
providing status to a user regarding the user's action of disposal during the process, with at least one perceptible indication element of the sample handling device;
detecting presence of a sample container tray holding one or more sample containers, a sample-transfer element tray holding one or more sample-transfer elements, a sample container closure tray having one or more sample container closure detection wells, and a post-processing sample container closure tray in the sample handling device with at least one object sensing element;
providing status to a user regarding the presence of the sample container tray, the sample container closure tray, and the post-processing sample container closure tray with at least one perceptible indication element of the sample handling device;
detecting presence of a post-processing sample containers in a post-processing sample container holder of the sample handling device, and the post-processing sample container closure in the post-processing sample container closure tray in the sample handling device with at least one object sensing element;
providing status to the user regarding the presence of the one or more post-processing sample containers and the post-processing sample container closure tray with a least one perceptible indication element of the sample handling device;
detecting presence of a sample container closure in a sample container closure detection well with at least one object sensing element;
providing status to the user regarding the presence of the sample container closure with at least one perceptible indication element of the sample handling device;
detecting removal of a sample-transfer element from the sample-transfer element tray with at least one object sensing element;
providing status to the user regarding the removal of the sample-transfer element with at least one perceptible indication element of the sample handling device;
detecting dispensation of material from a sample container into the post-processing sample container using the sample-transfer element with at least one object sensing element;
providing status to the user regarding the dispensation of material into the post-processing sample container with at least one perceptible indication element of the sample handling device;
detecting removal of the post-processing sample container closure from the post-processing sample container closure tray with at least one object sensing element;
providing status to the user regarding the removal of the post-processing sample container closure with at least one perceptible indication element of the sample handling device;
detecting removal of the post-processing sample container from the post-processing sample container holder with at least one object sensing element; and,
providing status to the user regarding the removal of the post-processing sample container with at least one perceptible indication element of the sample handling device.

16. The method according to claim 15, wherein the following steps are repeated until all sample containers in the one or more sample container trays are handled:
detecting disposal of objects disposed of during the sample handling process, including one or more wipes or towels used for cleaning, one or more pieces of peeled-off plastic bag package of an individual disposable sample-transfer element, one or more used sample-transfer elements after the dispensation of sample, one or more sample container trays used during the process, one or more sample container closure trays used during the process, one or more sample-transfer element trays used during the process, one or more post-processing sample container closure trays used during the process, and one or more gloves;
providing status to a user regarding the presence of the objects disposed of during the process, with at least one perceptible indication element of the sample handling device;
providing status to a user regarding the movement of the disposal container due to the disposal action during the process, with at least one perceptible indication element of the sample handling device;
detecting the user's action of disposal done by the user's hand, food, or body;
providing status to a user regarding the user's action of disposal during the process, with at least one perceptible indication element of the sample handling device;
detecting presence of a sample container closure in a sample container closure detection well;
providing status to the user regarding the presence of the sample container closure with at least one least one perceptible indication element of the sample handling device;
detecting removal of a sample-transfer element from the sample-transfer element tray with at least one sensor contained within the sample handling device;
providing status to the user regarding the removal of the sample-transfer element with at least one perceptible indication element of the sample handling device;
detecting dispensation of material from a sample container into the post-processing sample container using the sample-transfer element with a post-processing sample container sensor element; and
providing status to the user regarding the dispensation of material into the post-processing sample container with at least one perceptible indication element of the sample handling device.

17.-24. (canceled)

25. The method according to claim 15, wherein at least one sample-transfer element comprises at least one or more sample-transfer elements selected from at least one of the following elements: pipettes, aspirators, dispensers, droppers, samplers, collectors, inoculating tools, cottons, swabs, syringes, forceps, tweezers, scalpels, needles, scoops, spatulas dippers, sponges, cups, papers, scrapers, and toothpicks.

26.-43. (canceled)

44. A modular system for handling samples, wherein the system comprises one or modules, and wherein the modular system comprises: one or more object detection sensors, wherein each object detection sensors comprises: an infrared emitter directed towards an object in a first direction and an infrared receiver directed towards an object in a second direction, wherein the first direction is offset from second direction and wherein the infrared receiver receives maximum infrared light when an object is present and minimum infrared light when the object is absent; one or more motion detection sensors, wherein each motion detection sensor comprises at least one passive infrared sensor that detects differential changes of infrared light due to a user's action in a detecting area; and one or more weighing scales, wherein each weighing scale detects the weight of an object, including a post-processing sample container or a material disposed of during a sample handling process.

45. The modular system according to claim 44, wherein a first module of the modular system comprises a processing module configured to hold one or more post-processing sample containers, one or more post-processing sample container closure trays, and one or more post-processing sample container closures in a post-processing sample container closure tray, wherein the processing module comprises:
one or more post-processing sample container holders configured to hold the one or more post-processing sample containers;
one or more post-processing sample container weight load cells, wherein each post-processing sample container weight load cell measures weight of a corresponding one of the one or more post-processing sample containers held within the post-processing sample container holders;
one or more post-processing sample container status indicators, wherein each post-processing container status indicator displays status based on a weight measured by a corresponding one of the one or more post-processing sample container weight load cells;
one or more post-processing sample container closure detection wells configured to receive the post-processing sample container closure tray;
one or more post-processing sample container closure sensors comprising at least one of the object detection sensors, wherein each post-processing sample container closure sensor detects the presence of an individual post-processing sample container closure located within a corresponding one of the one or more post-processing sample container closure detection wells;
a post-processing sample container closure tray sensor indicator configured for detecting the presence of the post-processing sample container closure tray when loaded onto the processing module, wherein the post-processing sample container closure sensor comprises an infrared receiver directed towards a location of the post-processing sample container closure tray located in the post-processing sample container closure detection well; and
a post-processing sample container closure tray sensor indicator configured for displaying status related to the post-processing sample container closure and the post-processing sample container closure tray.

46. The modular system according to claim 45, further comprising a second module of the modular system, wherein the second module comprises one or more sample-container modules configured to hold one or more sample container trays and one or more sample container in a sample container tray; wherein each of the one or more sample-container module comprises:
one or more sample container receiving slots;
one or more sample container receiving slot status indicators, wherein each sample container receiving slot status indicator corresponds to one of the one or more sample container receiving slots;
one or more sample container sensors comprising one or more of the object detection sensors, wherein each sample container sensor detects the presence of an individual sample container within a corresponding one of the one or more sample container receiving slots; and
a sample container tray sensor for detecting the presence of a sample container tray loaded onto the sample-container module, wherein the sample container tray sensor comprises an infrared receiver directed towards a location of the sample container tray when located in the sample-container module.

47. The modular system according to claim 46, further comprising a third module of the modular system, wherein the third module comprises one or more container-closure modules configured to hold one or more sample container closure trays and one or more sample container closures in a sample container closure tray;
wherein each of the one or more container-closure modules comprises:
one or more sample container closure detection wells;
one or more sample container closure detection well status indicators, wherein each sample container closure detection well status indicator corresponds to one of the one or more sample container closure detection wells;
one or more sample container closure detection sensors comprising one or more object detection sensors, wherein each sample container closure detection sensor detects the presence of an individual sample container closure within a corresponding one of the one or more sample container closure detection wells; and
a sample container closure tray sensor detecting the presence of a sample container closure tray loaded onto the container-closure module, wherein the sample container closure tray sensor comprises an infrared receiver directed towards a location of the sample container closure tray when located in the container-closure module.

48. The modular system according to claim 47, further comprising a fourth module of the modular system, wherein the fourth module comprises one or more multi-scanning modules configured to read barcodes from the sample container tray and the one or more sample containers; wherein each of the one or more multi-scanning modules comprises:
one or more sample container barcode scanners, wherein each one of the one or more sample container scanners is oriented to read one or more barcodes from the sample container held within a corresponding sample container receiving slot of the sample-container module; and
a sample container tray barcode scanner, wherein the sample container tray scanner is oriented to read one or more barcodes from the sample container tray when located in the sample-container module.

49. The modular system according to claim 48, further comprising a fifth module of the modular system, wherein the fifth module comprises one or more sample-transfer modules configured to hold one or more sample-transfer element trays and one or more sample-transfer elements in a sample-transfer element tray;
wherein each of one or more sample-transfer modules comprises:
one or more sample-transfer element receiving slots;
one or more sample-transfer element-sensing sensors comprising one or more object detection sensors, wherein each sample-transfer element sensor detects presence of an individual sample-transfer element within a corresponding one of the one or more sample-transfer element slots;
one or more sample-transfer element receiving slot status indicators, wherein each sample-transfer element receiving slot status indicator corresponds to one of the one or more sample-transfer element receiving slots and wherein each sample transfer element receiving slot status indicator displays status according to presence detected by the sample-transfer element sensing sensor of the corresponding one of the one or more sample-transfer element slots;
a sample-transfer element tray sensor detecting the presence of a sample-transfer element tray loaded onto the sample-transfer module, wherein the sample-transfer element tray sensor comprises an infrared receiver directed towards a location of the sample-transfer element tray when located in the sample-transfer module.

50. The modular system according to claim 49, further comprising a sixth module of the modular system, wherein the sixth module comprises a scanning module configured to read at least one barcode from an item; wherein the scanning module comprises:
a barcode scanner oriented to detect one or more barcodes of the item held external to the scanning module; and
a scanning status indicator for displaying status based on scans performed by the barcode scanner.

51. The modular system according to claim 50, further comprising a seventh module of the modular system, wherein the seventh module comprises a display module, the display module in electrical communication with the processing, sample-container, container-closure, multi-scanning, sample-transfer, and scanning modules and the display module comprising a display configured for displaying language-agnostic graphics.

52. The modular system according to claim 51, further comprising an eighth module of the modular system, wherein the eighth module comprises one or more waste container modules configured to hold one or more objects disposed of during the sample handling process; wherein each of the one or more waste container modules comprises:
one or more disposal containers where any objects (or wastes) are disposed of;
one or more set of disposal container status indicators, wherein each set of disposal container status indicators corresponds to one of disposal containers;
one or more disposal motion detection sensors, wherein each disposal motion detection sensor detects the user's disposal action by the user's hand, foot, or body;
one or more disposal container weighing load cells scales, wherein each disposal container weight load cell measures weight or weight changes of each disposal container; and
one or more waste detection sensors, wherein each waste detection sensor detects one or more objects passing through the detection area.

53. The modular system according to claim 44, wherein the object detection sensors provide a detection within one second or less.

54. The modular system according to claim 44, wherein the samples for pooling comprise samples from the group consisting of: cells, tissues, proteins, bacteria, fungi, archaea, protists, germs, parasites, viruses, nucleic acids, bacteriophages, vesicles, lipids, water, oil, blood, plasma, serum, saliva, urine, sputum, semen, sperm, synovial fluids, mucus, cerebrospinal fluids, peritoneal fluids, ascites, sweat, vaginal fluids, amniotic fluids, tears, blisters, cyst fluids, feces, wax, hair, and inorganic ions.

55.-64. (canceled)
